(19) United States
(12) Patent Application Publication
(10) Pub. No.: US 2023/0151401 A1
(43) Pub. Date: May 18, 2023

(54) METHOD OF REDUCING AND RECYCLING OXIDIZED FLAVIN COFACTORS

(71) Applicant: Oxford University Innovation Limited, Oxford (GB)

(72) Inventors: Kylie VINCENT, Oxford (Oxfordshire) (GB); Sarah CLEARY, Begbroke (Oxfordshire) (GB); Shiny Joseph SRINIVASAN, Oxford (Oxfordshire) (GB); Holly REEVE, Begbroke (Oxfordshire) (GB)

(21) Appl. No.: 17/920,561

(22) PCT Filed: Apr. 23, 2021
(86) PCT No.: PCT/GB2021/051000

§ 371 Date: Oct. 21, 2022

(30) Foreign Application Priority Data
Apr. 24, 2020 (GB) 2006047.1

(51) Int. Cl. C12P 19/30

(52) U.S. Cl. CPC C12P 19/30; C12Y 101/98; C12Y 103/01; C12Y 105/01; C12Y 111/01; C12Y 111/02; C12Y 112/99006; C12Y 114/14

(57) ABSTRACT

The invention relates to an enzymatic method for producing a reaction product. A method of recycling a biological cofactor is also provided. The invention also relates to systems and apparatuses for conducting such methods.

The project leading to this application has received funding from the European Research Council (ERC) under the European Union's Horizon 2020 research and innovation programme (grant agreement No 819580).

### FIELD OF THE INVENTION

The present invention relates to methods for producing a reaction product from a reactant. The invention also relates to methods of reducing oxidised flavin cofactors and to methods of recycling such flavin cofactors. The invention further relates to systems and apparatuses for the production of such reaction products and the recycling of such cofactors.

### BACKGROUND

Chemical manufacturing processes are typically associated with many environmental concerns. The reagents such as catalysts used are often non-renewable and/or toxic. Extreme operating conditions are typically required, such as elevated temperatures and pressures, with the provision of such conditions being energy inefficient. Toxic solvents are often needed in order to achieve satisfactory yields. Furthermore, the reagents used are often non-selective requiring complex synthetic strategies in order to selectively process only desired functional groups within molecules.

Biological catalysis is an approach that has been suggested to address these and related issues. This approach exploits the exquisite chemical control offered by biological systems such as enzymes to process their chemical substrates. Enzymatic processing of chemical reagents offers advantages compared to traditional chemical processing methods. Enzymes are renewable and biodegradable, and thus overcome environmental issues regarding the production and disposal of chemical catalysts. Enzymes are typically nonhazardous and nontoxic, thus addressing safety concerns associated with chemical catalysts. Enzymes typically operate under moderate temperatures and at atmospheric pressure, thus reducing the energy demands associated with conventional chemical processing. Enzymes are also typically highly selective as regards their chemical substrate, and approaches such as rational enzyme engineering and directed mutagenesis continue to expand the range of reactions that can be undertaken. Enzymatic catalysis thus provides many advantages compared to conventional chemical approaches.

Whilst enzymatic catalysis has great potential, its use in industry has been limited. One key difficulty has been in the provision of robust systems for recycling enzyme cofactors.

Cofactors are non-protein chemical compounds that play an essential role in many enzyme catalysed biochemical reactions, and which typically act to transfer chemical groups between enzymes. Cofactors are also sometimes known as “co-substrates” reflecting their processing by an enzyme in the course of its catalysing of its primary reaction. By way of illustration, a redox enzyme which catalyses an oxidation reaction of a reagent to produce a product may couple that oxidation to the reduction of a cofactor as an electron sink. In this case, the overall reaction catalysed by the enzyme may be represented as:

reduced reagent+oxidised cofactor→oxidised product+reduced cofactor

Similarly, enzymes which catalyse the reduction of a reagent to produce a product typically couple that reduction with the oxidation of a cofactor as a source of electrons or reducing equivalents such as hydride ions:

oxidised reagent+reduced cofactor→reduced product+oxidised cofactor

Biological use of cofactors is not limited to simple redox reactions as represented above but is also involved in more complex reactions such as atom insertion reactions, rearrangement reactions, etc.

There are many cofactors known, including those that occur in nature and synthetic compounds which are designed to have specific properties such as precisely tuned redox potentials, improved stability, etc. One key natural cofactor is nicotinamide adenine dinucleotide (NAD). In vivo, reduction of the oxidised cofactor (NAD⁺) by hydride transfer from a reductant yields the reduced cofactor (NADH). The reduced cofactor can be coupled to enzymatic reduction of an oxidised centre (typically an oxidised carbon centre) to yield a reduced centre, in accordance with the general schemes shown above.

A difficulty which has limited the industrial exploitation of enzymes which rely on cofactors to catalyse relevant reactions is providing sufficient cofactor for the enzyme to use. One option is to provide the cofactor in superstoichiometric quantities relative to the reagent at issue. However, the high cost and typically low stability of reduced cofactor molecules means that this is not a viable approach. It is thus necessary that systems for regenerating cofactor molecules in their desired form (i.e., recycling the cofactor molecule) are used.

Current industrial practices for enzymatic NAD(P)H recycling rely on a superstoichiometric quantity of a carbon-based sacrificial reductant. For example, NAD(P)H is a cofactor for many enzymes used in reduction reactions. The reduction of the reagent to produce the desired product is linked to the enzymatic oxidation of NAD(P)H to NAD(P)+. To regenerate NAD(P)H for subsequent enzyme cycles, glucose or isopropanol is typically used as a sacrificial reductant. However, this leads to additional cost, generates waste products, and requires additional downstream processing steps of the desired product. It is also atom inefficient. In view of these difficulties, there have been extensive efforts in recent years to provide improved methods for recycling NAD(P)H, and some recent developments are promising.

A second important class of cofactor in vivo are the flavins. A variety of flavin cofactors exist, including flavin mononucleotide (FMN), flavin adenine dinucleotide (FAD), and riboflavin. Many enzymes which couple industrially useful reactions to flavin processing exist. However, unlike the situation for NAD(P)+/NAD(P)H dependent enzymes, widespread industrial exploitation of such flavin-utilising enzymes has been prevented by a lack of suitable means for recycling the flavin cofactor.

Some attempts to provide flavin recycling systems have proposed electrochemical reduction of oxidised flavin cofactors. However, in practice such systems are associated with many technical drawbacks. Electrochemical systems are typically difficult to incorporate into industrially relevant contexts. The electrodes used typically require costly materials such as precious metals and highly-processed carbon materials, the production of which is associated with environmental issues and is energy inefficient. Electrochemical side-reaction of the reagents or products may limit the overall efficiency of the reaction process. Furthermore, electrodes are typically subject to fouling by reaction by-products, the reagents or products themselves, or other impurities that may be present.

Accordingly, there is a pressing need for improved methods of recycling flavin cofactors. In particular, there is a need for methods for reducing an oxidised flavin cofactor such that the reduced cofactor thus obtained can be used in downstream enzyme-catalysed reactions. There is specifically a need for methods that avoid the requirement for expensive or dangerously reactive chemical reagents; that are atom efficient; that avoid difficulties associated with electrochemical processing of reagents; that do not rely on the use of expensive sacrificial reductants; and/or that avoid the generation of by-products. The present invention aims to address some or all of these problems.

### SUMMARY OF THE INVENTION

The inventors have surprisingly found that it is possible to use hydrogen as a reductant in order to reduce an oxidised flavin cofactor. The hydrogen is processed by a hydrogen-cycling enzyme such as a hydrogenase. Surprisingly, the inventors have found that hydrogenases which do not interact with flavin cofactors in vivo can still enzymatically reduce such cofactors using the electrons generated by hydrogen oxidation. The process is environmentally clean as the hydrogenase enzymes used are renewable and biodegradable. Unlike conventional hydrogen oxidation catalysts such as precious metals, the reactions catalysed by hydrogenases are highly specific and do not lead to unwanted side-reaction. Hydrogenases operate under readily accessible conditions and are amenable to exploitation in industrial contexts such as known reactors (including, but not limited to, hydrogenation reactors). They are not susceptible to fouling by reagent, product or cofactor molecules. By utilising hydrogen as the reductant, the reaction is atom efficient.

Accordingly, the invention provides a method of producing a reaction product, comprising:
i) contacting an oxidised flavin cofactor and molecular hydrogen (¹H₂) or an isotope thereof with a first polypeptide which is a hydrogenase enzyme or a functional fragment or derivative thereof under conditions such that the oxidised flavin cofactor is reduced to form a reduced flavin cofactor; and
ii) contacting the reduced flavin cofactor and a reactant with a second polypeptide which is an oxidoreductase or a functional fragment or derivative thereof under conditions such that (a) the oxidised flavin cofactor is regenerated; and (b) the second polypeptide catalyses the formation of the reaction product from the reactant.

Preferably, the method comprises:
i) contacting an oxidised flavin cofactor and molecular hydrogen (¹H₂) or an isotope thereof with a first polypeptide which is a hydrogenase enzyme or a functional fragment or derivative thereof under conditions such that the first polypeptide oxidises the hydrogen to produce protons and electrons, and transfers the electrons to the oxidised flavin cofactor, thereby reducing the oxidised flavin cofactor to form a reduced flavin cofactor; and
ii) contacting the reduced flavin cofactor and a reactant with a second polypeptide which is an oxidoreductase or a functional fragment or derivative thereof under conditions such that (a) electrons are transferred from the reduced flavin cofactor to an electron acceptor and/or hydride ions are transferred from the reduced flavin cofactor to a hydride ion acceptor; (b) the oxidised flavin cofactor is regenerated; and (c) the second polypeptide catalyses the formation of the reaction product from the reactant.

Preferably, the provided method is repeated multiple times thereby recycling the cofactor.

This method is illustrated schematically in FIG. 3.

Typically, the oxidised cofactor is selected from flavin mononucleotide (FMN), flavin adenine dinucleotide (FAD), riboflavin, or a derivative thereof. Preferably, the oxidised cofactor is flavin mononucleotide (FMN) or a derivative thereof or flavin adenine dinucleotide (FAD) or a derivative thereof.

Usually, the first polypeptide transfers the electrons to the oxidised flavin cofactor via an intramolecular electronically-conducting pathway. The intramolecular electronically-conducting pathway often comprises a series of [FeS] clusters. Preferably, reduction of the oxidised flavin cofactor takes place at an [FeS] cluster within the first polypeptide.

Preferably, the first polypeptide does not comprise a native flavin active site for NAD(P)⁺ reduction.

Preferably, the first polypeptide is an uptake hydrogenase or a hydrogen-sensing hydrogenase. Preferably, the first polypeptide is a hydrogenase of class 1 or 2b. References to hydrogenase classes such as class 1 and class 2b refer to the established Vignais classification scheme described by Vignais and Billoud, Chem. Rev. 2007, 107, 4206-4272, which is known to those skilled in the art.

Preferably, the first polypeptide is selected from or comprises:
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Ralstonia eutropha regulatory hydrogenase moiety (SEQ ID NOs: 8 and/or 9) or an amino acid sequence having at least 60% homology therewith;
v) the amino acid sequence of Aquifex aeolicus hydrogenase 1 (SEQ ID NO:10 and/or 11) or an amino acid sequence having at least 60% homology therewith;
vi) the amino acid sequence of Hydrogenovibrio marinus hydrogenase (SEQ ID NOs: 12 and/or 13) or an amino acid sequence having at least 60% homology therewith;
vii) the amino acid sequence of Thiocapsa roseopersicina hydrogenase (SEQ ID NOs: 14 and 15) or an amino acid sequence having at least 60% homology therewith;
viii) the amino acid sequence of Alteromonas macleodii hydrogenase (SEQ ID NOs: 16 and/or 17) or an amino acid sequence having at least 60% homology therewith;
ix) the amino acid sequence of Allochromatium vinosum membrane bound hydrogenase (SEQ ID NOs: 18 and/or 19) or an amino acid sequence having at least 60% homology therewith;
x) the amino acid sequence of Salmonella enterica serovar Typhimurium LT2 nickel-iron hydrogenase 5 (SEQ ID NO: 20 and/or 21) or an amino acid sequence having at least 60% homology therewith;
xi) the amino acid sequence of Desulfovibrio vulgaris Miyazaki F hydrogenase (SEQ ID NO: 23 and/or 24) or an amino acid sequence having at least 60% homology therewith;
   or a functional fragment, derivative or variant thereof.

Preferably, in one embodiment, the second polypeptide comprises the electron acceptor and/or hydride ion acceptor. Typically, the second polypeptide comprises a prosthetic group for oxidising the reduced flavin cofactor. This method is illustrated schematically in FIG. 3.

Preferably, in another embodiment, the electron acceptor and/or hydride ion acceptor comprises a molecular substrate. Typically, the molecular substrate comprises O₂. This method is illustrated schematically in FIG. 4.

Preferably, the second polypeptide is a flavin-accepting oxidoreductase, or a functional fragment, derivative or variant thereof. Preferably, the second polypeptide is a flavin-dependent oxidoreductase, or a functional fragment, derivative or variant thereof. Typically, the second polypeptide is a monooxygenase, halogenase, nitro reductase, ene-reductase, peroxidase, or haloperoxidase, or a functional fragment, derivative or variant thereof. Often, the second enzyme is selected from Enzyme Commission (EC) classes 1.1.98.; 1.3.1.; 1.5.1.; 1.6.99.; 1.7.1.; 1.7.99.; 1.11.1.; 1.11.2.; 1.14.14.; and 1.14.99.; or a functional fragment, derivative or variant thereof.

In one embodiment, first polypeptide and/or the second polypeptide are preferably in solution. In another embodiment, the first polypeptide and/or the second polypeptide is immobilised on a solid support. The first polypeptide and the second polypeptide may be attached together, as illustrated schematically in FIG. 5. The first polypeptide and/or the second polypeptide may be comprised in a biological cell.

Preferably, the method is carried out under aerobic conditions. Typically, the method is carried out at a temperature of from about 20° C. to about 80° C.

Also provided is a method of reducing an oxidised flavin cofactor, comprising:
contacting the oxidised flavin cofactor and molecular hydrogen (¹H₂) or an isotope thereof with a first polypeptide which is a hydrogenase enzyme or a functional fragment or derivative thereof under conditions such that the oxidised flavin cofactor is reduced to form a reduced flavin cofactor;
wherein the first polypeptide does not comprise a native flavin active site for NAD(P)⁺ reduction.

This method is illustrated schematically in FIG. 1.

Preferably, said method further comprises the re-oxidation of the reduced flavin cofactor to regenerate the oxidised flavin cofactor. Typically, the reduction and reoxidation steps are repeated multiple times thereby recycling the cofactor.

This method is illustrated schematically in FIG. 2.

Preferably, in such methods, the oxidised flavin is as defined herein; the first polypeptide is as defined herein; the method is conducted under conditions as described herein; and/or the first polypeptide is immobilised on a solid support or is comprised in a biological cell.

The invention also provides a system for reducing an oxidised flavin cofactor, comprising:
a first polypeptide which is a hydrogenase enzyme or a functional fragment or derivative thereof,
the oxidised flavin cofactor; and
molecular hydrogen (¹H₂) or an isotope thereof;
   wherein the first polypeptide does not comprise a native flavin active site for NAD(P)⁺ reduction.

Also provided is a system for producing a reaction product, comprising:
a first polypeptide which is a hydrogenase enzyme or a functional fragment or derivative thereof;
a flavin cofactor;
a second polypeptide which is an oxidoreductase or a functional fragment or derivative thereof,
molecular hydrogen (¹H₂) or an isotope thereof, and
a reactant for conversion to said reaction product.

Preferably, in the systems provided herein, the flavin cofactor is as defined herein; the first polypeptide is as defined herein; and/or the second polypeptide if present is as defined herein.

Without being bound by theory, the inventors believe that, in the methods of the invention, electrons are typically abstracted from hydrogen by the first polypeptide and used to reduce the oxidised flavin cofactor. The reduction takes places at the first polypeptide. The product of the reduction is thus a reduced flavin cofactor. In embodiments of the invention in which the reduced flavin cofactor is exploited in the production of a reaction product from a reactant, the reduced flavin cofactor is typically oxidised at a second polypeptide. For example, the reduced flavin may be oxidised by the second polypeptide, e.g. at an active site or prosthetic group of the second polypeptide. The reduced flavin may be oxidised by an electron or hydride ion acceptor such as O₂. In these embodiments, the second polypeptide catalyses the formation of the product by reaction of the reagent with the oxidised flavin cofactor.

The second polypeptide thus catalyses the conversion of the reactant to the product. The reaction catalysed by the second polypeptide may for example be a reduction reaction, e.g. the reduction of a C═C double bond to a C—C single bond. Such reactions are catalysed by enzymes such as ene reductases. The reaction catalysed by the second polypeptide may be an atom insertion reaction such as the insertion of an oxygen atom into a chemical bond. Such reactions are catalysed by enzymes such as monooxygenases and peroxidases. The reaction may be a halogenation reaction. Such reactions are catalysed by enzymes such as halogenases and haloperoxidases. The reaction may be the reduction of a nitro group e.g. a nitroaromatic group, or a quinone; such reactions are catalysed by enzymes such as nitroreductases.

### DESCRIPTION OF THE SEQUENCE LISTING

SEQ ID NO: 1—the amino acid sequence of the Escherichia coli hydrogenase 1 (large subunit).
SEQ ID NO: 2—the amino acid sequence of the Escherichia coli hydrogenase 1 (small subunit).
SEQ ID NO: 3—the amino acid sequence of the Escherichia coli hydrogenase 2 (large subunit).
SEQ ID NO: 4—the amino acid sequence of the Escherichia coli hydrogenase 2 (small subunit).
SEQ ID NO: 5—the amino acid sequence of the Ralstonia eutropha membrane-bound hydrogenase moiety (HoxG).
SEQ ID NO: 6—the amino acid sequence of the Ralstonia eutropha membrane-bound hydrogenase moiety (HoxK).
SEQ ID NO: 7—the amino acid sequence of the Ralstonia eutropha membrane-bound hydrogenase moiety (HoxZ).
SEQ ID NO: 8—the amino acid sequence of the Ralstonia eutropha regulatory hydrogenase moiety (HoxB).
SEQ ID NO: 9—the amino acid sequence of the Ralstonia eutropha regulatory hydrogenase moiety (HoxC).
SEQ ID NO: 10—the amino acid sequence of the Aquifex aeolicus hydrogenase 1 (large subunit).
SEQ ID NO: 11—the amino acid sequence of the Aquifex aeolicus hydrogenase 1 (small subunit).
SEQ ID NO: 12—the amino acid sequence of the Hydrogenovibrio marinus hydrogenase (large subunit).
SEQ ID NO: 13—the amino acid sequence of the Hydrogenovibrio marinus hydrogenase (small subunit).
SEQ ID NO: 14—the amino acid sequence of the Thiocapsa roseopersicina hydrogenase HupL.
SEQ ID NO: 15—the amino acid sequence of the Thiocapsa roseopersicina hydrogenase HupS.
SEQ ID NO: 16—the amino acid sequence of the Alteromonas macleodii hydrogenase small subunit.
SEQ ID NO: 17—the amino acid sequence of the Alteromonas macleodii hydrogenase large subunit.
SEQ ID NO: 18—the amino acid sequence of the Allochromatium vinosum Membrane Bound Hydrogenase large subunit.
SEQ ID NO: 19—the amino acid sequence of the Allochromatium vinosum Membrane Bound Hydrogenase small subunit.
SEQ ID NO: 20—the amino acid sequence of the Salmonella enterica serovar Typhimurium LT2 nickel-iron hydrogenase 5 Large subunit.
SEQ ID NO: 21—the amino acid sequence of the Salmonella enterica serovar Typhimurium LT2 nickel-iron hydrogenase 5 Small subunit.
SEQ ID NO: 22—the amino acid sequence of the Escherichia coli cytochrome HyaC.
SEQ ID NO: 23—the amino acid sequence of the Desulfovibrio vulgaris Miyazaki F hydrogenase (large subunit).
SEQ ID NO: 24—the amino acid sequence of the Desulfovibrio vulgaris Miyazaki F hydrogenase (small subunit).
SEQ ID NO: 31—the amino acid sequence of Chromate Reductase, ‘TsOYE’ from Thermus scotoductus.
SEQ ID NO: 32—the amino acid sequence of NADPH Dehydrogenase 1, ‘OYE-1’, Saccharomyces pastorianus.
SEQ ID NO: 33: the amino acid sequence of NADPH Dehydrogenase 2, ‘OYE-2’, Saccharomyces cerevisiae strain ATCC 204508 S288c.
SEQ ID NO: 34: the amino acid sequence of NADPH Dehydrogenase, ‘YqjM’, Bacillus subtilis.
SEQ ID NO: 35: the amino acid sequence of Xenobiotic Reductase A, ‘XenA’, Pseudomonas putida.
SEQ ID NO: 36: the amino acid sequence of NADPH dehydrogenase, ‘FOYE-1’, ‘Ferrovum’ strain JA12.
SEQ ID NO: 37: the amino acid sequence of Oxidored_FMN domain-containing protein, ‘MgER’, Meyerozyma guilliermondii.
SEQ ID NO: 38: the amino acid sequence of Oxidored_FMN domain-containing protein, ‘CER’, Clavispora (Candida) lusitaniae.
SEQ ID NO: 39: the amino acid sequence of Tryptophan 2-Halogenase, ‘CmdE’, Chondromyces crocatus.
SEQ ID NO: 40: the amino acid sequence of Tryptophan 5-Halogenase, ‘PyrH’, Streptomyces rugosporus.
SEQ ID NO: 41: the amino acid sequence of Flavin-Dependent Tryptophan Halogenase, ‘RebH’, Lentzea aerocolonigenes (Lechevalieria aerocolonigenes) (Saccharothrix aerocolonigenes).
SEQ ID NO: 42: the amino acid sequence of Flavin-Dependent Tryptophan Halogenase, ‘PrnA’, Pseudomonas fluorescens.
SEQ ID NO: 43: the amino acid sequence of Thermophilic Tryptophan Halogenase, ‘Th-Hal’, Streptomyces violaceusnige.
SEQ ID NO: 44: the amino acid sequence of Tryptophan 6-Halogenase, ‘SttH’, Streptomyces toxytricini.
SEQ ID NO: 45: the amino acid sequence of KtzQ, ‘KtzQ’, Kutzneria sp. 744.
SEQ ID NO: 46: the amino acid sequence of Monodechloroaminopyrrolnitrin halogenase, ‘PrnC’, Pseudomonas fluorescens.
SEQ ID NO: 47: the amino acid sequence of FADH2-dependent halogenase, ‘PitA’, Pseudomonas protegens Pf-5.
SEQ ID NO: 48: the amino acid sequence of Halogenase, ‘PltM’, Pseudomonas fluorescens (strain ATCC BAA-477 NRRL B-23932 Pf-5).
SEQ ID NO: 49: the amino acid sequence of Flavin-Dependent Halogenase, ‘Clz5’, Streptomyces sp. CNH-287.
SEQ ID NO: 50: the amino acid sequence of Pyrrole Halogenase, ‘Bmp2’, Pseudoalteromonas piscicida.
SEQ ID NO: 51: the amino acid sequence of Non-Heme Halogenase, ‘Rdc2’, Metacordyceps chlamydosporia (Pochonia chlamydosporia).
SEQ ID NO: 52: the amino acid sequence of Tryptophan 6-Halogenase, ‘BorH’, uncultured bacteria.
SEQ ID NO: 53: the amino acid sequence of Styrene Monooxygenase, ‘StyA’, Pseudomonas sp.
SEQ ID NO: 54: the amino acid sequence of 4-Nitrophenol 2-Monooxygenase Oxygenase Component, ‘PheA1’, Rhodococcus erythropolis (Arthrobacter picolinophilus).
SEQ ID NO: 55: the amino acid sequence of 4-Hydroxyphenylacetate 3-Monooxygenase Oxygenase Component, ‘HpaB’, Klebsiella oxytoca.
SEQ ID NO: 56: the amino acid sequence of Chlorophenol Monooxygenase, ‘HadA’, Ralstonia pickettii (Burkholderia pickettii).
SEQ ID NO: 57: the amino acid sequence of Tetrachlorobenzoquinone Reductase, ‘PcpD’, Sphingobium chlorophenolicum.
SEQ ID NO: 58: the amino acid sequence of 2-Methyl-6-ethyl-4-monooxygenase Oxygenase Component, ‘MeaX’, Sphingobium baderi.
SEQ ID NO: 59: the amino acid sequence of Alkanesulfonate Monooxygenase, ‘SsuD’, Escherichia coli (strain K12).
SEQ ID NO: 60: the amino acid sequence of p-Hydroxyphenylacetate 3-Hydroxylase, Oxygenase Component, ‘C2-HpaH’, Acinetobacter baumannii (SEQ ID NO:60).
SEQ ID NO: 61: the amino acid sequence of FADH(2)-Dependent Monooxygenase, ‘TftD’, Burkholderia cepacia (Pseudomonas cepacia).
SEQ ID NO: 62: the amino acid sequence of 4-Nitrophenol 2-Monooxygenase, Oxygenase Component, ‘NphA1’, Rhodococcus sp.
SEQ ID NO: 63: the amino acid sequence of Putative dehydrogenase/oxygenase subunit, ‘VpStyA1’, Variovorax paradoxus (strain EPS).
SEQ ID NO: 64: the amino acid sequence of Oxygenase, ‘RoIndA1’ {from styA1 gene}, Rhodococcus opacus (Nocardia opaca).
SEQ ID NO: 65: the amino acid sequence of Smoa_sbd domain-containing protein, ‘AbIndA’, Acinetobacter baylyi (strain ATCC 33305 BD413 ADPI).
SEQ ID NO: 66: the amino acid sequence of 2,5-Diketocamphane 1,2-Monooxygenase 1, ‘CamP’, Pseudomonas putida (Arthrobacter siderocapsulatus).
SEQ ID NO: 67: the amino acid sequence of 3,6-Diketocamphane 1,6-Monooxygenase, ‘CamE36’, Pseudomonas putida (Arthrobacter siderocapsulatus).
SEQ ID NOs: 68 and 69: the amino acid sequence of Alkanal monooxygenase, alpha and beta chain, ‘LuxAB’, Vibrio harveyi (Beneckea harveyi); SEQ ID NOs: 70 and 71: the amino acid sequence of Alkanal monooxygenase, alpha and beta chain, ‘LuxAB’, Photorhabdus luminescens (Xenorhabdus luminescens).
SEQ ID NO: 72: the amino acid sequence of Alkane Monooxygenase, ‘LadA’, Geobacillus thermodenitrificans.
SEQ ID NO: 73: the amino acid sequence of EDTA Monooxygenase, ‘EmoA’, Chelativorans multitrophicus.
SEQ ID NO: 74: the amino acid sequence of Isobutylamine N-hydroxylase, ‘IBAH’, Streptomyces viridifaciens.
SEQ ID NO: 75: the amino acid sequence of ActVA 6 Protein, ‘ActVA-Orf6’, Streptomyces coelicolor.
SEQ ID NO: 76: the amino acid sequence of Pyrimidine Monooxygenase, ‘RutA’, Escherichia coli (strain K12).
SEQ ID NO: 77: the amino acid sequence of p-Hydroxyphenylacetate 2-Hydroxylase Reductase Component, ‘C1-HpaH’, Acinetobacter baumannii.
SEQ ID NO: 78: the amino acid sequence of FMN_red Domain-Containing Protein, ‘YdhA’, Bacillus subtilis subsp. natto (strain BEST195).
SEQ ID NO: 79: the amino acid sequence of NAD(P)H-Flavin Reductase, ‘Fre’, Escherichia coli (strain K12).
SEQ ID NO: 80: the amino acid sequence of 4-hydroxyphenylacetate 3-monooxygenase reductase component, ‘HpaC’, Escherichia coli.
SEQ ID NO: 81: the amino acid sequence of nitroreductase ‘NfsB’, Escherichia coli (strain K12).
SEQ ID NO: 82: the amino acid sequence of vanadium chloroperoxidase ‘CPO’ or ‘CiVHPO’, Curvularia inaequalis.
SEQ ID NO: 83: the amino acid sequence of aromatic unspecified peroxygenase ‘APO1’ or ‘AaeUPO’, Agrocybe aegerita (Blackpoplar mushroom) (Agaricus aegerita).

### BRIEF DESCRIPTION OF THE FIGURES

FIG. 1 shows a schematic diagram of the production of a reduced flavin cofactor from an oxidised flavin cofactor in accordance with the methods of the invention.

FIG. 2 shows a schematic diagram of the recycling of a flavin cofactor in accordance with the methods of the invention.

FIG. 3 shows a schematic diagram of the production of a product from a reactant in accordance with the methods of the invention, wherein electrons and/or hydride ions are transferred from the reduced flavin cofactor to an electron and/or hydride ion acceptor comprised within the second polypeptide.

FIG. 4 shows a schematic diagram of the production of a product from a reactant in accordance with the methods of the invention, wherein electrons and/or hydride ions are transferred from the reduced flavin cofactor to a molecular substrate (in this case shown in non-limiting form as O₂).

FIG. 5 shows a schematic diagram of the production of a product from a reactant in accordance with the methods of the invention wherein the first polypeptide and second polypeptide are attached together, for example in the form of a fusion protein or by being cross-linked together.

FIG. 6 shows a schematic of two-electron flavin reduction by Hyd1. H₂ oxidation at the [NiFe] active site provides 2 electrons that are transferred to the surface of the protein via FeS clusters. Figure prepared using PyMOL™ 2.3.4 (PDB: 6FPW).

FIG. 7 shows results of an activity assay for H₂-driven Hyd1 reduction of flavin measured by in situ UV-visible spectroscopy. A) Hyd1 reducing FMN. B) Hyd1 reducing FAD. C) Calculated [FMN] based on λₘₐₓ=445 nm (ε=12.50 mM⁻¹ cm⁻¹). D) Calculated [FAD] based on λₘₐₓ=450 nm (ε=11.30 mM⁻¹ cm⁻¹). Reaction conditions: General Procedure A in Tris-HCl buffer (50 mM, pH 8.0, 25° C.). Results are described in example 1.

FIG. 8A shows Hyd1-catalysed flavin reduction at different temperatures. Reaction conditions: General Procedure A in phosphate buffer (50 mM, pH 8.0). Conversion was calculated after 30 min using UV-visible spectroscopy. Results are described in example 1. FIG. 8B shows Hyd1-catalysed flavin reduction at different temperatures.

$$Conversion relative to standard = \frac{Conversion at temp}{Conversion at 25 ° C .} \times 100 \% .$$

The FMN 25-50° C. bars Conversion at 25° C. represent the average of relative conversions calculated from duplicate experiments, with the range represented as error bars. Reaction conditions: General Procedure A (Supporting Information, Example 1) in phosphate buffer (50 mM, pH 8.0). Conversion was calculated after 30 min using UV-visible spectroscopy.

FIG. 9 shows current applications and methods of flavin recycling.

FIGS. 10 to 12 show the results of control experiments, discussed in example 1. FIG. 10 shows background flavin reduction in absence of H₂. FIG. 11 shows background flavin reduction in absence of Hyd1. FIG. 12 shows background flavin reduction in absence of Hyd1.

FIG. 13 shows UV-visible spectra of FMN and FMNH₂ produced by Hyd1 under H₂ or sodium dithionite (gray). Results discussed in example 1.

FIG. 14 shows exemplary chiral-phase GC-FID traces of enzymatic H₂-driven reduction of ketoisophorone to (R)-levodione. Results discussed in example 1.

FIG. 15 shows exemplary GC-FID traces of enzymatic E. coli Hyd1 H₂-driven reduction of 4-phenyl-3-buten-2-one (5) reduction to 4-phenyl-2-butanone (6). Results discussed in example 2.

FIG. 16 shows exemplary GC-FID traces of enzymatic E. coli Hyd1 H₂-driven reduction of dimethyl itaconate (3) to dimethyl (R)-methyl succinate (4). Results discussed in example 2.

FIG. 17 shows ¹H NMR spectra of compound standards in H₂O/D₂O, run with water suppression. FIG. 17A shows full speactrum; B shows zoom-in of the aromatic proton region. Results described in example 3.

FIG. 18 shows activity assay results for E. coli Hyd2 catalysed reduction of flavins. Results described in example 4.

FIG. 19 shows activity assay results for Desulfovibrio vulgaris Miyazaki F catalysed reduction of flavins. Results described in example 4.

FIG. 20 shows the specific activity of E. coli Hyd1 for FAD reduction measured at different mixtures of water:solvent. A: measurements taken at different mixtures of water:DMSO. B: measurements taken at different mixtures of water:acetonitrile. Results described in example 5.

### DETAILED DESCRIPTION OF THE INVENTION

### Methods of the Invention

As described above, the invention provides a method of producing a reaction product, comprising:
i) contacting an oxidised flavin cofactor and molecular hydrogen (¹H₂) or an isotope thereof with a first polypeptide which is a hydrogenase enzyme or a functional fragment or derivative thereof under conditions such that the oxidised flavin cofactor is reduced to form a reduced flavin cofactor; and
ii) contacting the reduced flavin cofactor and a reactant with a second polypeptide which is an oxidoreductase or a functional fragment or derivative thereof under conditions such that the oxidised flavin cofactor is regenerated; and (b) the second polypeptide catalyses the formation of the reaction product from the reactant.

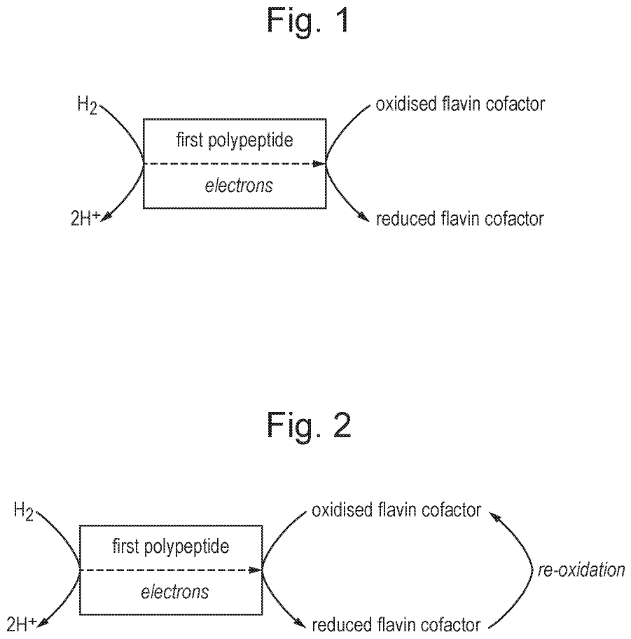

In the first step of the method above, an oxidised flavin cofactor and molecular hydrogen (¹H₂) or an isotope thereof are contacted with a first polypeptide. The first polypeptide typically oxidises the molecular hydrogen to produce protons and electrons. The first polypeptide is described in more detail herein. The electrons generated by the oxidation of the molecular hydrogen preferably reduce the oxidised flavin cofactor to form a reduced flavin cofactor. This is shown schematically in FIG. 1. Flavin cofactors suitable for use in the invention are described below.

Isotopes of molecular hydrogen suitable for use in the invention include ²H₂ and ³H₂. Mixed isotopes (e.g. ¹H²H and ¹H³H) are also embraced. Preferably, in the invention, the hydrogen is ¹H₂. It will be apparent that, as used herein, organic molecules such as glucose, formate, and ethanol, isopropanol, etc, are not sources of molecular hydrogen.

The molecular hydrogen is typically provided in the form of a gas. The gas may be mixed with an aqueous solution in which the first polypeptide and other reaction components such as the second polypeptide and reactant are present. At 1 bar H₂ the solubility of H₂ in water is 0.8 mM. In other words, by providing the hydrogen in the form of molecular hydrogen gas, the first polypeptide typically operates under concentrations of 0.8 mM hydrogen. Other pressures may also be used. For example, the gas pressure in the reaction vessel may be from 0.01 to about 100 bar, such as from 0.1 to 10 bar, e.g. from about 0.2 to about 5 bar, e.g. from 0.5 to 2 bar, such as approximately 1 bar.

The hydrogen may be provided as a mixture of hydrogen and other gases such as CO, CO₂, air, O₂, N₂, Ar, etc. When provided as a mixture, the mixture may comprise from about 0.1% to about 99% hydrogen, such as from 1% to about 95%, e.g. from about 2% to about 10% H₂.

The hydrogen used in the invention may be of any suitable purity. For example, hydrogen of 99% purity or greater (e.g. 99.9%, 99.99% or 99.999%) may be used when it is important to control impurity levels in the final product mixture. In other aspects, lower purity hydrogen may be used when it is not so important to control impurity levels in the final product mixture. For example, relatively low purity hydrogen may be provided in the form of “syngas”. Syngas produced by coal gasification generally is a mixture of 30 to 60% carbon monoxide, 25 to 30% hydrogen, 5 to 15% carbon dioxide, and 0 to 5% methane, and may optionally comprise lesser amount of other gases also.

For avoidance of doubt, the molecular hydrogen may also be provided in the form of a solution (e.g. an aqueous solution, e.g. comprising buffer salts as described in more detail here) in which molecular hydrogen is dissolved.

The molecular hydrogen may be provided from any suitable source, such as a gas cylinder. Alternatively, the molecular hydrogen or isotope thereof can be produced in situ e.g. by electrolysis of water.

In the second step of the method above, the reduced flavin cofactor generated in the first step and a reactant are contacted with a second polypeptide. The second polypeptide is described in more detail herein. Usually, electrons and/or hydride ions are transferred from the reduced flavin cofactor to an acceptor therefor. The acceptor may be comprised in the second polypeptide, for example the acceptor may comprise an active site of the second polypeptide or may comprise a prosthetic group comprised in the second polypeptide. The acceptor may comprise a molecular substrate. The molecular substrate is typically exogenous, i.e. is not part of the second polypeptide. Preferred substrates for use in this aspect of the invention include O₂.

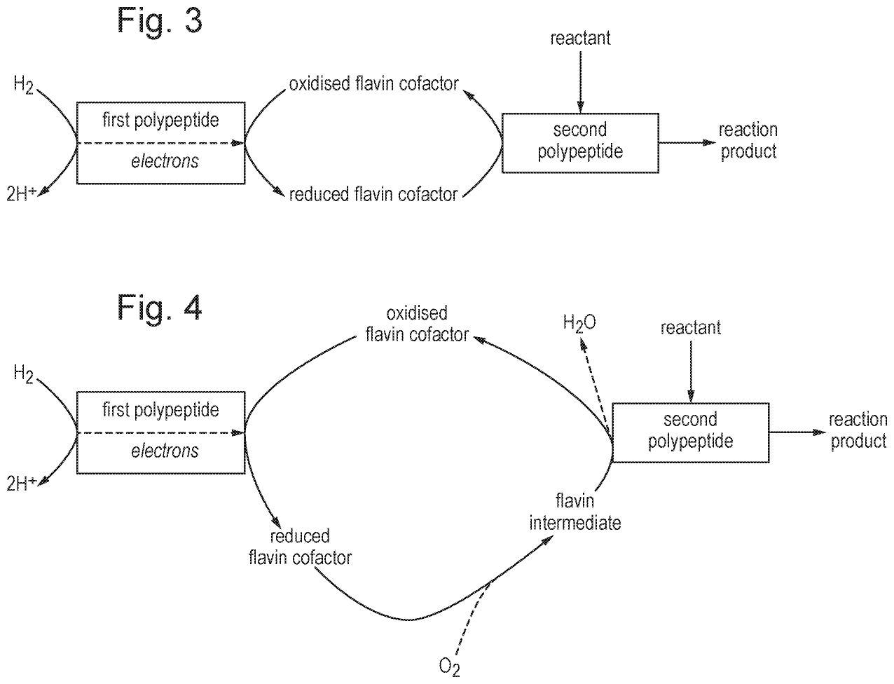

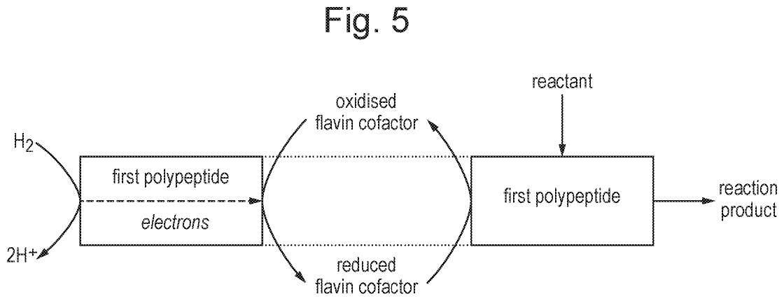

The transfer of electrons and/or hydride to the acceptor generates oxidised flavin cofactor. Accordingly, the oxidation of the flavin cofactor leads to the regeneration of the oxidised flavin cofactor used in the provided methods. The process is typically as shown schematically in FIGS. 2 and 3. The second polypeptide catalyses the formation of the reaction product from the reactant. Suitable reactants and the products thereby produced are described in more detail herein.

Accordingly, the method of producing a reaction product preferably comprises:
i) contacting an oxidised flavin cofactor and molecular hydrogen (¹H₂) or an isotope thereof with a first polypeptide which is a hydrogenase enzyme or a functional fragment or derivative thereof under conditions such that the first polypeptide oxidises the hydrogen to produce protons and electrons, and transfers the electrons to the oxidised flavin cofactor, thereby reducing the oxidised flavin cofactor to form a reduced flavin cofactor; and
ii) contacting the reduced flavin cofactor and a reactant with a second polypeptide which is an oxidoreductase or a functional fragment or derivative thereof under conditions such that (a) electrons are transferred from the reduced flavin cofactor to an electron acceptor and/or hydride ions are transferred from the reduced flavin cofactor to a hydride ion acceptor, (b) the oxidised flavin cofactor is regenerated; and (c) the second polypeptide catalyses the formation of the reaction product from the reactant.

In preferred embodiments of the invention, method steps (i) and (ii) are repeated multiple times thereby recycling the cofactor. For avoidance of doubt, by recycling the cofactor, it is meant that a single cofactor molecule can be reduced in a method of the invention from the oxidised form to a reduced form. The reduced cofactor can subsequently transfer electrons and/or a hydride ion to an electron acceptor and/or hydride acceptor as described above, thus oxidising the cofactor, which can be re-reduced as described. The repeated reduction and oxidation of the cofactor corresponds to recycling of the cofactor. The net result is that the cofactor itself is not spent.

In methods of the invention which involve recycling the cofactor, each cofactor molecule is typically recycled as defined herein at least 10 times, such as at least 50 times, e.g. at least 100 times, more preferably at least 1000 times e.g. at least 10,000 times or at least 100,000 times, such as at least 1,000,000 times. Accordingly, in methods of the invention, the turnover number (TN) is typically at least 10, such as at least 100, more preferably at least 1000 e.g. at least 10,000 or at least 100,000, such as at least 1,000,000. As those skilled in the art will appreciate, the TN indicates the number of moles of product generated per mole of cofactor, and is thus a measure of the number of times each cofactor molecule is used.

Enzyme turnover can be calculated in a number of ways. The Total Turnover Number (TTN, also known as the TON) is a measure of the number of moles of product per mole of enzyme (specifically per mole of the first polypeptide). As those skilled in the art will appreciate, the TTN thus indicates the number of times that the enzyme (i.e. the first polypeptide) has turned over. Preferably, the TTN is at least 10, such as at least 100, more preferably at least 1000 e.g. at least 10,000 or at least 100,000, such as at least 1,000,000, preferably at least 10⁷ such as at least 10⁸, e.g. at least 10⁹.

The Turnover Frequency (TOF) is a measure of the number of moles of product generated per second per mole of enzyme (first polypeptide) present. Accordingly, in methods of the invention for the production of a reduced cofactor, the TOF indicates the number of moles of reduced cofactor generated per second per mole of first polypeptide. Accordingly, the TOF is identified with the number of catalytic cycles undertaken by each enzyme molecule per second. Preferably, in the methods of the invention, the first polypeptide has a TOF of 0.1 to 1000 s⁻¹, more preferably 1 to 100 s⁻¹ such as from about 10 to about 50 s⁻¹.

### Flavin Cofactor

The methods of the invention involve the reduction and optional re-oxidation of an oxidised cofactor.

Preferably, the oxidised cofactor is a flavin cofactor. Flavin cofactors exist in the oxidised form (FI) and the reduced form (FIH2). The oxidized form acts as an electron acceptor, by being reduced. The reduced form, in turn, can act as a reducing agent, by being oxidized.

Preferably, the flavin cofactor is based on isoalloxazine. Typically, the flavin cofactor is a compound of Formula (I):

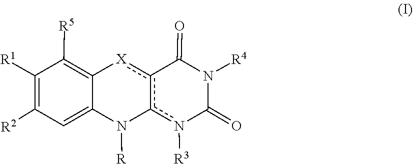

Typically, the compound of Formula (I) is a compound of Formula (Ia) or Formula (Ib), preferably (Ia):

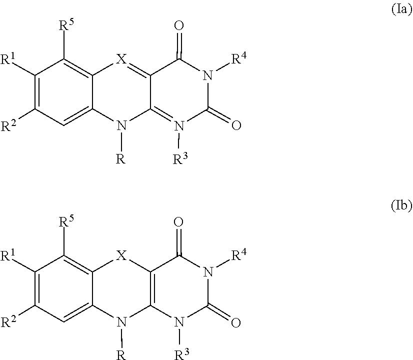

In formula (I), (Ia) and (Ib):
X is CH or N;
R¹ and R² are each independently selected from hydrogen, C₁₋₄ alkyl, halogen, —OH, —SH, nitro, —NR¹⁰R¹¹ and —N⁺R¹⁰R¹¹R¹²; and when R¹ and/or R² is an alkyl group the alkyl group is independently unsubstituted or is substituted by 1, 2 or 3 substituents independently selected from halogen, —OH, —SH, and nitro, —NR¹⁰R¹¹ and —N⁺R¹⁰R¹¹R¹²;
R⁴ is selected from hydrogen and C₁₋₄ alkyl; and when R⁴ is an alkyl group the alkyl group is unsubstituted or is substituted by 1, 2 or 3 substituents independently selected from halogen, —OH, and nitro, —NR¹⁰R¹¹ and —N⁺R¹⁰R¹¹R¹²;
R¹⁰, R¹¹ and R¹² are each independently H or methyl;
R⁵ is H or is an alkylene group and is attached to X to form an optionally substituted cyclic group;
R is an optionally substituted alkyl group;
and
R³ is absent or R³ is an alkylene group and is attached to R to form a cyclic group.

A C₁₋₄ alkyl group is a linear or branched alkyl group containing from 1 to 4 carbon atoms.

A C₁₋₄ alkyl group is often a C₁₋₃ alkyl group or a C₁₋₂ alkyl group. Examples of C₁ to C₄ alkyl groups include methyl, ethyl, n-propyl, iso-propyl, n-butyl, iso-butyl, sec-butyl, and tert-butyl, often methyl is preferred. Where two alkyl groups are present, the alkyl groups may be the same or different. An alkylene group is an unsubstituted or substituted bidentate moiety obtained by removing two hydrogen atoms from an alkane. The two hydrogen atoms may be removed from the same carbon atom or from different carbon atoms. Typically an alkylene group is a C₁ to C₄ alkylene group such as methylene, ethylene, n-propylene, iso-propylene, n-butylene, sec-butylene and tert-butylene. Where two alkylene groups are present, the alkylene groups may be the same or different. An alkyl or alkylene group may be unsubstituted or substituted, e.g. by one or more, e.g. 1, 2, 3 or 4 substituents selected from halogen, —OH, —SH, and nitro, —NR¹⁰R¹¹ and -N+R¹⁰R¹¹R¹², where R¹⁰⁻¹² are as defied herein. The substituents on a substituted alkyl or alkylene group are typically themselves unsubstituted. Where more than one substituent is present, these may be the same or different.

A cyclic group is typically a 4- to 10-membered carbocyclic group or a 4-10 membered heterocyclic group. A carbocyclic group is a cyclic hydrocarbon. A carbocyclic group may be saturated or partially unsaturated, but is typically saturated. A 4- to 10-membered partially unsaturated carbocyclic group is a cyclic hydrocarbon containing from 4 to 10 carbon atoms and containing 1 or 2, e.g. 1 double bond. Often, a 4- to 10-membered carbocyclic group is a 4- to 6-membered (e.g. 5- to 6-membered) carbocyclic group. Examples of 4- to 6-membered saturated carbocyclic groups include cyclobutyl, cyclopentyl and cyclohexyl groups. A 4- to 10-membered heterocyclic group is a cyclic group containing from 4 to 10 atoms selected from C, O, N and S in the ring, including at least one heteroatom, and typically one or two heteroatoms. The heteroatom or heteroatoms are typically selected from O, N, and S, most typically N. A heterocyclic group may be saturated or partially unsaturated. A 4- to 10-membered partially unsaturated heterocyclic group is a cyclic group containing from 4 to 10 atoms selected from C, O, N and S in the ring and containing 1 or 2, e.g. 1 double bond. Often, a 4- to 10-membered heterocyclic group is a monocyclic 4- to 6-membered heterocyclic group or a monocyclic 5- or 6-membered heterocyclic group, such as piperazine, piperidine, morpholine, 1,3-oxazinane, pyrrolidine, imidazolidine, and oxazolidine.

Preferably, X is N. Preferably, R¹ and R² are each independently selected from hydrogen and unsubstituted C₁₋₂ alkyl. Most preferably, R¹ and R² are each methyl.

Preferably, R³ is absent. When R³ is other than absent, the nitrogen atom to which R³ is attached is typically positively charged.

Preferably, R⁴ is hydrogen or unsubstituted C₁₋₂ alkyl. Most preferably, R⁴ is hydrogen.

Preferably, R⁵ is hydrogen or is attached to X to form a 6-membered heterocyclic group which is optionally substituted by 1 or 2 methyl groups. Most preferably, R⁵ is hydrogen.

Preferably, R is alkyl (preferably C₁₋₆alkyl) optionally substituted by one or more groups independently selected from —OH, —OC(O)—C₁₋₄alkyl (e.g. —OC(O)—CH₃), phenyl, and phosphate, wherein each phosphate group is optionally substituted. Preferred R moieties include —CH₂(CHOH)₃CH₂OH;
—CH₂(CHOH)₃CH₂OPO₃; —CH₂(CHOH)₃CH₂(OPO₃)₂-Adenine; —CH₂(CHOC(O)CH₃)₃CH₂ OC(O)CH₃;
—CH₂CH(CH₂CH₃)₂; and —CH₂CH(CH₂C6H₅)₂.

Preferably, therefore, X is N; R¹ and R² are each methyl; R⁴ is hydrogen; R³ is absent and R is alkyl substituted by one or more groups selected from —OH, —OC(O)—CH₃, phenyl and phosphate.

Preferably, the flavin cofactor is selected from flavin mononucleotide (FMN), flavin adenine dinucleotide (FAD), riboflavin, or a derivative thereof. The structures of riboflavin, flavin adenine dinucleotide (FAD), and flavin mononucleotide (FMN) in their respective oxidised forms are shown below.

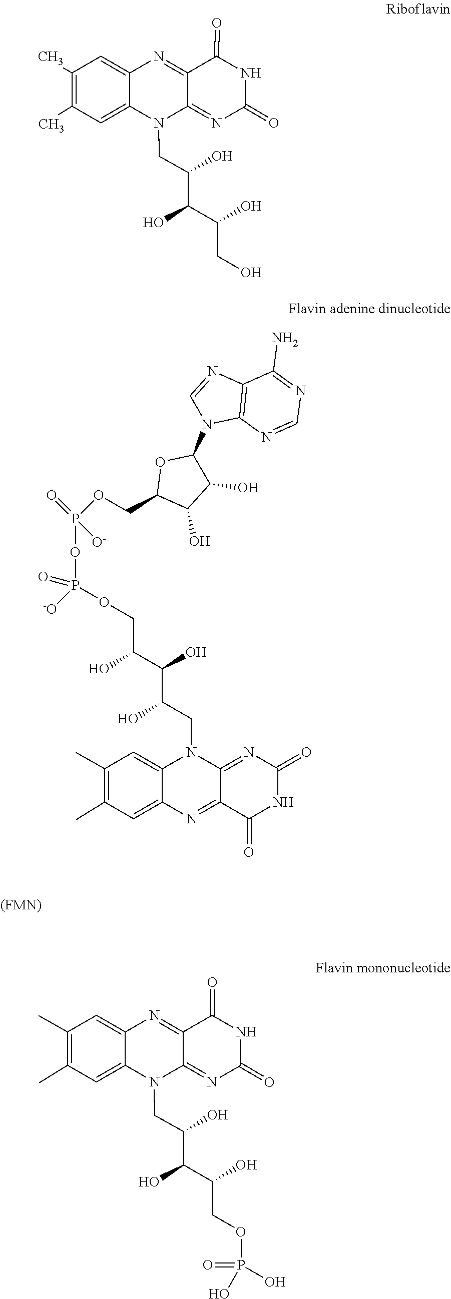

Derivatives of flavin cofactors may also be modified at the position corresponding to group R in formula (I). For example, in any of FMN, FAD and riboflavin the alkyl group attached to the isoalloxazine moiety may be modified, e.g. by modification of one or more of the —OH groups. Alternatively, in flavin cofactors which comprise a substituted phosphate group (as in FAD, for example), the phosphate group may be modified to alter the substituents thereon. Derivatives of flavin cofactors may also be modified at the positions corresponding to R¹, R², and R⁴ of Formula (I). Typically, such derivatives are modified in accordance with the definitions for Formula (I).

The reduction of the flavin cofactor occurs as shown in the reaction scheme below, in which X is depicted as N and R³ is depicted as being absent.

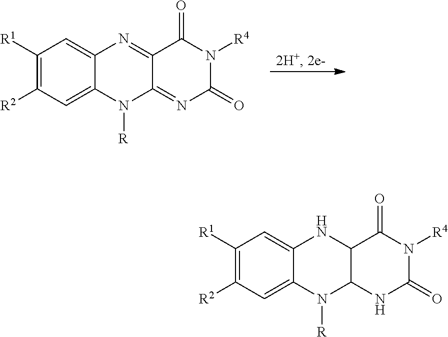

Preferably, in the invention, the flavin cofactor is selected from riboflavin, flavin adenine dinucleotide (FAD), and flavin mononucleotide (FMN). More preferably, the cofactor is flavin adenine dinucleotide (FAD) or a derivative thereof or flavin mononucleotide (FMN) or a derivative thereof. Most preferably the cofactor is flavin adenine dinucleotide (FAD) or flavin mononucleotide (FMN).

### First Polypeptide

In the invention, the first polypeptide is a hydrogenase enzyme or a functional fragment or derivative thereof. Any suitable hydrogenase can be used. The hydrogenase may comprise an active site comprising iron atoms (as in the [FeFe]-hydrogenases) or both nickel and iron atoms (as in the [NiFe]- and [NiFeSe]-hydrogenases). Preferably, the hydrogenase comprises an active site comprising both nickel and iron atoms. Suitable proteins are described below.

The first polypeptide is preferably selected or modified to catalyze H₂ oxidation close to the thermodynamic potential E° of the 2H⁺/H₂ couple (“E°(2H⁺/H₂)”) under the experimental conditions. (Those skilled in the art will appreciate that E°(2H⁺/H₂)=−0.413 V at 25° C., pH 7.0 and 1 bar H₂, and varies according to the Nernst equation). Preferably, the first polypeptide is selected or modified to catalyze H₂ or ^{x}H₂ oxidation at applied potentials of less than 100 mV more positive than E°(2H⁺/H₂); more preferably at applied potentials of less than 50 mV more positive than E°(2H⁺/H₂). Methods of determining the ability of a polypeptide to catalyze H₂ oxidation close to E°(2H⁺/H₂) under the experimental conditions at issue are routine for those skilled in the art and are, for example, described in Vincent et al, J. Am. Chem. Soc. (2005) 127, 18179-18189.

In the invention, the first polypeptide typically transfers the electrons to the oxidised flavin cofactor via an intramolecular electronically-conducting pathway. In other words, the electron transfer from the hydrogen electron source to the flavin cofactor is a direct electron transfer. While the invention does embrace the use of electron mediators (e.g. redox active dyes such as methyl or benzyl viologen) to mediate electron transfer from the hydrogen electron source (e.g. from the first polypeptide) to the flavin cofactor, the electron transfer is typically not mediated by electron transfer agents such as mediators, e.g. is typically not mediated by a redox active dye such as methyl or benzyl viologen. Typically, the intramolecular electronically-conducting pathway comprises a series of [FeS] clusters. As those skilled in the art will appreciate, [FeS]-clusters include [3Fe4S] and [4Fe4S] clusters.

Without being bound by theory, the inventors consider that the reduction of the oxidised flavin cofactor preferably takes place at an [FeS] cluster within the first polypeptide, preferably at the distal [FeS] cluster. The notation “distal” in this context is routine in the art. For a protein which contains an active site and a chain or series of [FeS] clusters, the proximal cluster is the [FeS] cluster at closest proximity to the active site. The distal cluster is the [FeS] cluster closest to a solvent-accessible surface of the protein, and thus furthest away from the active site. [FeS] clusters between the proximal and distal clusters are referred to as medial clusters. The distal cluster is often solvent accessible. Sometimes, the distal cluster can be accessed by the oxidised flavin cofactor.

Preferably, in the invention, the first polypeptide does not comprise a native flavin active site for NAD(P)⁺ reduction. Some known hydrogenases do comprise such an active site. Hydrogenase enzymes which do comprise a native flavin active site for NAD(P)+ reduction include the soluble hydrogenase (SH) enzymes from R. eutropha, Rhodococcus opacus, Hydrogenophilus thermoluteolus and Pyrococcus furiosus. Accordingly, the first polypeptide is typically not selected from the soluble hydrogenase (SH) enzymes from R. eutropha, Rhodococcus opacus, Hydrogenophilus thermoluteolus and Pyrococcus furiosus. Native flavin sites are also sometimes referred to as flavin prosthetic groups. Accordingly, the first polypeptide preferably does not comprise a flavin prosthetic group. Without being bound by theory, it is believed that hydrogenases lacking such groups typically have increased stability compared to hydrogenases comprising such prosthetic groups. Examples of hydrogenases lacking a flavin prosthetic group include Escherichia coli hydrogenase 1 (SEQ ID NOs:1-2), Escherichia coli hydrogenase 2 (SEQ ID NOs:3-4), Ralstonia eutropha membrane-bound hydrogenase (SEQ ID NOs: 5-7), Ralstonia eutropha regulatory hydrogenase (SEQ ID NOs:8-9), Aquifex aeolicus hydrogenase 1 (SEQ ID NOs:10-11), and Hydrogenovibrio marinus membrane-bound hydrogenase (SEQ ID NOs: 12-13).

It is a surprising finding of the present invention that reduction of oxidised flavin cofactors can be catalysed by hydrogenase enzymes which do not comprise a native flavin prosthetic group. Without being bound by theory, it is believed that such enzymes typically operate in vivo by shuttling electrons into the quinone pool of the parent organism and as such there is no requirement in vivo for a redox cofactor such as flavin to be accessible to the electron transfer pathway in the protein. It was previously unknown that electrons could be passed between the active site of such enzymes and an exogenous cofactor such as a flavin.

Preferably, in the invention, the first polypeptide is an uptake hydrogenase or a hydrogen-sensing hydrogenase. Uptake hydrogenases are used by organisms in vivo to generate energy by oxidation of molecular hydrogen in their environment. In vivo, they link oxidation of H₂ to reduction of anaerobic acceptors such as nitrate and sulfate, or O₂. Typically, uptake hydrogenases comprise a signal peptide (often of length from about 30 to about 60 amino acid residues) at the N terminus of the small subunit. Typically, the signal peptide comprises a [DENST]RRxFxK motif Hydrogen sensing hydrogenases (also known as regulatory hydrogenases) are used by organisms in vivo to sense hydrogen levels in order to control biosynthesis of uptake hydrogenases in response to H₂. Regulatory hydrogenases typically do not comprise the signal peptide characteristic of uptake hydrogenases. Regulatory hydrogenases are often insensitive to O₂.

Preferably, the hydrogenase is selected or modified to be oxygen tolerant. Oxygen tolerant hydrogenases are capable of oxidising H₂ or H₂ in the presence of oxygen, such as in the presence of at least 0.01% O₂, preferably at least 0.1% O₂, more preferably at least 1% O₂, such as at least 5% O₂, e.g. at least 10% O₂ such as at least 20% O₂ or more whilst retaining at least 1%, preferably at least 5%, such as at least 10%, preferably at least 20%, more preferably at least 50% such as at least 80% e.g. at least 90% preferably at least 95% e.g. at least 99% of their H₂-oxidation activity under anaerobic conditions. Various oxygen-tolerant hydrogenases are known to those skilled in the art.

Preferably, in the invention, the first polypeptide is a hydrogenase of class 1 or 2b. References to hydrogenase classes such as class 1 and class 2b refer to the established Vignais classification scheme described by Vignais and Billoud, Chem. Rev. 2007, 107, 4206-4272, which is known to those skilled in the art. The hydrogenase may be any of the hydrogenases of class 1 or class 2b listed in Vignais and Billoud, Chem. Rev. 2007, 107, 4206-4272, the contents of which are incorporated by reference.

Preferably, the first polypeptide is selected from or comprises:
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Ralstonia eutropha regulatory hydrogenase moiety (SEQ ID NOs: 8 and/or 9) or an amino acid sequence having at least 60% homology therewith;
v) the amino acid sequence of Aquifex aeolicus hydrogenase 1 (SEQ ID NO:10 and/or 11) or an amino acid sequence having at least 60% homology therewith;
vi) the amino acid sequence of Hydrogenovibrio marinus hydrogenase (SEQ ID NOs: 12 and/or 13) or an amino acid sequence having at least 60% homology therewith;
vii) the amino acid sequence of Thiocapsa roseopersicina hydrogenase (SEQ ID NOs: 14 and 15) or an amino acid sequence having at least 60% homology therewith;
viii) the amino acid sequence of Alteromonas macleodii hydrogenase (SEQ ID NOs: 16 and/or 17) or an amino acid sequence having at least 60% homology therewith;
ix) the amino acid sequence of Allochromatium vinosum membrane bound hydrogenase (SEQ ID NOs: 18 and/or 19) or an amino acid sequence having at least 60% homology therewith;
x) the amino acid sequence of Salmonella enterica serovar Typhimurium LT2 nickel-iron hydrogenase 5 (SEQ ID NO: 20 and/or 21) or an amino acid sequence having at least 60% homology therewith; or
xi) the amino acid sequence of Desulfovibrio vulgaris Miyazaki F hydrogenase (SEQ ID NO: 23 and/or 24) or an amino acid sequence having at least 60% homology therewith;
   or a functional fragment, derivative or variant thereof.

Preferably, the first polypeptide is selected from or comprises:
i) the amino acid sequence of Escherichia coli hydrogenase 1 (SEQ ID NOs:1 and/or 2) or an amino acid sequence having at least 60% homology therewith; ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Ralstonia eutropha regulatory hydrogenase moiety (SEQ ID NOs: 8 and/or 9) or an amino acid sequence having at least 60% homology therewith;
v) the amino acid sequence of Aquifex aeolicus hydrogenase 1 (SEQ ID NO:10 and/or 11) or an amino acid sequence having at least 60% homology therewith;
vi) the amino acid sequence of Hydrogenovibrio marinus hydrogenase (SEQ ID NOs: 12 and/or 13) or an amino acid sequence having at least 60% homology therewith;
vii) the amino acid sequence of Thiocapsa roseopersicina hydrogenase (SEQ ID NOs: 14 and 15) or an amino acid sequence having at least 60% homology therewith;
viii) the amino acid sequence of Alteromonas macleodii hydrogenase (SEQ ID NOs: 16 and/or 17) or an amino acid sequence having at least 60% homology therewith;
ix) the amino acid sequence of Allochromatium vinosum membrane bound hydrogenase (SEQ ID NOs: 18 and/or 19) or an amino acid sequence having at least 60% homology therewith; or
x) the amino acid sequence of Salmonella enterica serovar Typhimurium LT2 nickel-iron hydrogenase 5 (SEQ ID NO: 20 and/or 21) or an amino acid sequence having at least 60% homology therewith;
   or a functional fragment, derivative or variant thereof.

More preferably, the first polypeptide is selected from or comprises:
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Aquifex aeolicus hydrogenase 1 (SEQ ID NO:10 and/or 11) or an amino acid sequence having at least 60% homology therewith;
v) the amino acid sequence of Hydrogenovibrio marinus hydrogenase (SEQ ID NOs: 12 and/or 13) or an amino acid sequence having at least 60% homology therewith;
vi) the amino acid sequence of Alteromonas macleodii hydrogenase (SEQ ID NOs: 16 and/or 17) or an amino acid sequence having at least 60% homology therewith; or
vii) the amino acid sequence of Salmonella enterica serovar Typhimurium LT2 nickel-iron hydrogenase 5 (SEQ ID NO: 20 and/or 21) or an amino acid sequence having at least 60% homology therewith;
   or a functional fragment, derivative or variant thereof.

Sometimes, the first polypeptide is selected from or comprises:
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith; or
iii) the amino acid sequence of Desulfovibrio vulgaris Miyazaki F hydrogenase (SEQ ID NO: 23 and/or 24) or an amino acid sequence having at least 60% homology therewith;
   or a functional fragment, derivative or variant thereof.

Most preferably, the first polypeptide comprises the amino acid sequence of Escherichia coli hydrogenase 1 (SEQ ID NOs:1 and/or 2) or an amino acid sequence having at least 60% homology therewith; or a functional fragment, derivative or variant thereof.

Preferably, when the first polypeptide comprises or consists of one or more amino acid sequences having at least 60% homology with a specified sequence, each amino acid sequence independently has at least 70%, such as at least 80%, more preferably at least 90%, e.g. at least 95%, preferably at least 97%, such as at least 98%, preferably at least 99% homology with the specified sequence. More preferably, each amino acid sequence independently has at least 70%, such as at least 80%, more preferably at least 90%, e.g. at least 95%, preferably at least 97%, such as at least 98%, preferably at least 99% identity with the specified sequence. For avoidance of doubt, if the first polypeptide comprises two or more amino acid sequences, the percentage homology of each of the two or more sequences with respect to their respective specified sequences can be the same or different, preferably the same. Percentage homology and/or percentage identity are each preferably determined across the length of the specified reference sequence as described herein.

It will be apparent to the skilled person that the first polypeptide may either be a single polypeptide or may comprise multiple polypeptides. The first polypeptide may also be a portion such as one or more domains of a multidomain polypeptide. Those skilled in the art will appreciate that hydrogenase enzymes typically comprise two or more subunits. As used herein, the term “first polypeptide” relates to one or more of the subunits of the relevant protein. For example, when the first polypeptide is Escherichia coli hydrogenase 1 (SEQ ID NOs:1 and/or 2), the first polypeptide may comprise (i) SEQ ID NO: 1 but not SEQ ID NO: 2; (ii) SEQ ID NO: 2 but not SEQ ID NO: 1; or (iii) both SEQ ID NO: 1 and SEQ ID NO: 2. When the first polypeptide is Escherichia coli hydrogenase 2 (SEQ ID NOs:3 and/or 4), the first polypeptide may comprise (i) SEQ ID NO: 3 but not SEQ ID NO: 4; (ii) SEQ ID NO: 4 but not SEQ ID NO: 3; or (iii) both SEQ ID NO: 3 and SEQ ID NO: 4. When the first polypeptide is Desulfovibrio vulgaris Miyazaki F hydrogenase (SEQ ID NOs: 23 and/or 24), the first polypeptide may comprise (i) SEQ ID NO: 23 but not SEQ ID NO: 24; (ii) SEQ ID NO: 24 but not SEQ ID NO: 23; or (iii) both SEQ ID NO: 23 and SEQ ID NO: 24. Typically, when the first polypeptide is a hydrogenase enzyme having two or more subunits, the first polypeptide comprises said two or more subunits.

Furthermore, the first polypeptide may be used in the invention in the form of a monomer or a multimer. For example, when the first polypeptide comprises a hydrogenase which can exist in a monomeric or dimeric form, the first polypeptide used in the invention can be provided in the form of the monomer or the dimer. For example, Escherichia coli hydrogenase 1 may be purified either as a dimer or a monomer or a mixture thereof. When the first polypeptide comprises Escherichia coli hydrogenase 1 (i.e. SEQ ID NOs: 1 and/or 2) the first polypeptide may be provided as a monomer (1×SEQ ID NO: 1 and/or 1×SEQ ID NO 2) or as a dimer (2×SEQ ID NO: 1 and/or 2×SEQ ID NO: 2), or as a mixture thereof. When the first polypeptide is provided as a mixture of a monomer and dimer, the mixture typically contains from about 1% to about 99% of the monomer and from about 99% to about 1% of the dimer. Sometimes, the amount of monomer and dimer may be approximately similar, and the first polypeptide may thus comprise from about 30% to about 70% monomer and from about 70% to about 30% dimer, such as from about 40% to about 60% monomer and from about 60% to about 40% dimer. Sometimes, the first polypeptide comprises from about 1 to about 10% monomer/about 90% to about 99% dimer, e.g. from about 1% to about 5% monomer/about 95% to about 99% dimer. Sometimes, the first polypeptide comprises from about 1 to about 10% dimer/about 90% to about 99% monomer, e.g. from about 1% to about 5% dimer/about 95% to about 99% monomer.

It will also be apparent to those skilled in the art that the first polypeptide may comprise associated proteins which may for example be co-purified with the first polypeptide. For example, when the first polypeptide comprises the amino acid sequence of SEQ ID NO: 1 and/or 2 (or a functional fragment, derivative or variant thereof), the first polypeptide may further comprise a native cytochrome electron transfer partner such as the cytochrome of SEQ ID NO: 22 (or a functional fragment, derivative or variant thereof). Thus, in embodiments of the invention in which the first polypeptide comprises SEQ ID NO: 1 and/or 2 (or a functional fragment, derivative or variant thereof), the first polypeptide may also comprise SEQ ID NO: 22 (or a functional fragment, derivative or variant thereof).

### Second Polypeptide

In inventive methods which comprise the use of a second polypeptide to catalyse the conversion or a reagent to a product, any suitable second polypeptide may be used. Typically, electrons are transferred from the reduced flavin cofactor to an electron acceptor and/or hydride ions are transferred from the reduced flavin cofactor to a hydride ion acceptor, thereby regenerating the oxidised flavin cofactor. Usually, in the invention, electrons are transferred from the reduced flavin cofactor to an electron acceptor.

In one embodiment, the second polypeptide comprises an electron acceptor and/or hydride ion acceptor. The acceptor group may consist of or comprise a prosthetic group or active site within the second polypeptide. Thus, in this embodiment, the second polypeptide typically comprises a prosthetic group for oxidising the reduced flavin cofactor. The second polypeptide typically comprises a flavin prosthetic group. Preferably, the flavin group is an FAD (flavin adenine dinucleotide) or FMN (flavin mononucleotide) group. This embodiment is shown schematically in FIG. 3.

In another embodiment, electrons and/or hydride are transferred at the second polypeptide from the reduced cofactor to a molecular substrate. The molecular substrate is preferably an exogenous substrate; i.e. it is not part of the second polypeptide. Examples of molecular substrates for use in the invention include O₂. For example, when O₂ is used as the molecular substrate, the reduced cofactor may form an oxidised cofactor comprising a product of the O₂ reduction such as a peroxo group. For example, an oxidised cofactor comprising a peroxo group obtained from O₂ reduction may be of the form:

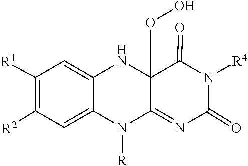

(with reference to Formula (I) above, in which X is depicted as N and R³ is depicted as being absent).

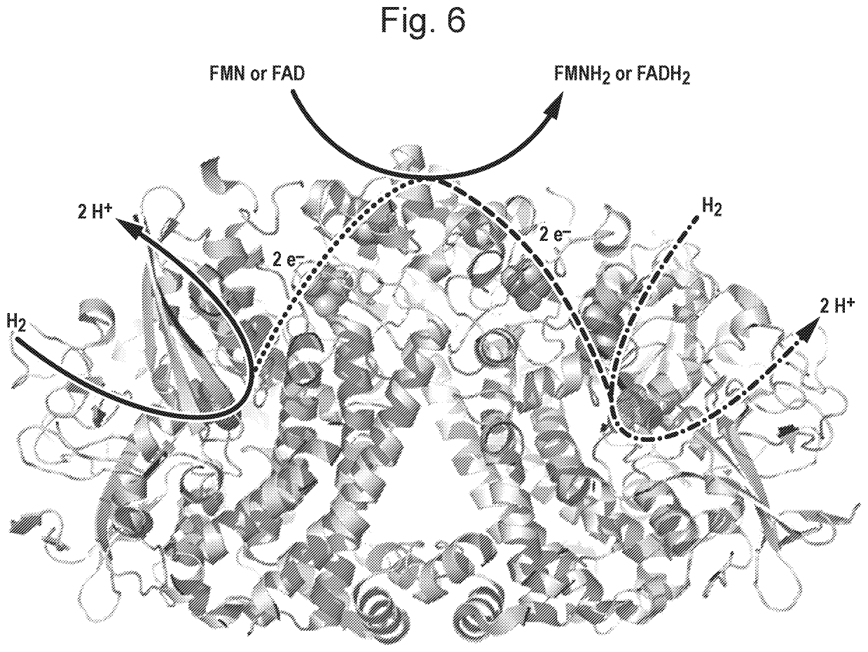

The second polypeptide may catalyse the reaction of the product of the molecular substrate reduction (e.g. the peroxo group) with the reagent in order to form the product. When O₂ is used as the molecular substrate, water is typically produced as a by-product. This embodiment is shown schematically as FIG. 4.

In another embodiment, electrons and/or hydride are transferred from the reduced cofactor to a molecular substrate. The molecular substrate is preferably an exogenous substrate; i.e. it is not part of the second polypeptide. Examples of molecular substrates for use in the invention include O₂. For example, when O₂ is used as the molecular substrate, the reduced cofactor may form an oxidised cofactor comprising a product of the O₂ reduction such as a peroxo group, e.g. as discussed above. The peroxy oxidised cofactor may release H₂O₂ to regenerate the oxidised cofactor. The H₂O₂ thus released may be used to convert a reactant to a product in accordance with the invention. Preferably, the second polypeptide catalyses the conversion of the reactant to the product.

Typically, the second polypeptide is a flavin-accepting oxidoreductase, or a functional fragment, derivative or variant thereof. In one embodiment, a flavin-accepting oxidoreductase is an enzyme which is facultatively capable of abstracting electrons and/or hydride from a reduced flavin cofactor. Those skilled in the art will appreciate that in consuming the reduced flavin, oxidised flavin is obtained. Thus, the flavin cofactor is not destroyed by the second polypeptide. In another embodiment, a flavin-accepting oxidoreductase is an enzyme which is facultatively capable of catalysing the reaction of an oxidised form of a flavin cofactor (e.g. that obtained by oxidation of a reduced cofactor with a molecular substrate such as O₂) with a reagent, thus forming a product and regenerating the oxidised flavin. Thus, the flavin cofactor is not destroyed by the second polypeptide.

More typically, the second polypeptide is a flavin-dependent oxidoreductase, or a functional fragment, derivative or variant thereof. A flavin-dependent oxidoreductase as used herein requires flavin cofactors such as FMN and FAD. Thus, a flavin-accepting oxidoreductase is capable of utilising various reduced cofactors including flavins as an electron/hydride source. A flavin-dependent oxidoreductase is only capable of using flavins as an electron/hydride source.

It will be apparent to the skilled person that the second polypeptide may either be a single polypeptide or may comprise multiple polypeptides, e.g. additional peptides in addition to the flavin-accepting or flavin-dependent oxidoreductase. The second polypeptide may also be a portion such as one or more domains of a multidomain polypeptide.

Preferably, the second polypeptide is a monooxygenase, halogenase or ene-reductase, or a functional fragment, derivative or variant thereof. More preferably, the second polypeptide is a flavin monooxygenase, a flavin halogenase, a flavin ene-reductase, a nitro reductase, a peroxidase or a haloperoxidase. Still more preferably, the second polypeptide is a flavin monooxygenase, a flavin halogenase, or a flavin ene-reductase. Preferably, the second polypeptide is of the “Old Yellow Enzyme” (OYE) type. OYEs are flavin-dependent redox enzymes and include e.g. OYE ene reductases.

Such enzymes catalyse commercially useful reactions. For example, halogenases catalyse chlorination, bromination, and iodination reactions. Ene-reductase catalyse reactions such as alkene reduction and nitro reduction. Monooxygenase enzymes catalyse reactions such as epoxidations, hydroxylations, and Baeyer-Villiger oxidations. Nitro reductases catalyse reactions such as the reduction of aromatic nitro groups and quinones. Peroxidases catalyse reaction such as O atom insertion reactions. Haloperoxidases catalyse reactions such as the conversion of C—H groups to C—Y groups (wherein Y is a halogen). Such reactions are wide in utility, including in natural product synthesis, biodegradation of environmental pollutants, and non-native light-driven reactions.

Often, the second enzyme is selected from Enzyme Commission (EC) classes 1.1.98.; 1.3.1.; 1.5.1.; 1.6.99.; 1.7.1.; 1.7.99; 1.11.1.; 1.11.2.; 1.14.14.; and 1.14.99.; or a functional fragment, derivative or variant thereof.

Preferably, the second polypeptide is selected from Enzyme Commission (EC) classes 1.1.98.; 1.5.1.; 1.6.99.; 1.7.99.; 1.14.14.; and 1.14.99.; 1.3.1; 1.11.1.; 1.11.2.; or a functional fragment, derivative or variant thereof; more preferably the second polypeptide is selected from Enzyme Commission (EC) classes 1.1.98.; 1.5.1.; 1.6.99.; 1.7.99.; 1.14.14.; and 1.14.99.; or a functional fragment, derivative or variant thereof.

More preferably, the second polypeptide is selected from or comprises
i) the amino acid sequence of Chromate Reductase, ‘TsOYE’ from Thermus scotuductus (SEQ ID NO:31) or an amino acid sequence having at least 60% homology therewith;
ii) the amino acid sequence of the NADPH Dehydrogenase 1, ‘OYE-1’, Saccharomyces pastorianus (SEQ ID NO:32) or an amino acid sequence having at least 60% homology therewith;
iii) the amino acid sequence of NADPH Dehydrogenase 2, ‘OYE-2’, Saccharomyces cerevisiae strain ATCC 204508 S288c (SEQ ID NO:33) or an amino acid sequence having at least 60% homology therewith;
iv) the amino acid sequence of NADPH Dehydrogenase, ‘YqjM’, Bacillus subtilis (SEQ ID NO:34) or an amino acid sequence having at least 60% homology therewith;
v) the amino acid sequence of Xenobiotic Reductase A, ‘XenA’, Pseudomonas putida (SEQ ID NO:35) or an amino acid sequence having at least 60% homology therewith;
vi) the amino acid sequence of NADPH dehydrogenase, ‘FOYE-1’, ‘Ferrovum’ strain JA12 (SEQ ID NO:36) or an amino acid sequence having at least 60% homology therewith;
vii) the amino acid sequence of Oxidored_FMN domain-containing protein, ‘MgER’, Meyerozyma guilliermondii (SEQ ID NO:37) or an amino acid sequence having at least 60% homology therewith;
viii) the amino acid sequence of Oxidored_FMN domain-containing protein, ‘CER’, Clavispora (Candida) lusitaniae (SEQ ID NO: 38) or an amino acid sequence having at least 60% homology therewith;
ix) the amino acid sequence of Tryptophan 2-Halogenase, ‘CmdE’, Chondromyces crocatus, (SEQ ID NO:39) or an amino acid sequence having at least 60% homology therewith;
x) the amino acid sequence of Tryptophan 5-Halogenase, ‘PyrH’, Streptomyces rugosporus (SEQ ID NO:40) or an amino acid sequence having at least 60% homology therewith;
xi) the amino acid sequence of Flavin-Dependent Tryptophan Halogenase, ‘RebH’, Lentzea aerocolonigenes (Lechevalieria aerocolonigenes) (Saccharothrix aerocolonigenes), (SEQ ID NO:41) or an amino acid sequence having at least 60% homology therewith;
xii) the amino acid sequence of Flavin-Dependent Tryptophan Halogenase, ‘PrnA’, Pseudomonas fluorescens (SEQ ID NO:42) or an amino acid sequence having at least 60% homology therewith;
xiii) the amino acid sequence of Thermophilic Tryptophan Halogenase, ‘Th-Hal’, Streptomyces violaceusnige (SEQ ID NO:43) or an amino acid sequence having at least 60% homology therewith;
xiv) the amino acid sequence of Tryptophan 6-Halogenase, ‘SttH’, Streptomyces toxytricini (SEQ ID NO: 44) or an amino acid sequence having at least 60% homology therewith;
xv) the amino acid sequence of KtzQ, ‘KtzQ’, Kutzneria sp. 744 (SEQ ID NO:45) or an amino acid sequence having at least 60% homology therewith;
xvi) the amino acid sequence of Monodechloroaminopyrrolnitrin halogenase, ‘PrnC’, Pseudomonas fluorescens (SEQ ID NO:46) or an amino acid sequence having at least 60% homology therewith;
xvii) the amino acid sequence of FADH₂-dependent halogenase, ‘PltA’, Pseudomonasprotegens Pf-5 (SEQ ID NO:47) or an amino acid sequence having at least 60% homology therewith;
xviii) the amino acid sequence of Halogenase, ‘PltM’, Pseudomonas fluorescens (strain ATCC BAA-477 NRRL B-23932 Pf-5) (SEQ ID NO:48) or an amino acid sequence having at least 60% homology therewith;
xix) the amino acid sequence of Flavin-Dependent Halogenase, ‘Clz5’, Streptomyces sp. CNH-287 (SEQ ID NO:49) or an amino acid sequence having at least 60% homology therewith;
xx) the amino acid sequence of Pyrrole Halogenase, ‘Bmp2’, Pseudoalteromonas piscicida (SEQ ID NO:50) or an amino acid sequence having at least 60% homology therewith;
xxi) the amino acid sequence of Non-Heme Halogenase, ‘Rdc2’, Metacordyceps chlamydosporia (Pochonia chlamydosporia) (SEQ ID NO:51) or an amino acid sequence having at least 60% homology therewith;
xxii) the amino acid sequence of Tryptophan 6-Halogenase, ‘BorH’, uncultured bacteria (SEQ ID NO:52) or an amino acid sequence having at least 60% homology therewith;
xxiii) the amino acid sequence of Styrene Monooxygenase, ‘StyA’, Pseudomonas sp. (SEQ ID NO:53) or an amino acid sequence having at least 60% homology therewith;
xxiv) the amino acid sequence of 4-Nitrophenol 2-Monooxygenase Oxygenase Component, ‘PheA1’, Rhodococcus erythropolis (Arthrobacter picolinophilus) (SEQ ID NO:54) or an amino acid sequence having at least 60% homology therewith;
xxv) the amino acid sequence of 4-Hydroxyphenylacetate 3-Monooxygenase Oxygenase Component, ‘HpaB’, Klebsiella oxytoca (SEQ ID NO:55) or an amino acid sequence having at least 60% homology therewith;
xxvi) the amino acid sequence of Chlorophenol Monooxygenase, ‘HadA’, Ralstonia pickettii (Burkholderia pickettii) (SEQ ID NO:56) or an amino acid sequence having at least 60% homology therewith;
xxvii) the amino acid sequence of Tetrachlorobenzoquinone Reductase, ‘PcpD’, Sphingobium chlorophenolicum (SEQ ID NO:57) or an amino acid sequence having at least 60% homology therewith;
xxviii) the amino acid sequence of 2-Methyl-6-ethyl-4-monooxygenase Oxygenase Component, ‘MeaX’, Sphingobium baderi (SEQ ID NO:58) or an amino acid sequence having at least 60% homology therewith;
xxix) the amino acid sequence of Alkanesulfonate Monooxygenase, ‘SsuD’, Escherichia coli (strain K12) (SEQ ID NO:59) or an amino acid sequence having at least 60% homology therewith;
xxx) the amino acid sequence of p-Hydroxyphenylacetate 3-Hydroxylase, Oxygenase Component, ‘C2-HpaH’, Acinetobacter baumannii (SEQ ID NO:60) or an amino acid sequence having at least 60% homology therewith; xxxi) the amino acid sequence of FADH(2)-Dependent Monooxygenase, ‘TftD’, Burkholderia cepacia (Pseudomonas cepacia) (SEQ ID NO:61) or an amino acid sequence having at least 60% homology therewith;
xxxii) the amino acid sequence of 4-Nitrophenol 2-Monooxygenase, Oxygenase Component, ‘NphA1’, Rhodococcus sp. (SEQ ID NO:62) or an amino acid sequence having at least 60% homology therewith;
xxxiii) the amino acid sequence of Putative dehydrogenase/oxygenase subunit, ‘VpStyA1’, Variovorax paradoxus (strain EPS) (SEQ ID NO:63) or an amino acid sequence having at least 60% homology therewith;
xxxiv) the amino acid sequence of Oxygenase, ‘RoIndA1’ {from styA1 gene}, Rhodococcus opacus (Nocardia opaca) (SEQ ID NO:64) or an amino acid sequence having at least 60% homology therewith;
xxxv) the amino acid sequence of Smoa_sbd domain-containing protein, ‘AbIndA’, Acinetobacter baylyi (strain ATCC 33305 BD413 ADP1) (SEQ ID NO:65) or an amino acid sequence having at least 60% homology therewith;
xxxvi) the amino acid sequence of 2,5-Diketocamphane 1,2-Monooxygenase 1, ‘CamP’, Pseudomonas putida (Arthrobacter siderocapsulatus) (SEQ ID NO:66) or an amino acid sequence having at least 60% homology therewith; xxxvii) the amino acid sequence of 3,6-Diketocamphane 1,6-Monooxygenase, ‘CamE36’, Pseudomonas putida (Arthrobacter siderocapsulatus), (SEQ ID NO:67) or an amino acid sequence having at least 60% homology therewith; xxxviii) the amino acid sequence of Alkanal monooxygenase, alpha and beta chain, ‘LuxAB’, Vibrio harveyi (Beneckea harveyi) (SEQ ID NOs:68 and 69) or an amino acid sequence having at least 60% homology therewith;
xxxix) the amino acid sequence of Alkanal monooxygenase, alpha and beta chain, ‘LuxAB’, Photorhabdus luminescens (Xenorhabdus luminescens) (SEQ ID NOs:70 and 71) or an amino acid sequence having at least 60% homology therewith;
xl) the amino acid sequence of Alkane Monooxygenase, ‘LadA’, Geobacillus thermodenitrificans (SEQ ID NO:72) or an amino acid sequence having at least 60% homology therewith;
xli) the amino acid sequence of EDTA Monooxygenase, ‘EmoA’, Chelativorans multitrophicus (SEQ ID NO:73) or an amino acid sequence having at least 60% homology therewith;
xlii) the amino acid sequence of Isobutylamine N-hydroxylase, ‘IBAH’, Streptomyces viridifaciens (SEQ ID NO:74) or an amino acid sequence having at least 60% homology therewith;
xliii) the amino acid sequence of ActVA 6 Protein, ‘ActVA-Orf6’, Streptomyces coelicolor, (SEQ ID NO:75) or an amino acid sequence having at least 60% homology therewith;
xliv) the amino acid sequence of Pyrimidine Monooxygenase, ‘RutA’, Escherichia coli (strain K12) (SEQ ID NO:76) or an amino acid sequence having at least 60% homology therewith;
xlv) the amino acid sequence of p-Hydroxyphenylacetate 2-Hydroxylase Reductase Component, ‘C1-HpaH’, Acinetobacter baumannii (SEQ ID NO:77) or an amino acid sequence having at least 60% homology therewith;
xlvi) the amino acid sequence of FMN_red Domain-Containing Protein, ‘YdhA’, Bacillus subtilis subsp. natto (strain BEST195) (SEQ ID NO:78) or an amino acid sequence having at least 60% homology therewith;
xlvii) the amino acid sequence of NAD(P)H-Flavin Reductase, ‘Fre’, Escherichia coli (strain K12) (SEQ ID NO:79) or an amino acid sequence having at least 60% homology therewith;
xlviii) the amino acid sequence of 4-hydroxyphenylacetate 3-monooxygenase reductase component, ‘HpaC’, Escherichia coli (SEQ ID NO:80) or an amino acid sequence having at least 60% homology therewith;
xlix) the amino acid sequence of nitroreductase ‘NfsB’, Escherichia coli (strain K12) (SEQ ID NO:81) or an amino acid sequence having at least 60% homology therewith;
1) the amino acid sequence of vanadium chloroperoxidase ‘CPO’ or ‘CiVHPO’, Curvularia inaequalis (SEQ ID NO:82) or an amino acid sequence having at least 60% homology therewith;
li) the amino acid sequence of aromatic unspecified peroxygenase ‘APO1’ or ‘AaeUPO’, Agrocybe aegerita (Black poplar mushroom) (Agaricus aegerita) (SEQ ID NO:83) or an amino acid sequence having at least 60% homology therewith;
   or a functional fragment, derivative or variant thereof.

Still more preferably, the second polypeptide is selected from or comprises
i) the amino acid sequence of Chromate Reductase, ‘TsOYE’ from Thermus scotoductus (SEQ ID NO:31) or an amino acid sequence having at least 60% homology therewith;
ii) the amino acid sequence of NADPH Dehydrogenase 2, ‘OYE-2’, Saccharomyces cerevisiae strain ATCC 204508 S288c (SEQ ID NO:33) or an amino acid sequence having at least 60% homology therewith;
iii) the amino acid sequence of Xenobiotic Reductase A, ‘XenA’, Pseudomonas putida (SEQ ID NO:35) or an amino acid sequence having at least 60% homology therewith;
iv) the amino acid sequence of Tryptophan 5-Halogenase, ‘PyrH’, Streptomyces rugosporus (SEQ ID NO:40) or an amino acid sequence having at least 60% homology therewith;
v) the amino acid sequence of Thermophilic Tryptophan Halogenase, ‘Th-Hal’, Streptomyces violaceusnige (SEQ ID NO:43) or an amino acid sequence having at least 60% homology therewith;
vi) the amino acid sequence of Halogenase, ‘PltM’, Pseudomonas fluorescens (strain ATCC BAA-477 NRRL B-23932 Pf-5) (SEQ ID NO:48) or an amino acid sequence having at least 60% homology therewith;
vii) the amino acid sequence of Styrene Monooxygenase, ‘StyA’, Pseudomonas sp. (SEQ ID NO:53) or an amino acid sequence having at least 60% homology therewith;
viii) the amino acid sequence of Chlorophenol Monooxygenase, ‘HadA’, Ralstonia pickettii (Burkholderia pickettii) (SEQ ID NO:56) or an amino acid sequence having at least 60% homology therewith;
ix) the amino acid sequence of 2,5-Diketocamphane 1,2-Monooxygenase 1, ‘CamP’, Pseudomonas putida (Arthrobacter siderocapsulatus) (SEQ ID NO:66) or an amino acid sequence having at least 60% homology therewith;
x) the amino acid sequence of Alkanal monooxygenase, alpha and beta chain, ‘LuxAB’, Vibrio harveyi (Beneckea harveyi) (SEQ ID NOs:68 and 69) or an amino acid sequence having at least 60% homology therewith;
xi) the amino acid sequence of Isobutylamine N-hydroxylase, ‘IBAH’, Streptomyces viridifaciens (SEQ ID NO:74) or an amino acid sequence having at least 60% homology therewith;
xii) the amino acid sequence of NAD(P)H-Flavin Reductase, ‘Fre’, Escherichia coli (strain K12) (SEQ ID NO:79) or an amino acid sequence having at least 60% homology therewith;
   or a functional fragment, derivative or variant thereof.

Preferably, when the second polypeptide comprises or consists of one or more amino acid sequences having at least 60% homology with a specified sequence, each amino acid sequence independently has at least 70%, such as at least 80%, more preferably at least 90%, e.g. at least 95%, preferably at least 97%, such as at least 98%, preferably at least 99% homology with the specified sequence. More preferably, each amino acid sequence independently has at least 70%, such as at least 80%, more preferably at least 90%, e.g. at least 95%, preferably at least 97%, such as at least 98%, preferably at least 99% identity with the specified sequence. For avoidance of doubt, if the second polypeptide comprises two or more amino acid sequences, the percentage homology of each of the two or more sequences with respect to their respective specified sequences can be the same or different, preferably the same. Percentage homology and/or percentage identity are each preferably determined across the length of the specified sequence.

As explained above, the choice of the second polypeptide is an operational parameter which can be controlled in order to obtain the desired reaction product. The methods of the invention can thus be used to produce a variety of functional groups within molecules. For example, Olefins (alkenes) can be reduced to alkane groups, e.g. using ene reductases. Halogenated products (e.g. brominated, chlorinated or fluorinated products) can be obtained using halogenases. Nitro groups such as aromatic nitro groups can be reduced (e.g. to their corresponding amine or hydroxylamine groups) using nitroreductases. Oxygen insertion reactions can be used to produce e.g. alcohols and ethers, etc, using monooxygenases. The methods of the invention find utility particularly in the production of complex products such as in synthesis or derivatisation of natural products, and in pharmaceutical production. The stereochemistry of the reaction can typically be controlled by appropriate selection of the second polypeptide. The choice of appropriate second polypeptide and the characterisation of the products obtained from the methods of the invention is well within the capacity of those skilled in the art. For example, products can be characterised by chemical analytical techniques such as IR spectroscopy, NMR, GC (including chiral-phase GC), polarimetry etc.

### Polypeptides Used in the Invention

Methods for expression of proteins in cellular (e.g. microbial) expression systems are well known and routine to those skilled in the art. For example, the first polypeptide and the second polypeptide (if present) can be independently isolated from their host organisms using routine purification methods. For example, host cells can be grown in a suitable medium. Lysing of cells allows internal components of the cells to be accessed. Membrane proteins can be solubilised with detergents such as Triton X (e.g. Triton X-114, (1,1,3,3-Tetramethylbutyl)phenyl-polyethylene glycol, available from Sigma Aldrich). Soluble or solubilized proteins can be isolated and purified using standard chromatographic techniques such as size exclusion chromatography, ion exchange chromatography and hydrophobic interaction chromatography. Alternatively, the first polypeptide and the second polypeptide (if present) can be independently encoded in one or more nucleotide vector and subsequently expressed in an appropriate host cell (e.g. a microbial cell, such as E. coli). Purification tags such as a HIS (hexa-histidine) tag can be encoded (typically at the C- or N-terminal of the relevant polypeptide) and can be used to isolate the tagged protein using affinity chromatography for example using nickel- or cobalt-NTA chromatography. If desired, protease recognition sequences can be incorporated between the first and/or second polypeptide and the affinity purification tag to allow the tag to be removed post expression. Such techniques are routine to those skilled in the art and are described in, for example, Sambrook et al, “Molecular Cloning: A Laboratory Manual”, Cold Spring Harbor Laboratory Press.

As described herein the first polypeptide and/or the second polypeptide (if present) may be a functional fragment, derivative or variant of an enzyme or amino acid sequence. As those skilled in the art will appreciate, fragments of amino acid sequences include deletion variants of such sequences wherein one or more, such as at least 1, 2, 5, 10, 20, 50 or 100 amino acids are deleted. Deletion may occur at the C-terminus or N-terminus of the native sequence or within the native sequence. Typically, deletion of one or more amino acids does not influence the residues immediately surrounding the active site of an enzyme. Derivatives of amino acid sequences include post-translationally modified sequences including sequences which are modified in vivo or ex vivo. Many different protein modifications are known to those skilled in the art and include modifications to introduce new functionalities to amino acid residues, modifications to protect reactive amino acid residues or modifications to couple amino acid residues to chemical moieties such as reactive functional groups on linkers or substrates (surfaces) for attachment to such amino acid residues.

Derivatives of amino acid sequences include addition variants of such sequences wherein one or more, such as at least 1, 2, 5, 10, 20, 50 or 100 amino acids are added or introduced into the native sequence. Addition may occur at the C-terminus or N-terminus of the native sequence or within the native sequence. Typically, addition of one or more amino acids does not influence the residues immediately surrounding the active site of an enzyme.

Variants of amino acid sequences include sequences wherein one or more amino acid such as at least 1, 2, 5, 10, 20, 50 or 100 amino acid residues in the native sequence are exchanged for one or more non-native residues. Such variants can thus comprise point mutations or can be more profound e.g. native chemical ligation can be used to splice non-native amino acid sequences into partial native sequences to produce variants of native enzymes. Variants of amino acid sequences include sequences carrying naturally occurring amino acids and/or unnatural amino acids. Variants, derivatives and functional fragments of the aforementioned amino acid sequences retain at least some of the activity/functionality of the native/wild-type sequence. Preferably, variants, derivatives and functional fragments of the aforementioned sequences have increased/improved activity/functionality when compared to the native/wild-type sequence.

Variants of an enzyme, such as the first polypeptide or second polypeptide as described herein, may preferably be modified to have an increased catalytic activity for their respective substrates. Preferably, the catalytic activity is increased at least 2 times, such as at least 5 times, e.g. at least 10 times, such as at least 100 times, preferably at least 1000 times. Catalytic activity can be determined in any suitable method. For example, the catalytic activity can be associated with the Michaelis constant K_{M} (with increased activity being typically associated with decreased K_{M} values) or with the catalytic rate constant, k_{cat} (with increased activity being typically associated with increased k_{cat} values).

Measuring K_{M} and k_{cat} is routine to those skilled in the art. For example, the K_{M} of a polypeptide for a substrate can be determined spectrophotometrically, e.g. by measuring absorption at 578 nm under anaerobic conditions at 30° C. in 50 mM Tris-HCl buffer, pH 8.0, containing 1 mM substrate, 5 mM benzyl viologen (oxidized; F=8.9 mM⁻¹ cm⁻¹), 90 μM dithionite, and 10 to 30 pmol of enzyme. Examples of solution assays in which the absorbance of oxidised and reduced flavins are determined are described in the examples.

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10). Software for performing BLAST analyses is publicly available through the National Center for Biotechnology Information (http://www.ncbi.nlm.nih.gov/).

Similarity can be measured using pairwise identity or by applying a scoring matrix such as BLOSUM62 and converting to an equivalent identity. Since they represent functional rather than evolved changes, deliberately mutated positions would be masked when determining homology. Similarity may be determined more sensitively by the application of position-specific scoring matrices using, for example, PSIBLAST on a comprehensive database of protein sequences. A different scoring matrix could be used that reflect amino acid chemico-physical properties rather than frequency of substitution over evolutionary time scales (e.g. charge). Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A below. Where amino acids have similar polarity, this can also be determined by reference to the hydropathy scale for amino acid side chains in Table B.

TABLE A
Chemical properties of amino acids

| | | | |
|---|---|---|---|
| Ala | aliphatic, hydrophobic, | Met | hydrophobic, neutral |
| | neutral | | |
| Cys | polar, hydrophobic, | Asn | polar, hydrophilic, |
| | neutral | | neutral |
| Asp | polar, hydrophilic, | Pro | hydrophobic, neutral |
| | charged (−) | | |
| Glu | polar, hydrophilic, | Gln | polar, hydrophilic, |
| | charged (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, | Thr | polar, hydrophilic, |
| | hydrophilic, charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE B
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

Preferably, sequence homology can be assessed in terms of sequence identity. Any of a variety of sequence alignment methods can be used to determine percent identity, including, without limitation, global methods, local methods and hybrid methods, such as, e.g., segment approach methods. Protocols to determine percent identity are routine procedures within the scope of those skilled in the art. Global methods align sequences from the beginning to the end of the molecule and determine the best alignment by adding up scores of individual residue pairs and by imposing gap penalties. Preferred methods include CLUSTAL W (Thompson et al., Nucleic Acids Research, 22(22) 4673-4680 (1994)) and iterative refinement (Gotoh, J. Mol. Biol. 264(4) 823-838 (1996)). Local methods align sequences by identifying one or more conserved motifs shared by all of the input sequences. Preferred methods include Match-box, (Depiereux and Feytmans, CABIOS 8(5) 501-509 (1992)); Gibbs sampling, (Lawrence et al., Science 262(5131) 208-214 (1993)); and Align-M (Van Walle et al., Bioinformatics, 20(9) 1428-1435 (2004)). Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

### Alignment Scores for Determining Sequence Identity

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Percent identity is then calculated as:

100×(T/L)

where
T=Total number of identical matches
L=Length of the longer sequence plus the number of gaps introduced into the longer sequence in order to align the two sequences

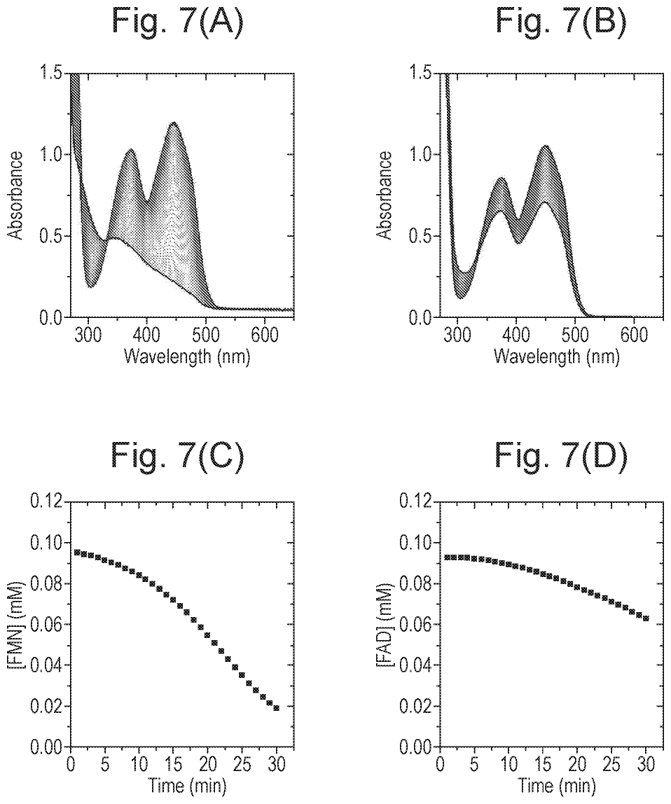

The first polypeptide and the second polypeptide when present may be distinct. However, in other embodiments, the first polypeptide is attached to the second polypeptide. This is shown schematically in FIG. 5. Any suitable attachment means may be used.

For example, the first polypeptide and second polypeptide may be attached together by chemical means. For example, cross-linking reagents can be used to attach the first polypeptide to the second polypeptide. Any suitable cross-linking reagent can be used. Suitable cross-linking reagents are known in the art. Functional groups that can be targeted with cross-linking agents include primary amines, carboxyls, sulfhydryls, carbohydrates and carboxylic acids. The cross-linking agent may be homobifunctional or heterobifunctional. Cross-linking reagents include bis(2-[succinimidooxycarbonyloxy]ethyl) sulfone, 1,4-di-(3′-[2′pyridyldithio]-propionamido) butane, disuccinimidyl suberate, disuccinimidyl tartrate, sulfodisuccinimidyl tartrate, dithiobis(succinimidyl propionate) (Lomant's reagent), 3,3′-dithiobis(sulfosuccinimidyl propionate), ethylene glycol bis(succinimidyl succinate), m-maleimidobenzoyl-N-hydroxysuccinimide ester, m-maleimidobenzoyl-N-hydroxysulfosuccinimide ester, N-γ-maleimidobutyryloxysuccinimide ester, N-γ-maleimidobutyryloxysulfosuccinimide ester, N-(ε-maleimidocaproic acid) hydrazide, N-(ε-maleimidocaproyloxy) succinimide ester, N-(ε-maleimidocaproyloxy) sulfo succinimide ester, N-(p-maleimidophenyl) isocyanate, N-succinimidyl(4-iodoacetyl)aminobenzoate, succinimidyl 4-(N-maleimidomethyl) cyclohexane1-carboxylate, succinimidyl 4-(p-maleimidophenyl) butyrate, N-sulfosuccinimidyl(4-iodoacetyl)aminobenzoate, sulfosuccinimidyl 4-(N-maleimidomethyl) cyclohexane-1-carboxylate, sulfosuccinimidyl 4-(N-maleimidomethyl) cyclohexane-1-carboxylate, sulfo succinimidyl 4-(p-maleimidophenyl) butyrate, 1-ethyl-3-(3-dimethylaminopropyl) carbodiimide hydrochloride, maleimide PEG N-hydroxysuccinimide ester, ρ-azidobenzoyl hydrazide, N-5-azido-2-nitrobenzyloxysuccinimide, ρ-azidophenyl glyoxal monohydrate, N-(4-[p-azidosalicylamido]butyl)-3′-(2′-pyridyldithio) propionamide, bis(p-[4-azidosalicylamido]-ethyl) disulphide, N-hydroxysuccinimideyl-4-azidosalicyclic acid, N-hydroxysulfosuccinimidyl-4-azidobenzoate, sulfosuccinimidyl 2-(7-azido-4-methylcoumarin-3-acetamide)ethyl-1,3-dithiopropionate, sulfosuccinimidyl 2-(m-azido-o-nitrobenzamido)-ethyl-1,3′-propionate, sulfosuccinimidyl 6-(4′-azido-2′-nitrophenylamino)hexanoate, sulfosuccinimidyl (4-azidophenyl dithio)propionate, sulfosuccinimidyl-2-(ρ-azidosalicylamido)ethyl-1,3-dithiopropionate, and the like. Glutaraldehyde may be used. The first and second polypeptide can be attached together by ECD/NHS coupling.

Alternatively, the first and second polypeptide may be genetically fused together. The first polypeptide and second polypeptide may for example by attached by a genetically encoded linker e.g. comprising (SG)n units (wherein n is typically from about 4 to about 50). A construct between the first and second polypeptide may be produced by native chemical ligation. Methods of cloning and expressing fusion polypeptides are well known to those skilled in the art and are described in, for example, Sambrook et al, “Molecular Cloning: A Laboratory Manual”, Cold Spring Harbor Laboratory Press.

A construct comprising the first and second polypeptide attached together, for example, as described above, is provided as a further aspect of the invention.

### Reaction Conditions

In the invention, the first polypeptide and optionally the second polypeptide if present may be in solution. The concentration of the first and/or second polypeptide in solution is an operational parameter that can be controlled by the user to obtain required outputs. Typical concentrations are in the range of from about 1 to about 1000 μg of protein per mL of solution, such as from about 10 to about 100 μg/mL, e.g. from about 25 to about 75 μg/mL. The first polypeptide may be provided in a first solution and the second polypeptide provided in a second solution, and the first and second solutions may be mixed. Alternatively the first and second polypeptide may be co-formulated in a single solution.

Alternatively, the first polypeptide and optionally the second polypeptide if present may be immobilised on a support e.g. a solid support. The first polypeptide may be immobilised on a different solid support to the second polypeptide if present. The first polypeptide and second polypeptide if present may be immobilised on separate supports wherein the supports are the same type of support or are different types of support. The first polypeptide and second polypeptide may be immobilised on the same support. The first polypeptide and the second polypeptide may be co-immobilised on the support. The first polypeptide and the second polypeptide may be mixed and the mixture of the first and second polypeptides may be immobilised on the support. Alternatively the first polypeptide may be immobilised on the support and then the second polypeptide may be immobilised on the support. Still alternatively, the second polypeptide may be immobilised on the support and then the first polypeptide may be immobilised on the support. The first polypeptide may be provided in solution and the second polypeptide may be immobilised on a support. Alternatively the first polypeptide may be immobilised on a support and the second polypeptide may be provided in solution.

As used herein, the term “immobilized” embraces adsorption, entrapment and/or cross-linkage between the support and the polypeptide. Adsorption embraces non-covalent interactions including electrostatic interactions, hydrophobic interactions, and the like. A charged adsorption enhancer such as polymyxin B sulphate can be used to enhance adsorption. Entrapment embraces containment of the polypeptide onto the surface of the support, e.g. within a polymeric film or in a hydrogel. Cross-linkage embraces covalent attachment, either directly between the polypeptide (e.g. via amide coupling, such as via EDC/NHS and/or other coupling agents routine to those skilled in the art) or using one or more covalent cross-linkers such as thiol-terminated linkers or crosslinking reagents. Immobilization means comprising or consisting of adsorption are preferred. Combination of some or all of the above mentioned immobilization means may be used.

Typically, the or each support independently comprises a material comprising carbon, silica, a metal or metal alloy, a metal oxide (include mixed metal oxides, e.g. titanium, aluminium and zirconium oxides), a metal hydroxide (including layered double hydroxides), a metal chalcogenide, or a polymer (e.g. polyaniline, polyamide, polystyrene, etc); or mixtures thereof. As those skilled in the art will appreciated, suitable support materials can include mixtures of materials described herein. Any suitable support material can be used. Resins and glasses can be used.

Sometimes, the or each support material comprises a carbon material. Suitable carbon materials include graphite, carbon nanotube(s), carbon black, activated carbon, carbon nanopowder, vitreous carbon, carbon fibre(s), carbon cloth, carbon felt, carbon paper, graphene, and the like. Sometimes the or each support material comprises a mineral such as bentonite, halloysite, kaolinite, montmorillonite, sepiolitem and hydroxyapatite.

Sometimes the or each support material comprises a biological material such as collagen, cellulose, keratin, carrageenan, chitin, chitosan, alginate and agarose.

Suitable materials may be in the form of a particle. Typical particle sizes are from about 1 nm to about 100 μm, such as from about 10 nm to about 10 μm e.g. from about 100 nm to about 1 μm. Methods of determining particle size are routine in the art and include, for example, dynamic light scattering. Support materials of appropriate size are readily available from commercial suppliers. For example, carbon black particles such as “Black Pearls 2000” particles are available from Cabot corp (Boston, Mass., USA).

A benefit which arises from the support of the first and/or second polypeptide is that the polypeptides can be easily removed from the reaction mixture. For example, the support(s) can be removed by sedimentation, filtration, centrifugation, or the like. Many such methods are known to those skilled in the art, e.g. filtration can be achieved using a simple filter paper to remove solid components from a liquid composition; or a mixed solid/liquid composition can be allowed to settle and the liquid then decanted from the settled solids.

The first polypeptide and second polypeptide if present may be present in a biological cell. This is an example of whole cell catalysis. The first polypeptide may be present in a different biological cell to the second polypeptide if present. The first polypeptide and second polypeptide if present may be present in biological cells wherein the cells are the same type of support or are different types of cell. The first polypeptide and second polypeptide may be present in the same biological cell.

The first polypeptide may be an exogenous polypeptide which is expressed non-natively by the cell. Alternatively the first polypeptide may be a native polypeptide which is natively expressed by the cell. The first polypeptide may be a native polypeptide in the cell and expressed under native conditions. The first polypeptide may be a native polypeptide in the cell, but expressed under non-native conditions, e.g. the first polypeptide may be overexpressed in the cell. The second polypeptide if present may be an exogenous polypeptide which is expressed non-natively by the cell. Alternatively the second polypeptide may be a native polypeptide which is natively expressed by the cell. The second polypeptide may be a native polypeptide in the cell and expressed under native conditions. The second polypeptide may be a native polypeptide in the cell, but expressed under non-native conditions, e.g. the second polypeptide may be overexpressed in the cell. Methods of cloning and expressing polypeptides in cells are well known to those skilled in the art and are described in, for example, Sambrook et al, “Molecular Cloning: A Laboratory Manual”, Cold Spring Harbor Laboratory Press. When the first and/or second polypeptide is present in a cell, the cell may be supported on a support as described herein.

When the first and/or second polypeptide is present in a cell, any suitable cell may be used. Typically, the cell is a bacterial or archaeal cell. Usually, the cell is a bacterial cell. Suitable bacterial cells include Escherichia cells, Ralstonia (also referred to as Cupriavidus) cells and Pseudomonas cells. For example, the bacterial cell may be an E. coli cell, a Pseudomonas aeruginosa cell or a Ralstonia eutropha (also known as Cupriavidus necator) cell.

In the methods of the invention, the cofactor is preferably initially added to or present in an aqueous solution at a concentration of 1 μM to 1 M, such as from 5 μM to 800 mM, e.g. from 10 μM to 600 mM such as from 25 μM to 400 mM e.g. from 50 μM to 200 mM such as from 100 μM to about 100 mM e.g. from about 250 μM to about 10 mM such as from about 500 μM to about 1 mM.

As explained above, the methods of the invention are typically conducted under a gas atmosphere; i.e. in the presence of gas (for example in the headspace of a reactor). Preferably, the gas atmosphere comprises hydrogen or an isotope thereof and optionally an inert gas. O₂ or an isotope thereof may be present. Preferred inert gases include nitrogen, argon, helium, neon, krypton, xenon, radon and sulfur hexafluoride (SF₆) and mixtures thereof, more preferably nitrogen and/or argon, most preferably nitrogen. When the gas atmosphere comprises a mixture of hydrogen and an inert gas and/or O₂, the hydrogen is preferably present at a concentration of 1-100%, with the remaining gas comprising an inert gas as defined herein and/or O₂. Preferred gas atmospheres include from 80-100% H₂ with the remaining gas comprising one or more inert gases; and from 0-20% H₂ with the remaining gas comprising one or more inert gases and/or O₂ (such as from 14% H₂ in air). The gas atmosphere may optionally also include non-inert gases such as ammonia, carbon dioxide and hydrogen sulphide. Preferably, however, the gas atmosphere is free of ammonia, carbon dioxide and hydrogen sulphide. The methods of the invention may be conducted at any suitable pressure: selecting an appropriate pressure is an operational parameter of the methods of the invention which can be controlled by the operator. Sometimes, the methods of the invention are conducted at ambient pressure (e.g. about 1 bar). Sometimes, the methods of the invention are conducted at reduced pressure (e.g. less than 1 bar) or at elevated pressure (e.g. greater than 1 bar). For example, increasing the operating pressure can increase hydrogen solubility in the reaction medium. Preferably, the methods of the invention are carried out at a pressure of from about 0.1 bar to about 20 bar, such as from about 1 bar to about 10 bar, e.g. from about 2 bar to about 8 bar such as from about 4 bar to about 6 bar, e.g. about 5 bar.

Typically, the methods of the invention are carried out under aerobic conditions. As used herein, “aerobic conditions” refers to the gas atmosphere not being strictly anaerobic, e.g. comprising at least trace O₂. Suitable O₂ levels are typically greater than 100 ppm, e.g. greater than 1000 ppm (0.1%), such as greater than 1% O₂, for example greater than 2% 02. Usually, 02 levels do not exceed the O₂ levels in atmospheric air, i.e. 21% O₂, however greater O₂ levels are not excluded.

The methods of the invention are typically conducted in an aqueous composition which may optionally comprise e.g. buffer salts. For some applications buffers are not required and the methods of the invention can be conducted without any buffering agents. Preferred buffer salts which can be used in the methods of the invention include Tris; phosphate; citric acid/Na₂HPO₄; citric acid/sodium citrate; sodium acetate/acetic acid; Na₂HPO₄/NaH₂PO₄; imidazole (glyoxaline)/HCl; sodium carbonate/sodium bicarbonate; ammonium carbonate/ammonium bicarbonate; MES; Bis-Tris; ADA; aces; PIPES; MOPSO; Bis-Tris Propane; BES; MOPS; TES; HEPES; DIPSO; MOBS; TAPSO; Trizma; HEPPSO; POPSO; TEA; EPPS; Tricine; Gly-Gly; Bicine; HEPBS; TAPS; AMPD; TABS; AMPSO; CHES; CAPSO; AMP; CAPS and CABS. Selection of appropriate buffers for a desired pH is routine to those skilled in the art, and guidance is available at e.g. http://www.sigmaaldrich.com/life-science/core-bioreagents/biological-buffers/learning-center/buffer-reference-center.html. Buffer salts are preferably used at concentrations of from 1 mM to 1 M, preferably from 10 mM to 100 mM such as about 50 mM in solution. Most preferred buffers for use in methods of the invention include 50 mM phosphate, pH 8.0.

The methods of the invention are typically conducted in an aqueous composition. However, non-aqueous components can optionally be used instead or as well as water in the compositions used in the methods of the invention. For example, one or more organic solvents (e.g. alcohols, DMSO, acetonitrile, etc) or one or more ionic liquids may be used or included in the compositions.

The methods of the invention are typically carried out at a temperature of from about 20° C. to about 80°, such as from about 25° C. to about 60° C., e.g. from about 30° C. to about 50° C.

The methods of the invention may be performed in an apparatus as provided herein. The apparatus typically comprises a reaction vessel. The reaction vessel typically comprises one or more inlets for molecular hydrogen gas or hydrogen-containing liquids (e.g. hydrogen saturated liquids such as buffer solutions as described herein); and/or one or more inlet for reagents; and/or one or more outlets for product. Further equipment such a pressure controls, temperature controls, mixing apparatus, flow controls, etc may be incorporated. The apparatus may be comprised as a part of an apparatus for converting initial reagents into final products and thus be configured to perform an intermediate reaction step. The apparatus may be controlled by equipment such as a computer controller. The apparatus may comprise means for detecting cofactor turnover, reagent utilisation and/or product production, e.g. spectrophotometric means. The apparatus may be configured to be operated in flow mode (i.e. continuous mode) or in batch mode.

Accordingly, the methods provided herein may be performed in a flow setup, e.g. in a flow reaction cell. The methods provided herein may alternatively be performed in a batch setup e.g. in a batch reaction cell.

### Further Methods

The invention also provides a method of reducing an oxidised flavin cofactor, comprising:
contacting the oxidised flavin cofactor and molecular hydrogen (¹H₂) or an isotope thereof with a first polypeptide which is a hydrogenase enzyme or a functional fragment or derivative thereof under conditions such that the oxidised flavin cofactor is reduced to form a reduced flavin cofactor;
wherein the first polypeptide does not comprise a native flavin active site for NAD(P)⁺ reduction.

Preferably, such methods further comprise the re-oxidation of the reduced flavin cofactor to regenerate the oxidised flavin cofactor. Preferably, such method steps are repeated multiple times thereby recycling the cofactor.

In such methods, the oxidised flavin is typically as defined herein. The first polypeptide is typically as defined herein. In some embodiments the first polypeptide is in solution. In other embodiments the first polypeptide is immobilised on a solid support. Sometimes, the first polypeptide is comprised in a biological cell as defined here. Often, the reaction conditions are as set out in more detail herein. Other features of this aspect of the invention are typically as set out herein.

### System

The invention also provides a system for performing a method of the invention.

The invention thus provides a system for reducing an oxidised flavin cofactor, comprising:
a first polypeptide which is a hydrogenase enzyme or a functional fragment or derivative thereof,
the oxidised flavin cofactor; and
molecular hydrogen (¹H₂) or an isotope thereof;
   wherein the first polypeptide does not comprise a native flavin active site for NAD(P)⁺ reduction.

The invention also provides a system for producing a reaction product, comprising:
a first polypeptide which is a hydrogenase enzyme or a functional fragment or derivative thereof,
a flavin cofactor;
a second polypeptide which is an oxidoreductase or a functional fragment or derivative thereof,
molecular hydrogen (¹H₂) or an isotope thereof, and
a reactant for conversion to said reaction product.

In the systems of the invention, the flavin cofactor is typically as defined herein. The first polypeptide and second polypeptide if present are typically each as defined herein. The first polypeptide and second polypeptide is present are typically independently in solution, or are immobilised on a solid support. The first polypeptide and second polypeptide if present may be comprised in a biological cell. The first polypeptide and second polypeptide if present may be attached to each other. The system may be configured to be operated as described for the methods provided herein.

Typically, in the systems of the invention, one or both of the enzymes is in solution or is supported on a support as described herein. Typically, the system may further comprise means for controlling the gas atmosphere in the system, such as a gas flow system. The system is often configured as a flow cell containing reagents as described herein. The system (e.g. a flow cell) may comprise features of the provided apparatus described herein, such as one or more inlets for molecular hydrogen gas or hydrogen-containing liquids and/or one or more inlet for reagents; and/or one or more outlets for product; and/or one or more pressure controls, temperature controls, mixing apparatus, flow controls, etc

The following Examples illustrate the invention. They do not, however, limit the invention in any way. In this regard, it is important to understand that the particular assays used in the Examples section are designed only to provide an indication of the efficacy of the method of the invention. There are many assays available to determine reaction efficiency, and a negative result in any one particular assay is therefore not determinative.

### EXAMPLES

### Example 1

This example demonstrates a new activity for the [NiFe] uptake hydrogenase 1 of Escherichia coli (Hyd1) in accordance with the invention. Direct reduction of biological flavin cofactors FMN and FAD is achieved using H₂ as a simple, completely atom-economical reductant. The robust nature of Hyd1 is exploited for flavin reduction across a broad range of temperatures (25-70° C.) and extended reaction times. The utility of this system as a simple, easy to implement FMNH₂ regenerating system is then demonstrated by supplying reduced flavin to an Old Yellow Enzyme for asymmetric alkene reductions with up to 100% conversion. High Hyd1 turnover frequencies (up to 20.4 min⁻¹) and total turnover numbers (>20,000) during flavin recycling demonstrate the efficacy of this biocatalytic system.

As the need to make chemical manufacturing more sustainable becomes urgent, academic and industrial fields increasingly turn to biotechnology.^{[1]} Enzymes provide many advantages over other catalysts: they are renewable, biodegradable, nonhazardous, and provide high selectivity. The once-limited scope of known enzyme reactions has rapidly expanded, aided by enzyme engineering and ongoing discovery and characterisation of new enzymatic functions.^{[2,3]}

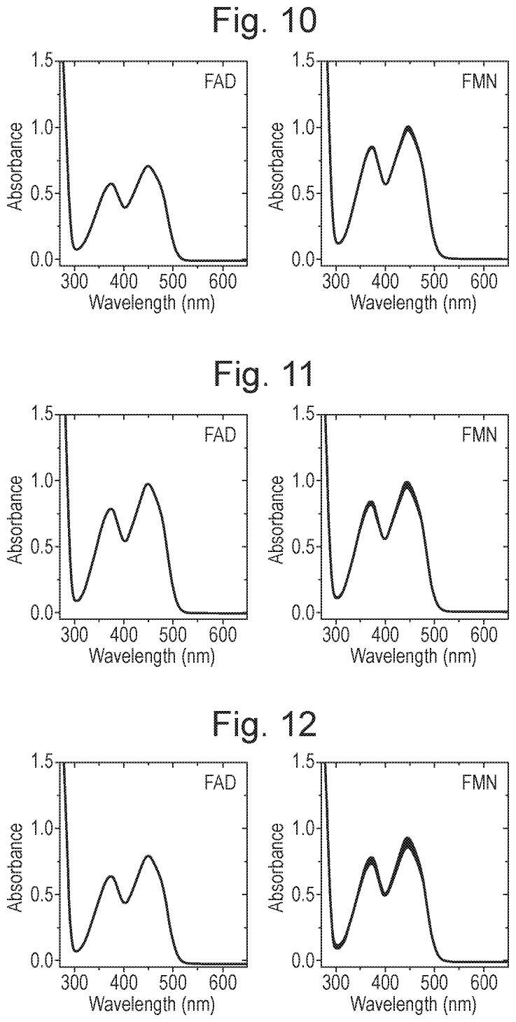

One class of synthetically useful enzymes are flavoenzymes, which contain or rely upon biological flavin cofactors (e.g. FMN, FAD; see below). For biotechnology, important flavoenzymes are halogenases (chlorination, bromination, iodination),^{[4]} ene-reductases (activated alkene reduction),^{[5]} and flavoprotein monooxygenases (epoxidations, hydroxylations, Baeyer-Villiger oxidation).^{[6]} Potential applications of these enzymes include natural product and pharmaceutical synthesis,^{[7]} biodegradation of environmental pollutants,^{[8]} and non-native light-driven reactions (FIG. 9).^{[9]} These reactions require one equivalent of the reduced cofactors FMNH₂ or FADH₂. To lower cost and waste, a catalytic quantity of more stable oxidised FMN/FAD is supplied, and is reduced in situ by means of photochemistry, electrochemistry, metal-catalysis or biocatalysis (FIG. 9).^{[4,5,10]}

In general, biocatalyzed cofactor recycling is the most straightforward option for coupling with flavoenzyme reactions because the alternative catalysts can face biocompatibility challenges (e.g. mutual inactivation, mismatched ideal solvent, pH or temperature).^{[4,11]} A common, yet cumbersome, strategy is to recycle the reduced flavin using an NAD(P)H-dependent reductase which produces FMNH₂ or FADH₂ at the expense of NAD(P)H^{[12]} or oxidised nicotinamide cofactor analogues.^{[13]} A catalytic quantity of the reduced nicotinamide cofactors must in turn be regenerated due to their high cost. This is typically achieved via glucose dehydrogenase-catalysed oxidation of glucose, which elevates cost, waste and downstream processing.^{[14]} The complexity of the currently-available recycling systems for reduced flavins may explain the under-utilisation of flavoenzymes in biotechnology, despite the important reactions they catalyse.^{[15]}

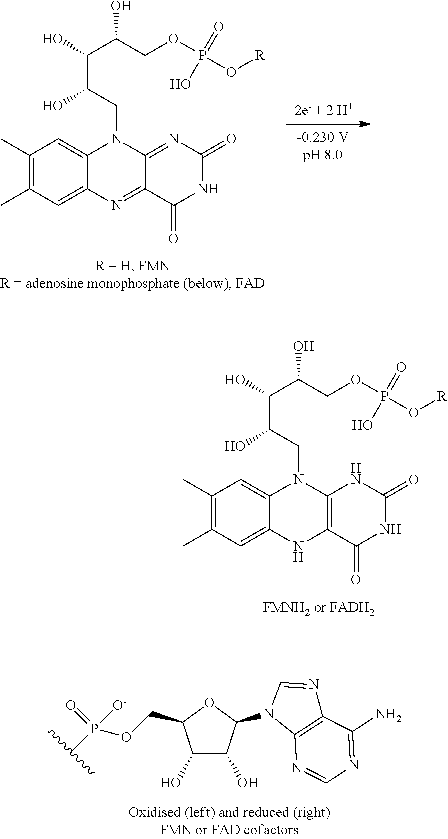

Alternatively, H₂ has previously been demonstrated for cleaner enzymatic NADH cofactor recycling.^{[16,17]} The soluble hydrogenase from Cupriavidus necator (formerly Alcaligenes eutrophus or Ralstonia eutropha) couples the reduction of NADY to NADH using H₂. This enzyme contains a prosthetic flavin cofactor at the NAD⁺ binding site where electrons from H₂ oxidation accumulate to reduce NAD⁺.^{[17]} Reduction of various substrates by this enzyme under H₂ has also been investigated.^{[18]} However, this enzyme is produced in the native host and requires extended incubation (7-10 days) in energy-depleted growth conditions for significant expression levels.^{[19]} Additionally, it lacks stability at elevated temperatures,^{[20]} preventing broad applicability.

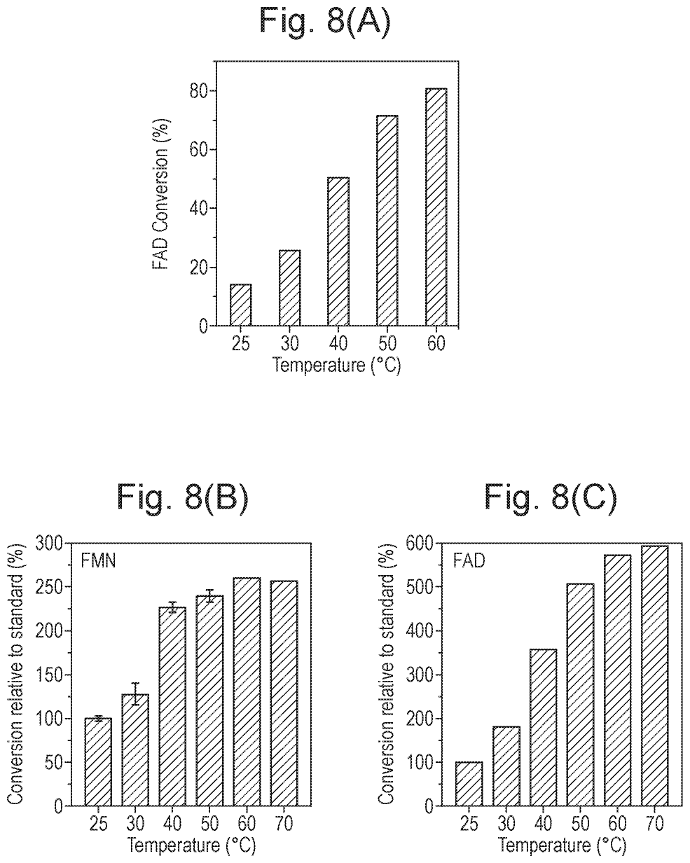

This inspired us to test whether a simple hydrogenase system (FIG. 6) could be suitable for H₂-driven flavin reduction. The thermodynamic potential for the H⁺/H₂ redox couple (−0.472 V, pH 8) is negative of the potential for flavin reduction (−0.231 V, pH 8)^{[21]}, making the reduction of flavin by H₂ thermodynamically favourable. We selected E. coli [NiFe]-hydrogenase (Hyd1), which is a good H₂ oxidiser^{[22,23]} and well-characterised in terms of X-ray crystal structures^{[24,25]} and spectroscopy.^{[23,26]} Hyd1 is natively expressed in E. coli and, unlike many hydrogenases,^{[27]} it is O₂-tolerant^{[23]} and active over a wide pH range.^{[28]} Like other uptake hydrogenases, the basic unit of Hyd1 is a 100 kDa heterodimer of the large subunit (L) housing the NiFe active site, and the small subunit (S) housing the iron-sulfur cluster electron transfer relay. Natively, Hyd1 is coupled to a cytochrome electron acceptor, and exists as a homodimer of SL units. The isolated enzyme we utilize here comprises predominantly dimeric SL units.

The H₂ oxidation activity of Hyd1 is typically measured using the artificial electron acceptor benzyl viologen in colourimetric assays.^{[28]} Electrons from H₂ oxidation at the [NiFe] active site (FIG. 6) are relayed through FeS clusters where, evidence suggests, benzyl viologen reduction occurs rather than directly at the NiFe active site.^{[29]} Herein, we demonstrate that both FMN and FAD can accept electrons from H₂ oxidation by Hyd1 to generate FMNH₂ and FADH₂, and we show that Hyd1 can be used as an effective FMNH₂ regeneration system to support asymmetric alkene reduction by an Old Yellow Enzyme (OYE)-type ene-reductase.

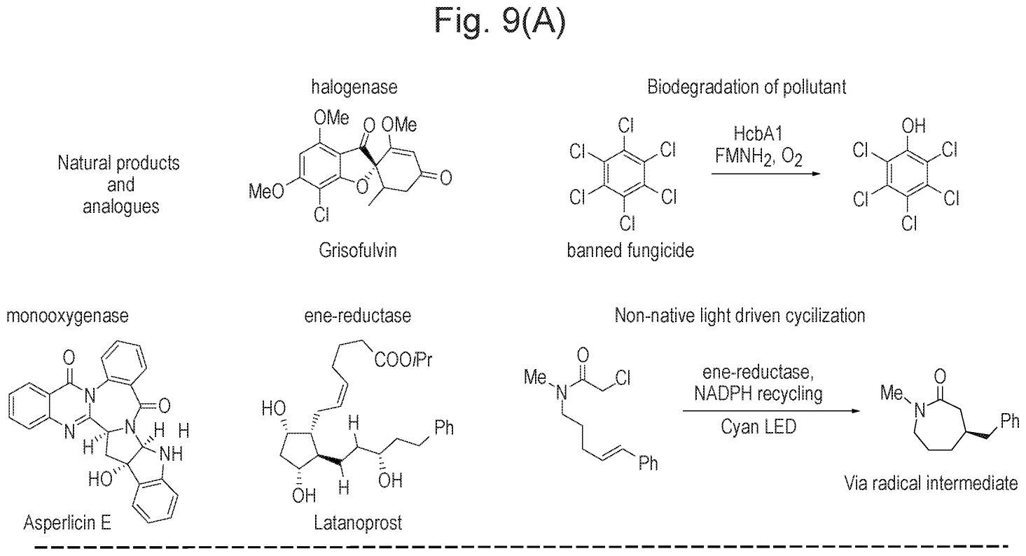

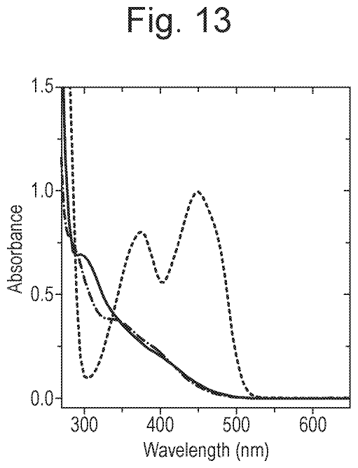

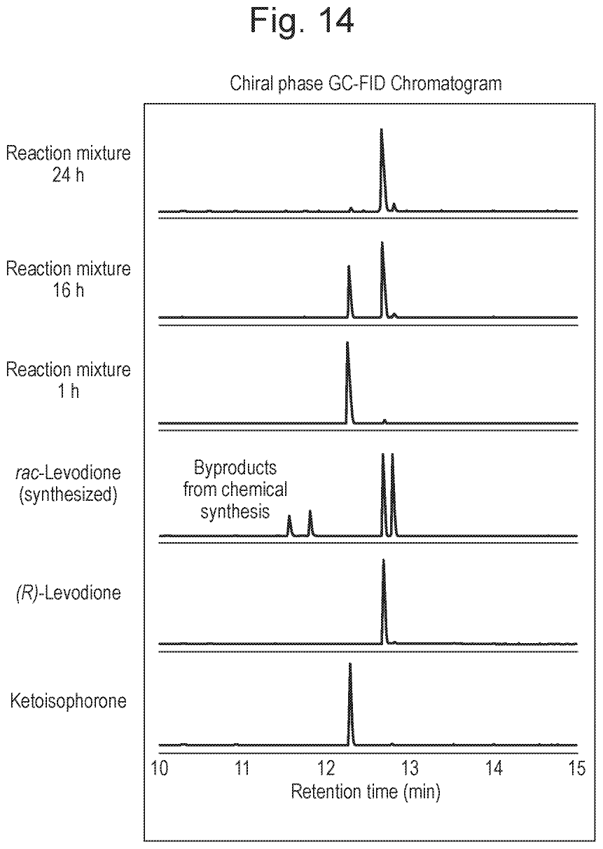

FIG. 7 shows the results of in situ UV-visible spectrophotometric assays to explore the possibility of FMN and FAD reduction by Hyd1 (38 mg, produced and isolated as described below, see Supporting Information) under H₂ (General Procedure A, Supporting Information). The flavin moiety of FMN gives λₘₐₓ at 445 nm and FAD at 450 nm, both of which disappear upon two-electron reduction (FIG. 7A-B; see FIG. 13 for spectra of fully reduced FMN)^{[30,31]}. The decrease in [oxidised flavin] over time was used to calculate initial enzyme activity (FIG. 7C-D). Control experiments indicated that omission of Hyd1 or H₂ led to negligible flavin reduction (FIGS. 10-11).

Upon addition of Hyd1, a lag phase was observed during FMN and FAD reduction, which is attributed to the well-characterised H₂-dependent activation phase for aerobically purified Hyd1.^{[23]} Later experiments (when indicated) used Hyd1 that was first activated under a H₂ atmosphere.^{[32]} The lag phase was followed by a decrease in absorbance consistent with FMNIIH₂/FADH₂ formation, and clear isosbestic points at 330 nm corroborated a lack of side products. Specific initial activities for FMN and FAD reduction (76 and 32 nmol min⁻¹ mg⁻¹ Hyd1, respectively) were determined during the linear reaction phase. The higher activity for FMN reduction compared with FAD cannot be attributed to thermodynamic driving force since both cofactors have similar reduction potentials,^{[21]} but could relate to the cofactors' ability to interact at the protein surface.

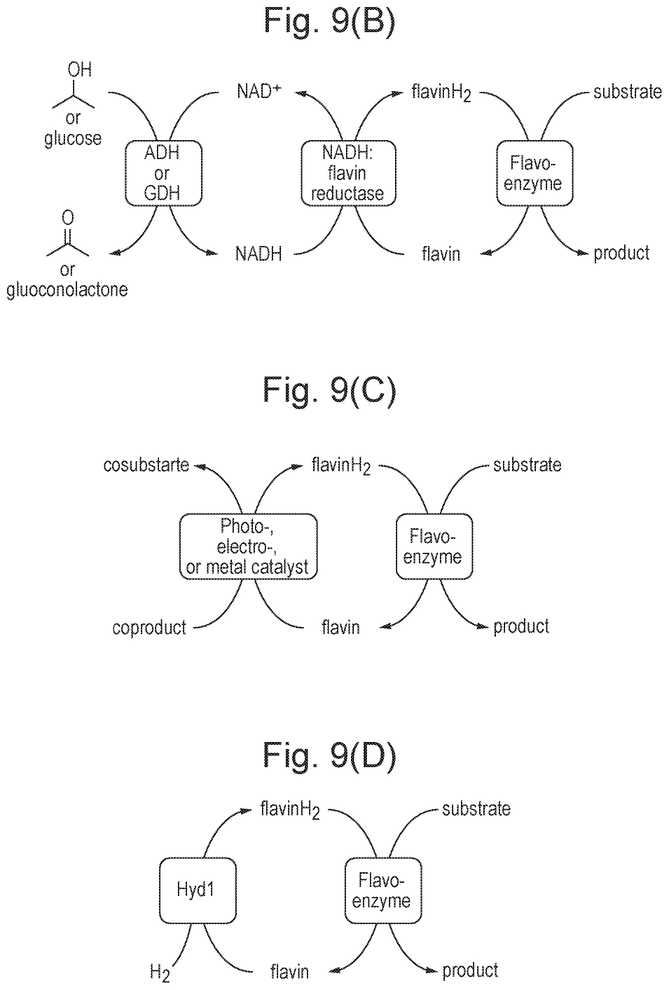

Hyd1 is known to be robust which inspired us to test H₂-driven flavin reduction activity at different temperatures (25-70° C., General Procedure A). Percentage conversion of FMN and FAD to the reduced forms after 30 min reaction time increased with temperature (FIG. 8), though FMN reduction was not enhanced past 60° C. This temperature and pH tolerance of Hyd1 is likely to open new doors to cofactor recycling for flavoenzymes with optimal activity at higher temperatures.

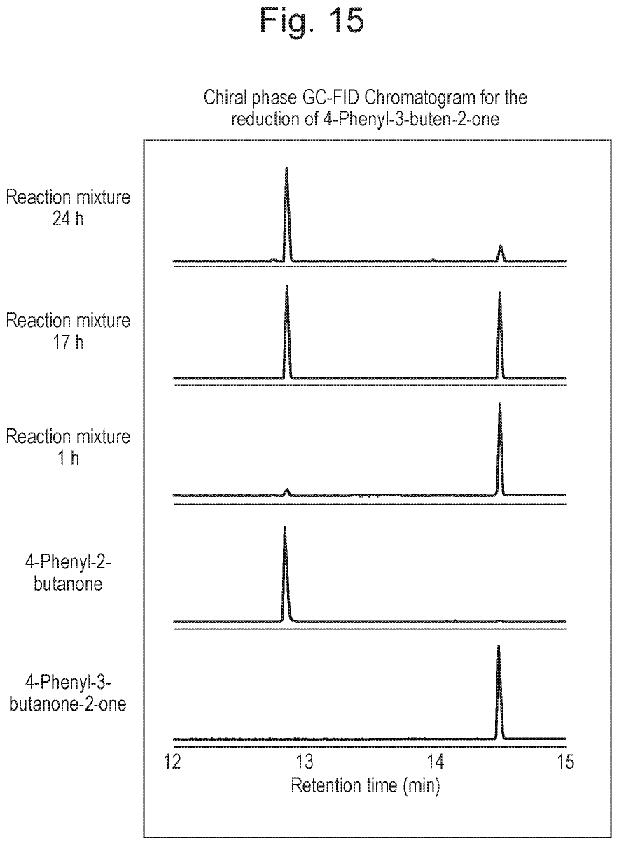

In order to demonstrate the utility of Hyd1 in biotechnologically-relevant flavin recycling, we coupled Hyd1-catalysed flavin reduction with the OYE-type ene-reductase from Thermus scotoductus, TsOYE,^{[33,34]} to catalyze enantioselective reduction of ketoisophorone (1) to (R)-levodione (2, see scheme below). Reactions were conducted according to General Procedure B (Supporting Information) and monitored using chiral-phase GC-FID after extraction of the mixture into ethyl acetate (see FIG. 14). Enantiomeric excess (ee) was always >99% at the first time point but decreased to 86-92% from slow racemisation under alkaline conditions. Control experiments confirmed that each component is required for conversion (Table 2).

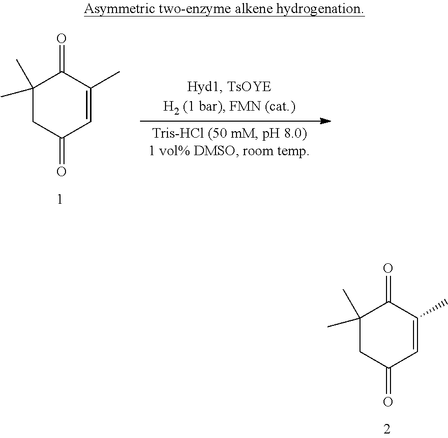

Quantitative conversion and the highest Hyd1 turnover frequency (TOF, 0.34 s⁻¹) was achieved with 0.5 mM FMN and 2 mM 1 (entry 1, Table 1). This TOF compares with or improves upon two-component NAD(P)H:flavin reductases, which unlike Hyd1 are rate-limited by the active site binding or release of two substrates.^{[15,35]}

When 0.1 mM FMN was used with varying [1] (entries 2-5), up to 97 FMN turnovers (TN) were achieved. This is comparable to the FMN TN reported for a formate-driven homogeneous Rh-catalyzed method for FMNH₂ recycling coupled to TsOYE.^{[33]} That system required careful balance between enzyme and Rh-catalyst loading to prevent non-enantioselective alkene reduction by FMNH₂ or [Cp*Rh(bpy)H]⁺, which was not an appreciable issue with our biocatalytic system (Table 2).

The highest Hyd1 total turnover number (TTN, 10,200) was achieved using 10 mM 1 (entry 4). This TTN is of an appropriate order of magnitude for industrial catalysis,^{[36]} but there remains room for further optimisation to that end. At 20 mM 1, Hyd1 TTN and conversion were boosted using 4 bar H₂, which also improved Hyd1 TOF from 0.9 s⁻¹ to 0.11 s⁻¹ (compare entries 5-6).

TABLE 1
H₂-Driven enzymatic alkene reduction under various conditions

| | [1] | [FMN] | Conv. to 2 | | Hyd1 | FMN |
|---|---|---|---|---|---|---|
| Entry | (mM) | (mM) | (%)^{a} | | TTN^{b} | TN^{b} |
| 1 | 2 | 0.5 | 100 | | 2,100 | 4 |
| 2 | 2 | 0.1 | 100 | | 2,100 | 20 |
| 3 | 5 | 0.1 | 95 | {100} | 5,200 | 50 |
| 4 | 10 | 0.1 | 62 | {97} | 10,200 | 97 |
| 5 | 20 | 0.1 | 24 | {37} | 7,800 | 74 |

| | | | | | |
|---|---|---|---|---|---|
| 6^{c} | 20 | 0.1 | {44} | 9,300 | 88 |

Reaction conditions: General procedure B using consistent catalyst loadings.
^{a}Chiral-phase GC conversion to 2 at 15 h {and 24 h}.
^{b}Hyd1 total turnover number (mol 2 per mol Hyd1) and FMN turnover number (mol 2 per mol flavin) were determined at the end of the reaction.
^{c}4 bar H₂ was used.

Like Hyd1, TsOYE has enhanced activity at elevated temperatures,^{[33]} therefore entry 4 was replicated at 35° C. (data not shown): Hyd1 TOF nearly doubled to 0.16 s⁻¹ and full conversion was achieved after 24 h, however GC-FID showed that some of 1 and 2 likely evaporated.

To test stability over time, Hyd1 (57 μg) was activated under H₂ at 22° C. over 58 h, then incubated in 0.08 mM FMN under H₂ (1 bar) in a sealed vessel for 62 h. Upon release of H₂, FMNH₂ partially oxidised under the N₂ atmosphere to 0.05 mM FMN (determined using UV-visible spectroscopy). The Hyd1 and FMN/FMNH₂ solution was placed back under H₂, and full reduction to FMNH₂ was noticed after 3.5 h (see Figure S5), which demonstrates appreciable Hyd1 stability over 125 h (>5 days).

The simplified, H₂-driven biocatalysed flavin recycling method coupled with TsOYE led to high Hyd1 TOF and TTN that correspond with commercial grade enzymes.^{[37]} Further modifications to Hyd1, which is tolerant of mutagenesis,^{[25,32]} might enhance the non-native activity. Additionally, process development is underway to improve industrially-relevant metrics such as cofactor TN. This proof of concept work shows that the robust Hyd1, tolerant to a range of conditions, is a promising catalyst to bring clean flavin recycling into biotechnology. This work thus demonstrates the efficacy and utility of the invention provided herein.

### Supporting Information

### Reagents

Buffer salts (Sigma Aldrich), FAD (disodium salt, >98%, Cayman Chemical Company), and FMN (monosodium salt dihydrate, Applichem Panreac) were all used as received. All aqueous solutions were prepared with deoxygenated MilliQ water (Millipore, 18 MΩcm). The hydrogenase (E. coli hydrogenase 1, Hyd1, molecular weight 100 kDa) was produced by homologous over-expression of the genes encoding the structural subunits of the enzyme and key maturases. After Hyd1 overexpression under anaerobic bacterial growth, the enzyme was isolated following published protocols^{[S2]} and stored at −80° C. as a 3.8 mg/mL solution in Tris-HCl buffer (20 mM Tris-HCl pH 7.2, 350 mM NaCl, 0.02% Triton X, 1 mM DTT). The Thermus scotoductus ene-reductase of the Old Yellow Enzyme family (TsOYE, molecular weight of homodimer 72.4 kDa)^{[S3,S4]} was isolated following published protocols.

### Analytical Tools

UV-visible spectra were recorded by a Cary 60 spectrophotometer with a cell holder (Agilent) and a Peltier accessory for temperature control using a quartz cuvette (path length 1 cm, cell volume 1 mL, Hellma). The indicated buffer was used to take a baseline scan. In some of the experiments, there was a uniform shift of the baseline across the entire spectral region (200-800 nm), which was corrected for during data processing. The concentration of FMN was directly calculated based on the absorbance at λ=445 nm (ε=12.50 mM⁻¹ cm⁻¹) and FAD based on the absorbance at λ=450 nm (ε=11.30 mM⁻¹ cm⁻¹).

The decrease in [oxidized flavin] over time was determined in order to calculate specific initial enzyme activity (not counting any lag phase).^{[S5]}

Chiral phase GC-FID to monitor alkene reductions was performed as follows:

Column: CP-Chirasil-Dex CB (Agilent), 25 m length, 0.25 mm diameter, 0.25 μm (film thickness), fitted with a guard of 10 m undeactivated fused silica of the same diameter

Carrier: He (CP grade), 170 kPa (constant pressure)

Inlet temperature: 200° C.

Injection conditions: Splitless with split flow 60 mL/min, splitless time 0.8 mins, purge 5 mL/min. Injection volume=0.5 μL.

Detection: FID (H₂=35 mL/min, air=350 mL/min, makeup N₂=40 mL/min, temp=200° C.)

Oven Heating Profile:

| Time (minutes) | Temperature |
|---|---|
| 0 → 5 | Hold at 70° C. |
| 5 → 30 | Ramp to 120° C. at 2° C./min |
| 30 → 36 | Ramp to 180° C. at 10° C./min |
| 36 → 45 | Hold at 180° C. for 5 minutes |

Compound Retention Times (Reduction of 1):

| Time (minutes) | Compound |
|---|---|
| 12.27 | Ketoisophorone (1) |
| 12.68 | (R)-Levodione (2) |
| 12.80 | (S)-Levodione (2) |

Experimental Procedures

All experiments were carried out in a glovebox (Glove Box Technology Ltd) under a protective N₂ atmosphere (O₂<0.1 ppm). Stock solutions of FAD and FMN were prepared using deoxygenated buffer. Different concentrations of stock solutions of 1 were prepared in DMSO such that DMSO was 1 vol % in the final reaction mixture.

General Procedure A (Flavin Reduction)

The indicated volume of Tris-HCl buffer (50 mM, pH 8.0) or phosphate buffer (50 mM, pH 8.0) was added to a UV-visible quartz cuvette, which was placed in the cell holder and allowed to warm to the indicated temperature (pre-set on the Peltier accessory) for 5 min. A baseline was recorded using the UV-visible spectrophotometer. A solution of 0.1 mM flavin (unless otherwise noted) in the designated buffer was next prepared in the cuvette, which was then capped with a rubber septum that was pierced with two needles to provide a gas inlet and outlet. An H₂-line was then connected and bubbled through the flavin solution via the inlet needle for 10 minutes. The needle was then moved up to the headspace through which a continuous H₂ flow was supplied. About 0.4 mL of the flavin solution was then used to transfer the designated quantity of Hyd1 into the cuvette using a syringe and needle, and the needle and syringe rinsed by drawing solution in and out of the cuvette. The assay was carried out by taking one scan (200-800 nm) every 30 seconds over 30 minutes.

General Procedure B (Alkene Reduction)

Using a syringe and needle, 600 μL of H₂-saturated Tris-HCl buffer (50 mM, pH 8.0, 25° C.) was transferred to a centrifuge tube (Eppendorf, 1.5 mL) that contained the required quantities of FMN and 1 in DMSO (1 vol % DMSO in total reaction mixture). A portion of this solution (approx. 0.2 mL) was used to transfer Hyd1 (57 μg, activated under H₂ for 3-15 h) and TsOYE (145 μg) into the reaction tube in sequence via a needle and syringe. The lid of the centrifuge tube was pierced once with a needle, capped, and placed in a Büchi Tinyclave pressure vessel which was then charged to the designated pressure of H₂. The pressure vessel was then removed from the glovebox and wrapped in aluminum foil to exclude light in order to prevent photodecomposition of the FMN, flavoenzyme, or both.^{[S6]} The vessel was placed on a Stuart® mini see-saw rocker set to 30 oscillations/min. The extent of conversion and enantiomeric excess (% ee) of (R)-2 was determined by chiral GC-FID (General Procedure C).

General Procedure C (Preparing Samples for Chiral GC Analysis)

Aliquots (25 μL) of reaction mixture were taken for analysis at 1 h and 15 h (and 24 h when indicated) then extracted into 200 μL EtOAc with 2 mM undecane as an internal standard. The biphasic solution was centrifuged to separate out any solids (12,000×g, 2 min), then 150 μL of the EtOAc layer was removed, dried over Na₂SO₄ and 75 μL of the solution was taken for GC analysis.

### Supplementary Data and Results

FIG. 9 shows current applications and methods of flavin recycling A. Examples of flavoenzymes applied toward natural products and analogues,^{[S7-9]} degradation of an environmental pollutant,^{[S10]} and a non-native light-driven cyclisation.^{[S11]} B. Current enzymatic flavin regeneration methods rely on NAD(P)H, which itself is continually regenerated using expensive, carbon-based sacrificial reductants. C. Other catalytic methods for flavin recycling tend to rely on cosubstrate additives. D. (This work) A simplified H₂-driven direct flavin reduction method using Hyd1 enzyme.

FIGS. 10 to 12 show control experiments to confirm role of Hyd1 and H₂ in flavin reduction:
FIG. 10 shows Background flavin reduction in absence of H₂. Reaction conditions: 800 μL scale, 0.1 mM flavin in Tris-HCl buffer (50 mM, pH 8, 25° C.), g Hyd1, 25° C. controlled by Peltier accessory. The Hyd1 specific activity for FAD and FMN reduction during this control reaction was 0.06 nmol min⁻¹ mg⁻¹ and 2.08 nmol min⁻¹ mg⁻¹ respectively.
FIG. 11 shows background flavin reduction in absence of Hyd1. Reaction conditions: 800 μL scale, 0.1 mM flavin in Tris-HCl buffer (50 mM, pH 8, 25° C.), H₂ flow (cuvette head space), 25° C. controlled by Peltier accessory. The overall decrease in [FAD] and [FMN] amounts to 0.000 mM and 0.005 mM after 30 minutes respectively.
FIG. 12 shows background flavin reduction in the absence of H₂ and Hyd1. Reaction conditions: 800 μL scale, 0.1 mM flavin in Tris-HCl buffer (50 mM, pH 8, 25° C.), 25° C. controlled by Peltier accessory. The overall decrease in [FAD] and [FMN] amounts to 0.000 mM and 0.080 mM after 30 minutes respectively.

Complete reduction of FMN to FMNH₂ was also demonstrated. FIG. 13 shows UV-visible spectra of FMN and FMNH₂ produced by Hyd1 under H₂ or sodium dithionite (gray).

Reaction conditions for FMN reduction by Hyd1: 800 μL scale, 0.1 mM FMN in Tris-HCl buffer (50 mM, pH 8, 25° C.), H₂ flow (cuvette head space), 57 μg Hyd1, 25° C. controlled by Peltier accessory. The full reduction of FMN by Hyd1 was completed during the experiment designed to test the stability of Hyd1 over time (>5 days).

Reaction conditions for FMN reduction by sodium dithionite (gray): 800 μL scale, 0.1 mM FMN in Tris-HCl buffer (50 mM, pH 8, 25° C.), 0.15 mM sodium dithionite, 25° C. controlled by Peltier accessory.

Control experiments for H₂-driven ketoisophorone reduction were also conducted. Control experiments were performed to see if Hyd1 (entry 1) or TsOYE (entry 2), alone, could lead to 2. In addition, similar experiments were done in the absence of FMN (entry 3) or no enzyme (entry 4). The control experiments demonstrated the need for each reaction component for the reaction to be successful. The results of the experiment are shown in Table 2.

TABLE 2
Control experiments for H₂-driven ketoisophorone reduction

| | | | | Conversion |
|---|---|---|---|---|
| Entry | FMN | TsOYE | Hyd1 | to 2 (%) |
| 1 | ✓ | — | ✓ | 0 |
| 2 | ✓ | ✓ | — | 0 |
| 3 | — | ✓ | ✓ | 0 |
| 4 | ✓ | — | — | 0 |

Reaction conditions: 600 μL scale, 0.1 mM FAD, 57 μg Hyd1, TsOYE (145 μg), 10 mM ketoisophorone, Tris-HCl buffer (50 mM, pH 8.0, 25° C.), 1 vol % DMSO at ambient temperature in pressure vessel (1 bar H₂), 24 h.

Exemplary chiral-phase GC-FID spectra of enzymatic H₂-driven reduction of ketoisophorone to (R)-levodione was demonstrated. Reduction of 1 (entry 4, Table 1) was carried out and the reaction mixture was analysed by chiral GC-FID according to General Procedure C. Conversion to 1 and the enantiomeric excess (% ee) were calculated based on the peak area of their respective peaks as shown in FIG. 14, which shows GC-FID results of ketoisophorone reductions (gray). Ketoisophorone, (rac)-levodione and (R)-levodione standards were diluted using EtOAc with 2 mM undecane as internal standard.

### Example 2

This example demonstrates further examples of flavin-cycling coupled to ene-reductions in accordance with the claimed invention. H₂ is used as a reductant with Hyd1 catalysing flavin reduction to allow catalytic reduction of the alkenes dimethyl itaconate and 4-phenyl-3-buten-2-one.

We extended the H₂-driven system described in Example 1 to a suite of commercially-available ene-reductases (Johnson Matthey). The alkene reductions demonstrated were dimethyl itaconate (3) reduction to dimethyl (R)-methyl succinate (4) and 4-phenyl-3-buten-2-one (5) reduction to 4-phenyl-2-butanone (6) (Table 1), using the same protocols established for TsOYE in Example 1. Control experiments demonstrated conversion in the presence of Hyd1, FMN, and ENE.

TABLE 3
H₂-driven enzymatic alkene reductions using commercial ene-reductases

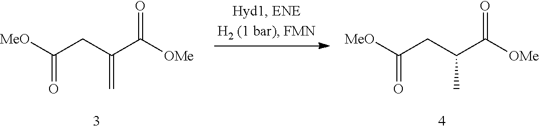

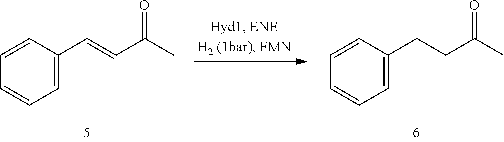

| | | [FMN] | Ene- | Time | Conv. | ee |
|---|---|---|---|---|---|---|
| Entry | Substrate | (mM) | reductase | (h) | (%)^{a} | (%) |
| 1 | 3 | 0.5 | ENE-101 | 17 | 75 | n.d.^{b} |
| 2 | 3 | 0.1 | ENE-103 | 42 | 81 | >99 |
| 3 | 3 | 0.5 | ENE-103 | 42 | 98 | >99 |
| 4 | 3 | 0.5 | ENE-108 | 17 | 99 | n.d.^{b} |
| 5 | 3 | 0.5 | ENE-109 | 17 | 100 | n.d.^{b} |
| 6 | 5 | 0.5 | ENE-103 | 17 | 100 | n.a.^{d} |
| 7^{c} | 5 | 0.1 | ENE-107 | 24 | 20 ± 1 | n.a.^{d} |
| 8^{c} | 5 | 0.5 | ENE-107 | 24 | 33 ± 3 | n.a.^{d} |
| 9 | 5 | 0.1 | ENE-107 | 40 | 35 | n.a.^{d} |
| 10 | 5 | 0.5 | ENE-107 | 40 | 100 | n.a.^{d} |
| 11 | 5 | 0.5 | ENE-108 | 17 | 100 | n.a.^{d} |
| 12 | 5 | 0.5 | ENE-109 | 17 | 100 | n.a.^{d} |

Reaction conditions: 23.7 μg/mL Hyd1, 0.5 mg/mL ene-reductase and 5 mM substrate in Tris-HCl (50 mM, pH 8), 1 vol % DMSO at room temperature (20-30° C.) in a sealed vessel under an H₂ (1 bar) atmosphere.
^{a}GC conversions to 4 or 6.
^{b}Not determined.
^{c}Entries 3 and 4 were performed in triplicate and are shown ±1 standard deviation, and were separate experiments from entries 5 and 6.
^{c}Not applicable.

High conversions to (R)-4 (measured by GC) were reached with ENE-101 (https://matthey.com/en/products-and-services/pharmaceutical-and-medical/catalysts/ene-101), -103 (https://matthey.com/en/products-and-services/pharmaceutical-and-medical/catalysts/ene-103), -108 (https://matthey.com/en/products-and-services/pharmaceutical-and-medical/catalysts/ene-108), and -109 (https://matthey.com/en/products-and-services/pharmaceutical-and-medical/catalysts/ene-109) (Table 3, entries 1-5). With ENE-103, we confirmed that enantioselective (>99% ee) reduction to (R)-4 was achieved using chiral-phase GC-FID (entries 2-3). Conversion of 5 to 6 was successful using ENE-103, -107 (https://matthey.com/en/products-and-services/pharmaceutical-and-medical/catalysts/ene-107), -108, and -109 (entries 6-12). Entries 7-8 were performed in triplicate in order to demonstrate the high reproducibility of the reaction.

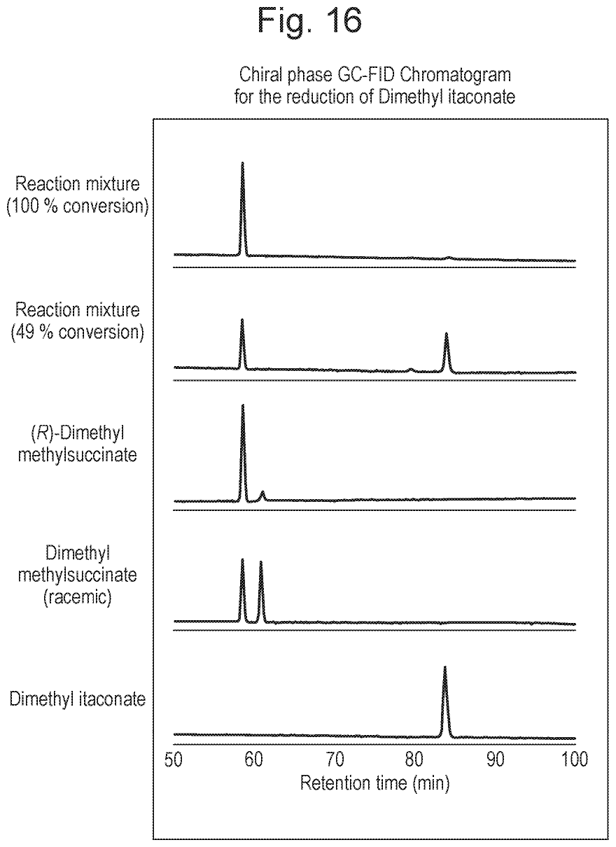

FIG. 15 shows characterisation data for the enzymatic reduction of 5 to 6 by GC-FID. FIG. 15 confirms that the methods of the invention can be used to reduce 5 to 6 with extremely high conversion efficiency.

Experimental conditions for the data collected in FIG. 15 were as follows:

Methods:

4-phenyl-3-buten-2-one (commercially available, 99%) and 4-phenyl-2-butanone (commercially available, 98%) standards were diluted using EtOAc.

GC Method:

Instrument: ThermoScientific Trace 1310 GC; Column: CP-Chirasil-Dex CB (Agilent), 25 m length, 0.25 mm diameter, 0.25 m (film thickness), fitted with a guard of 10 m undeactivated fused silica of the same diameter; Carrier: He (CP grade), 170 kPa (constant pressure); Inlet temperature: 200° C.; Injection conditions: Splitless with split flow 60 mL/min, splitless time 0.8 min, purge 5 mL/min. Injection volume=0.1 μL. Detection: FID (H₂=35 mL/min, air=350 mL/min, makeup N₂=40 mL/min, temp=200° C.)

Oven Heating Profile:

| Time (minutes) | Temperature |
|---|---|
| 0 → 5 | Hold at 70° C. |
| 5 → 30 | Ramp to 120° C. at 2° C./min |
| 30 → 36 | Ramp to 180° C. at 10° C./min |
| 36 → 45 | Hold at 180° C. for 5 minutes |

Compound Retention Times:

| Time (minutes) | Compound |
|---|---|
| 14.48 | 4-Phenyl-3-buten-2-one (5) |
| 12.86 | 4-Phenyl-2-butanone (6) |

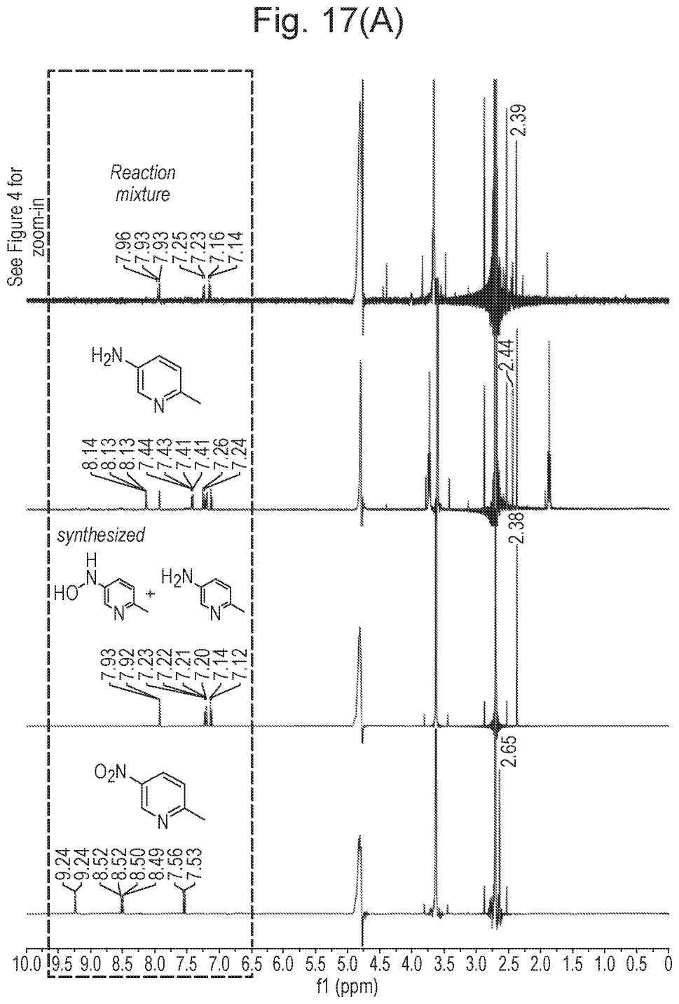

FIG. 16 shows characterisation data for the enzymatic reduction of 3 to 4 by GC-FID.

FIG. 15 confirms that the methods of the invention can be used to reduce 3 to 4 at with up to 100% conversion.

Experimental conditions for the data collected in FIG. 16 were as follows:

Methods:

GC-FID results of dimethyl itaconate reductions (top 2 panels). Dimethyl itaconate (commercially available, 98%), (rac)-dimethyl methyl succinate (commercially available, 98%) and dimethyl (R)-methyl succinate (commercially available, 99%) standards were diluted using EtOAc.

GC Method:

Instrument: ThermoFinnigan Trace GC; Column: Cyclosil-B (Agilent), 30 m length, 0.25 mm diameter, 0.25 m (film thickness); Carrier: He (CP grade), 100 kPa (constant pressure); Inlet temperature: 220° C.; Injection volume: 2 μL; Detection: FID (H₂₌₃₅ mL/min, air=350 mL/min, N2=30 mL/min, temp=250° C.

Oven Heating Profile:

| Time (minutes) | Temperature |
|---|---|
| 0 → 160 | Hold at 70° C. |
| 160 → 170 | Ramp to 180° C. at 20° C./min |

Compound Retention Times:

| Time (minutes) | Compound |
|---|---|
| 83.67 | Dimethyl itaconate (3) |
| 58.50 | (R)-Dimethyl methyl succinate (4) |
| 60.76 | (S)-Dimethyl methyl succinate (4) |

Example 2 thus confirms the broad applicability of the methods provided herein.

### Example 3

This example demonstrates further examples of flavin-cycling, coupled to other enzymatic reductions. In this example, H₂-driven flavin reduction was used to allow nitroreductases to reduce a nitro group (here 2-methyl-5-nitropyridine) to the corresponding amine, in accordance with the methods provided herein.

Hyd1-catalysed H₂-driven FMN and FAD recycling was coupled with nitroreductase (NR) enzymes (engineered, prepared and provided by Johnson Matthey; E. C. 1.7.1.16). These nitroreductase enzymes contain an FMN prosthetic group, and have been reported for use with GDH (glucose dehydrogenase)/glucose to continually supply a catalytic quantity of NADPH to the nitroreductase, which in turn will reduce aromatic nitro groups with the assistance of V₂O₅ as a co-catalyst (Scheme 1a).³⁸ However, such methods provide a mixture of the corresponding amine, hydroxylamine, and other undesired side products.

a. NR and Vanadium-Catalysed Nitro Reduction

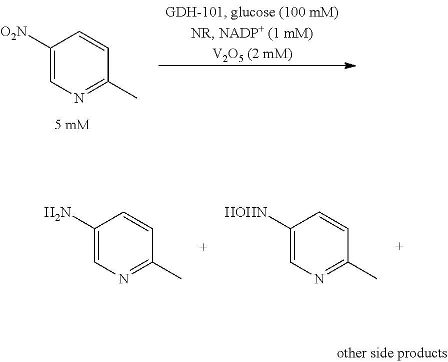

b. NR-Catalysed Nitro Reduction by H₂-Driven FMN Recycling (this Work)

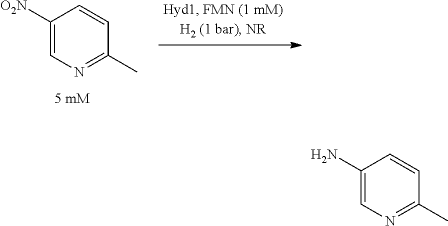

Scheme 1. Contrast between previously known methods of nitroreductase-catalysed nitro reductions and methods provided herein.

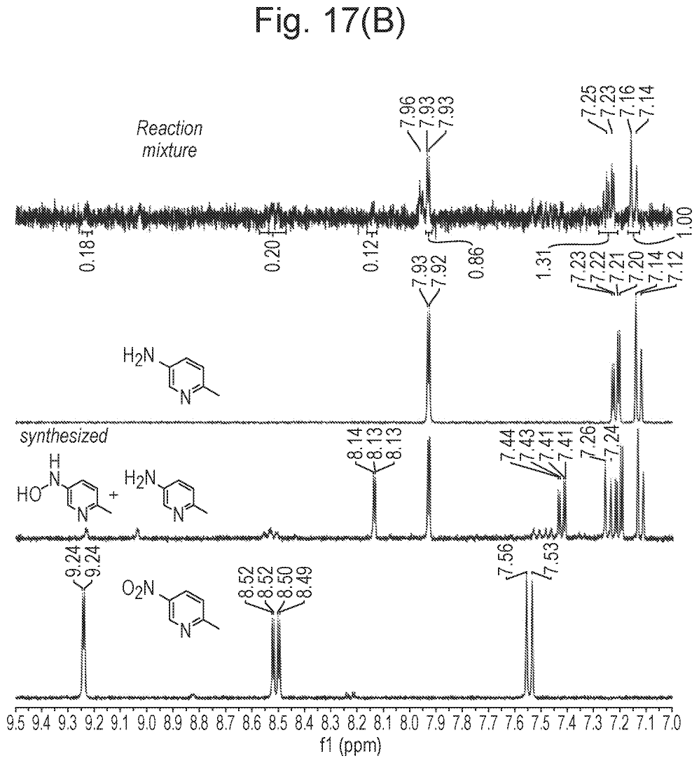

By contrast, the method provided herein for supplying an externally reduced FMN to OYE-type ene-reductases (see Example 1) can be extended for use other flavin reductases without generating unwanted side products. Here, we used Johnson Matthey nitroreductases (Scheme 1b). Thus, using the methods provided herein efficient conversions to the desired 5-amino-2-methylpyridine were confirmed using ¹H NMR spectroscopy, with comparison against commercially available authentic starting material and product standards. Over the time course of the reaction conducted, conversion of up to 70% was observed; longer reaction times would be expected to lead to more complete conversion. The hydroxylamine product was prepared following a known procedure,³⁹ which resulted in a mixture of amine and hydroxylamine products as confirmed by ¹H NMR spectroscopy (see FIG. 17).

Reactions conditions were as follows: 2-methyl-5-nitropyridine (5 mM), 1 mM FMN, Hyd1 (32 μg/mL) and NR (80 μg/mL) were dissolved in H₂-saturated Tris-HCl (50 mM, pH 8.0) with 2 vol % DMSO at room temperature, then left to react under an atmosphere (1 bar) of H₂ in a 40° C. water bath for 3 h.

¹H NMR spectra were obtained at room temperature on a Bruker Advance III HD nanobay (400 MHz) using water suppression. Samples was prepared with a total volume of 500 μL, containing 200 μL reaction solution, 250 μL deionised water, and 50 μL D₂O. Compound standards were prepared for NMR spectroscopic analysis using:

2-Methyl-5-aminopyridine: ¹H NMR (400 MHz, H₂O+D₂O, pH 8.0) δ 7.93 (d, J=2.8 Hz, 1H), 7.21 (dd, J=8.4, 2.8 Hz, 1H), 7.12 (d, J=8.4 Hz, 1H), 2.38 (s, 3H).

2-Methyl-5-hydroxylaminopyridine³⁹: ¹H NMR (400 MHz, H₂O+D₂O) δ 8.13 (d, J=2.7 Hz, 1H), 7.42 (dd, J=8.4, 2.7 Hz, 1H), 7.25 (d, J=8.4 Hz, 1H), 2.44 (s, 3H).

2-Methyl-5-nitropyridine: ¹H NMR (400 MHz, H₂O+D₂O, pH 8.0) δ 9.24 (d, J=2.7 Hz, 1H), 8.51 (dd, J=8.7, 2.7 Hz, 1H), 7.54 (d, J=8.7 Hz, 1H), 2.65 (s, 3H).

### Example 4

This example demonstrates flavin reduction by the methods of the invention using a range of other hydrogenase enzymes.

The reduction of flavin cofactors, FAD and FMN by hydrogenases different to Hyd1 was shown using Hydrogenase-2 (Hyd2) (PDB code: 6EHQ, EC 1.12.99.6—SEQ ID NOs: 3/4) from E. coli and the NiFe hydrogenase from Desulfovibrio vulgaris Miyazaki F (DvMF) (PDB code: 1WUJ, EC 1.12.2.1; SEQ ID NOs: 23/24)

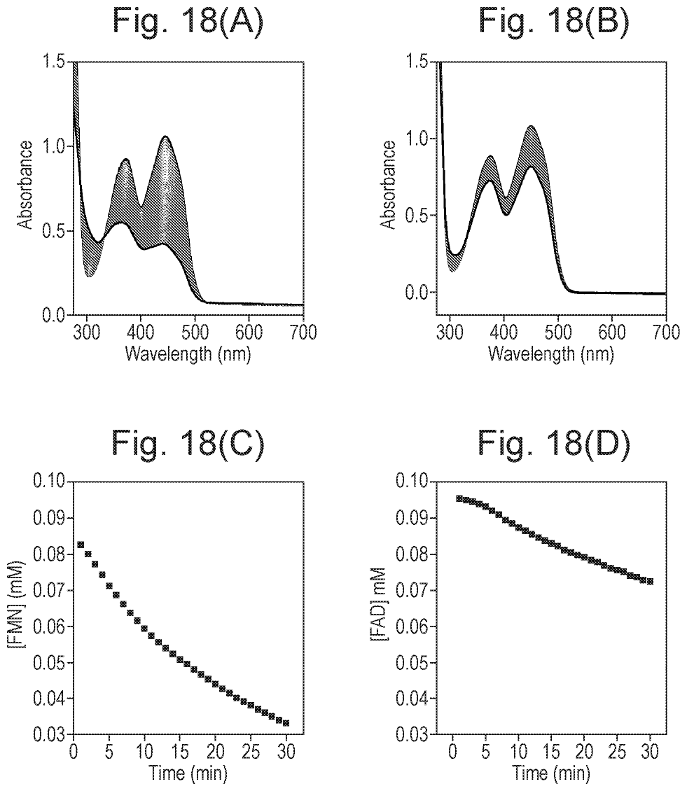

E. coli Hyd2 is relatively more oxygen sensitive than E. coli Hyd1 and works reversibly catalysing both H₂ oxidation and evolution efficiently. On supply of H₂, flavin reduction activity by Hyd2 was observed and the resulting UV-vis spectra showing flavin reduction are shown in FIG. 18. Activities were measured during the linear phase of the reaction. FIG. 18 shows activity assay results for Hyd2 catalysed reduction of A) FMN and B) FAD measured by in situ UV-visible Spectroscopy, C) calculated [FMN] based on λₘₐₓ=445 nm (ε=12.50 mM⁻¹ cm⁻¹), D) calculated [FAD] based on λₘₐₓ=450 nm (ε=11.30 mM⁻¹ cm⁻¹). Reaction conditions: 800 μL scale, 0.1 mM flavin, 0.048 mg/mL Hyd2 (activated under 1 bar H₂ for 20 h), TrisHCl buffer (pH 8), temp: 25° C. Hydrogenase specific activity was calculated as:

| | Hydrogenase |
|---|---|
| | specific activity |
| Cofactor | (nmol min⁻¹ mg⁻¹) |
| FMN | 31 |
| FAD | 17 |

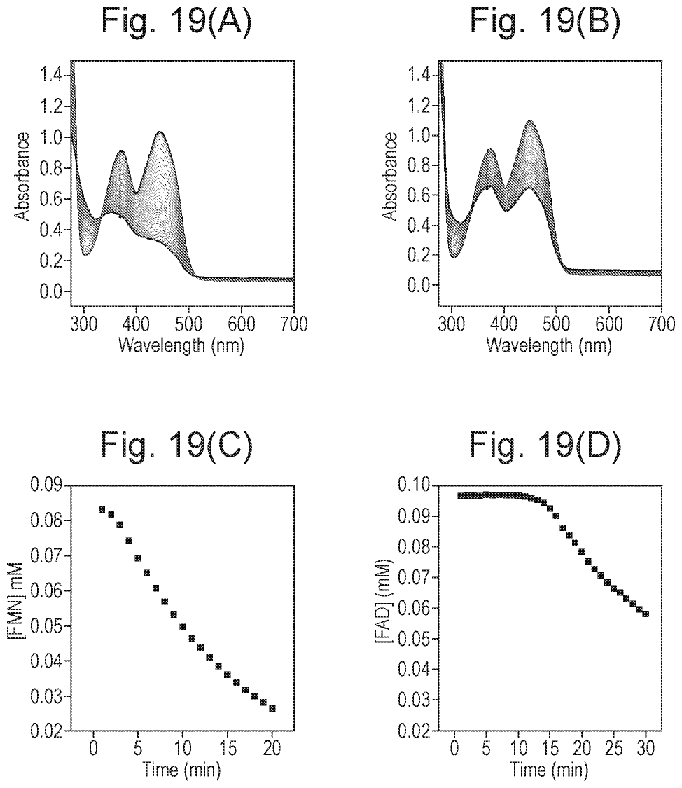

Similar reduction of flavins by Desulfovibrio vulgaris Miyazaki F (DvMF) NiFe hydrogenase was also observed and their activities calculated. FIG. 19 shows activity assay for DvMF catalysed reduction of A) FMN and B) FAD measured by in situ UV-visible Spectroscopy, C) calculated [FMN] based on λₘₐₓ=445 nm (ε=12.50 mM⁻¹ cm⁻¹), D) calculated [FAD] based on λₘₐₓ=450 nm (ε=11.30 mM⁻¹ cm⁻¹). Reaction conditions: 800 ul scale, 0.1 mM flavin, 0.045 mg/ml DvMF (activated under a flow of H₂ for 45 min), Tris HCl buffer (pH 8), temp: 25° C. Hydrogenase specific activity was calculated as:

| | Specific activity |
|---|---|
| Cofactor | (nmol min⁻¹ mg⁻¹) |
| FMN | 70 |
| FAD | 51 |

For FMN, only a 20 minute activity assay was performed, however, almost full conversion of FMN to FMNH₂ was observed during this reaction time. Similar to Hyd1, the flavin reduction activity was higher for FMN when compared to that of FAD during reduction reactions by both the enzymes.

Those skilled in the art will appreciate that a direct comparison of the reduction activities by the two enzymes (Hyd2 and DvMF) cannot be made, due to differences in techniques used for enzyme activation under hydrogen, and uncertainty with regard to the activation states and purities of the enzymes. However, the data presented in this example confirms that the methods of the invention are widely applicable to and are not restricted to specific hydrogenases.

### Example 5

This example demonstrates the solvent tolerance of the methods provided herein.

The specific activity of E. coli Hyd1 for FAD reduction was measured by UV/vis assay (recording A_{450 nm} over time, [symbol] _{FAD λ=450 nm)}=0.0113 mol⁻¹ dm³ cm⁻¹) at different mixtures of water:DMSO and water:acetonitrile.

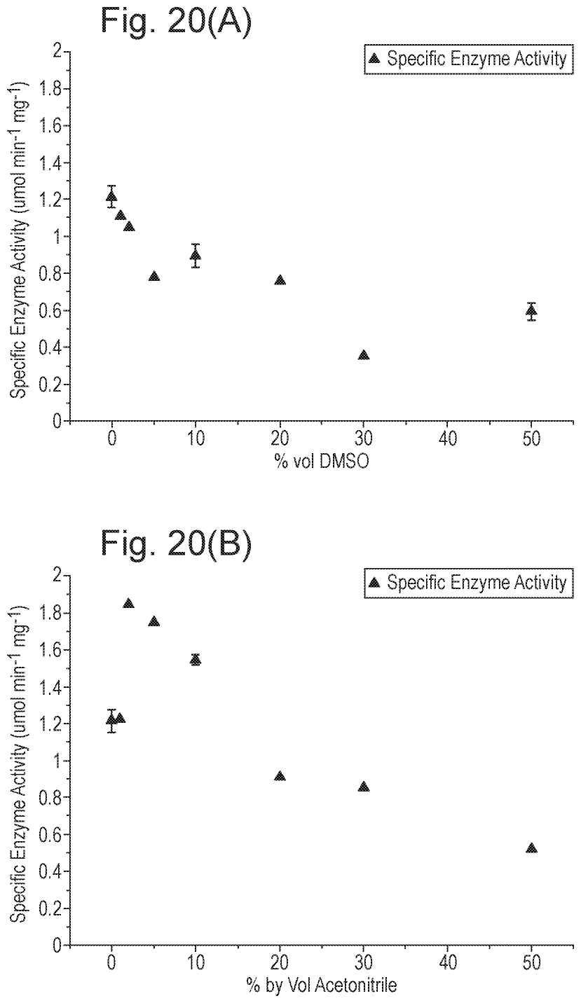

FIG. 20A shows the specific activity of E. coli Hyd1 for FAD reduction (pmol min⁻¹ mg⁻¹) measured at different mixtures of water: DMSO. The reaction mixture included 0.055 mg mL⁻¹ Hyd1, and 0.1 mM FAD under 1 bar H₂ at 25° C. Specific activity was measured from the initial linear phase of activity over approx. 20 minutes, following a brief lag phase corresponding to hydrogenase activation.

FIG. 20B shows the specific activity of E. coli Hyd1 for FAD reduction (μmol min⁻¹ mg⁻¹) measured at different mixtures of water:acetonitrile (CH₃CN). The reaction mixture included 0.055 mg mL⁻¹ Hyd1, and 0.1 mM FAD under 1 bar H₂ at 25° C. Specific activity was measured from the initial linear phase of activity over approx. 20 minutes, following a brief lag phase corresponding to hydrogenase activation.

The results in this example confirm that the methods of the invention are applicable to a range of reaction conditions including different ratios of solvent and water. Enzymatic reduction can be carried out even at elevated solvent levels.

### REFERENCES

[1] R. A. Sheldon, J. M. Woodley, Chem. Rev. 2018, 118, 801-838.
[2] K. Chen, F. H. Arnold, Nat. Catal. 2020, 3, 203-213.
[3] H. A. Bunzel, X. Garrabou, M. Pott, D. Hilvert, Curr. Opin. Struc. Biol. 2018, 48, 149-156.
[4] J. Latham, E. Brandenburger, S. A. Shepherd, B. R. K. Menon, J. Micklefield, Chem. Rev. 2018, 118, 232-269.
[5] C. K. Winkler, K. Faber, M. Hall, Curr. Opin. Biotechnol. 2018, 43, 97-105.
[6] R. D. Ceccoli, D. A. Bianchi, D. V. Rial, Front. Microbiol. 2014, 5, 25.
[7] S. A. Baker Dockrey, A. R. H. Narayan, Tetrahedron 2019, 75, 1115-1121.
[8] S. Adak, T. P. Begley, Biochemistry 2019, 58, 1181-1183.
[9] K. F. Biegasiewicz, S. J. Cooper, X. Gao, D. G. Oblinsky, J. H. Kim, S. E. Garfinkle, L. A. Joyce, B. A. Sandoval, G. D. Scholes, T. K. Hyster, Science (80-.).2019, 364, 1166-1169.
[10] H. S. Toogood, N. S. Scrutton, ACS Catal. 2018, 8, 3532-3549.
[11] F. Rudroff, M. D. Mihovilovic, H. Gröger, R. Snajdrova, H. Iding, U. T. Bornscheuer, Nat. Catal. 2018, 1, 12-22.
[12] L. Selles Vidal, C. L. Kelly, P. M. Mordaka, J. T. Heap, Biochim. Biophys. Acta Proteins Proteomics 2018, 1866, 327-347.
[13] M. Ismail, L. Schroeder, M. Frese, T. Kottke, F. Hollmann, C. E. Paul, N. Sewald, ACS Catal. 2019, 9, 1389-1395.
[14] J. P. Adams, M. J. B. Brown, A. Diaz-Rodriguez, R. C. Lloyd, G. Roiban, Adv. Synth. Catal. 2019, 361, 2421-2432.
[15] T. Heine, W. van Berkel, G. Gassner, K.-H. van Pée, D. Tischler, Biology (Basel). 2018, 7, 42.
[16] H. A. Reeve, L. Lauterbach, O. Lenz, K. A. Vincent, ChemCatChem 2015, 7, 3480-3487.
[17] L. Lauterbach, O. Lenz, K. A. Vincent, FEBS J. 2013, 280, 3058-3068.
[18] K. Schneider, H. G. Schlegel, Biochim. Biophys. Acta 1976, 452, 66-80.
[19] O. Lenz, L. Lauterbach, S. Frielingsdorf, in Methods Enzymol. (Ed.: F. A. Armstrong), Academic Press Inc., 2018, pp. 117-151.
[20] N. Herr, J. Ratzka, L. Lauterbach, O. Lenz, M. B. Ansorge-Schumacher, J Mol. Catal. B. Enzym. 2013, 97, 169-174.
[21] S. Vogt, M. Schneider, H. Scha Fer-Eberwein, G. Nö, Anal. Chem. 2014, 86, 7530-7535.
[22] P. Wulff, C. C. Day, F. Sargent, F. A. Armstrong, PNAS 2014, 111, 6606-6611.
[23] M. J. Lukey, A. Parkin, M. M. Roessler, B. J. Murphy, J. Harmer, T. Palmer, F. Sargent, F. A. Armstrong, J. Biol. Chem. 2010, 285, 3928-38.
[24] A. Volbeda, P. Amara, C. Darnault, J.-M. Mouesca, A. Parkin, M. M. Roessler, F. A. Armstrong, J. C. Fontecilla-Camps, PNAS 2012, 109, 5305-5310.
[25] R. M. Evans, E. J. Brooke, S. A. M Wehlin, E. Nomerotskaia, F. Sargent, S. B. Carr, S. E. V Phillips, F. A. Armstrong, Nat. Chem. Biol. 2015, 12, 46-50.
[26] R. Hidalgo, P. A. Ash, A. J. Healy, K. A. Vincent, Angew. Chem. Int. Ed. 2015, 54, 7110-7113.
[27] K. A. Vincent, A. Parkin, F. A. Armstrong, Chem. Rev. 2007, 107, 4366-4413.
[28] B. J. Murphy, F. Sargent, F. A. Armstrong, Energy Environ. Sci. 2014, 7, 1426-1433.
[29] C. Pinske, S. Kruger, B. Soboh, C. Ihling, M. Kuhns, M. Braussemann, M. Jaroschinsky, C. Sauer, F. Sargent, A. Sinz, et al., Arch. Microbiol. 2011, 193, 893-903.
[30] P. Macheroux, in Methods Mol. Biol. (Eds.: S. K. Chapman, G. A. Reid), Humana Press, Totowa, N.J., 1999, pp. 1-7.
[31] A. M. Edwards, in Methods Mol. Biol. (Eds.: S. Weber, E. Schleiker), Humana Press, New York, 2014, pp. 3-13.
[32] R. M. Evans, P. A. Ash, S. E. Beaton, E. J. Brooke, K. A. Vincent, S. B. Carr, F. A. Armstrong, J. Am. Chem. Soc. 2018, 140, 10208-10220.
[33] J. Bernard, E. van Heerden, I. W. C. E. Arends, D. J. Opperman, F. Hollmann, ChemCatChem 2012, 4, 196-199.
[34] D. J. Opperman, B. T. Sewell, D. Litthauer, M. N. Isupov, J. A. Littlechild, E. van Heerden, Biochem. Biophys. Res. Commun. 2010, 393, 426-431.
[35] B. R. K. Menon, J. Latham, M. S. Dunstan, E. Brandenburger, U. Klemstein, D. Leys, C. Karthikeyan, M. F. Greaney, S. A. Shepherd, J. Micklefield, Org. Biomol. Chem. 2016, 14, 9354-9361.
[36] J. Dong, E. Fernindez-Fueyo, F. Hollmann, C. E. Paul, M. Pesic, S. Schmidt, Y. Wang, S. Younes, W. Zhang, Angew. Chem. Int. Ed. 2018, 57, 9238-9261.
[37] T. A. Rogers, A. S. Bommarius, Chem. Eng. Sci. 2010, 65, 2118-2124.
[38] A. Bornadel et al. Org. Process Res. Dev. 2021, 25, 648-653.
[39] US 2020/0262784 A1.
[S1] S. Mathew, M. Trajkovic, H. Kumar, Q.-T. Nguyen, M. W. Fraaije, Chem. Commun. 2018, 54, 11208-11211.
[S2] H. A. Reeve, L. Lauterbach, O. Lenz, K. A. Vincent, ChemCatChem 2015, 7, 3480-3487.
[S3] D. Johannes Opperman, L. Ann Piater, E. van Heerden, J. Bacteriol. 2008, 190, 3076-3082.
[S4] D. J. Opperman, B. T. Sewell, D. Litthauer, M. N. Isupov, J. A. Littlechild, E. van Heerden, Biochem. Biophys. Res. Commun. 2010, 393, 426-431.
[S5] P. Macheroux, in Methods Mol. Biol. (Eds.: S. K. Chapman, G. A. Reid), Humana Press, Totowa, N.J., 1999, pp. 1-7.
[S6] M. C. R. Rauch, M. Pesic, M. M. E. Huijbers, M. Pabst, C. E. Paul, M. Pesid, I. W. C. E. Arends, F. Hollmann, BBA-Proteins Proteom. 2020, 1868, 140303.
[S7] R. A. Cacho, Y. H. Chooi, H. Zhou, Y. Tang, ACS Chem. Biol. 2013, 8, 2322-2330.
[S8] M. L. Contente, P. Zambelli, S. Galafassi, L. Tamborini, A. Pinto, P. Conti, F. Molinari, D. Romano, J. Mol. Catal. B.-Enzym. 2015, 114, 7-12.
[S9] S. W. Haynes, X. Gao, Y. Tang, C. T. Walsh, J. Am. Chem. Soc. 2012, 134, 17444-17447.
[S10] S. Adak, T. P. Begley, Biochemistry 2019, 58, 1181-1183.
[S11] K. F. Biegasiewicz, S. J. Cooper, X. Gao, D. G. Oblinsky, J. H. Kim, S. E. Garfinkle, L. A. Joyce, B. A. Sandoval, G. D. Scholes, T. K. Hyster, Science 2019, 364, 1166-1169.

SEQUENCE LISTING
SEQ ID NO: 1 - Escherichia coli hydrogenase 1 (large subunit).
MSTQYETQGYTINNAGRRLVVDPITRIEGHMRCEVNINDQNVITNAVSCGTMFRGLEIIL
QGRDPRDAWAFVERICGVCTGVHALASVYAIEDAIGIKVPDNANIIRNIMLATLWCHDHL
VHFYQLAGMDWIDVLDALKADPRKTSELAQSLSSWPKSSPGYFFDVQNRLKKFVEGGQLG
IFRNGYWGHPQYKLPPEANLMGFAHYLEALDFQREIVKIHAVFGGKNPHPNWIVGGMPCA
INIDESGAVGAVNMERLNLVQSIITRTADFINNVMIPDALAIGQFNKPWSEIGTGLSDKC
VLSYGAFPDIANDFGEKSLLMPGGAVINGDFNNVLPVDLVDPQQVQEFVDHAWYRYPNDQ
VGRHPFDGITDPWYNPGDVKGSDTNIQQLNEQERYSWIKAPRWRGNAMEVGPLARTLIAY
HKGDAATVESVDRMMSALNLPLSGIQSTLGRILCRAHEAQWAAGKLQYFFNKLMTNLKNG
NLATASTEKWEPTTWPTECRGVGFTEAPRGALGHWAAIRDGKIDLYQCVVPTTWNASPRD
PKGQIGAYEAALMNTKMAIPEQPLEILRTLHSFDPCLACSTHVLGDDGSELISVQVR
SEQ ID NO: 2 - Escherichia coli hydrogenase 1 (small subunit).
MNNEETFYQAMRRQGVTRRSFLKYCSLAATSLGLGAGMAPKIAWALENKPRIPVVWIHGL
ECTCCTESFIRSAHPLAKDVILSLISLDYDDTLMAAAGTQAEEVFEDIITQYNGKYILAV
EGNPPLGEQGMFCISSGRPFIEKLKRAAAGASAIIAWGTCASWGCVQAARPNPTQATSID
KVITDKPIIKVPGCPPIPDVMSAIITYMVTFDRLPDVDRMGRPLMFYGQRIHDKCYRRAH
FDAGEFVQSWDDDAARKGYCLYKMGCKGPTTYNACSSTRWNDGVSFPIQSGHGCLGCAEN
GFWDRGSFYSRVVDIPQMGTHSTADTVGLTALGVVAAAVGVHAVASAVDQRRRHNQQPTE
TEHQPGNEDKQA
SEQ ID NO: 3 - Escherichia coli hydrogenase 2 (large subunit).
MSQRITIDPVTRIEGHLRIDCEIENGVVSKAWASGTMWRGMEEIVKNRDPRDAWMIVQRICGVCTT
THALSSVRAAESALNIDVPVNAQYIRNIILAAHTTHDHIVHFYQLSALDWVDITSALQADPTKASE
MLKGVSTWHLNSPEEFTKVQNKIKDLVASGQLGIFANGYWGHPAMKLPPEVNLIAVAHYLQALECQ
RDANRVVALLGGKTPHIQNLAVGGVANPINLDGLGVLNLERLMYIKSFIDKLSDFVEQVYKVDTAV
IAAFYPEWLTRGKGAVNYLSVPEFPTDSKNGSFLFPGGYIENADLSSYRPITSHSDEYLIKGIQES
AKHSWYKDEAPQAPWEGTTIPAYDGWSDDGKYSWVKSPTFYGKTVEVGPLANMLVKLAAGRESTQN
KLNEIVAIYQKLTGNTLEVAQLHSTLGRIIGRTVHCCELQDILQNQYSALITNIGKGDHTTFVKPN
IPATGEFKGVGFLEAPRGMLSHWMVIKDGIISNYQAVVPSTWNSGPRNFNDDVGPYEQSLVGTPVA
DPNKPLEVVRTIHSFDPCMACAVH
SEQ ID NO: 4 - Escherichia coli hydrogenase 2 (small subunit).
MEMAESVTNPQRPPVIWIGAQECTGCTESLLRATHPTVENLVLETISLEYHEVLSAAFGHQVEENK
HNALEKYKGQYVLVVDGSIPLKDNGIYCMVAGEPIVDHIRKAAEGAAAIIAIGSCSAWGGVAAAGV
NPTGAVSLQEVLPGKTVINIPGCPPNPHNFLATVAHIITYGKPPKLDDKNRPTFAYGRLIHEHCER
RPHFDAGRFAKEFGDEGHREGWCLYHLGCKGPETYGNCSTLQFCDVGGVWPVAIGHPCYGCNEEGI
GFHKGIHQLANVENQTPRSQKPDVNAKEGGHHHHHH
SEQ ID NO: 5 - Ralstonia eutropha membrane-bound hydrogenase
moiety (HoxG).
MSAYATQGFNLDDRGRRIVVDPVTRIEGHMRCEVNVDANNVIRNAVSTGTMWRGLEVILK
GRDPRDAWAFVERICGVCTGCHALASVRAVENALDIRIPKNAHLIREIMAKTLQVHDHAV
HFYHLHALDWVDVMSALKADPKRTSELQQLVSPAHPLSSAGYFRDIQNRLKRFVESGQLG
PFMNGYWGSKAYVLPPEANLMAVTHYLEALDLQKEWVKIHTIFGGKNPHPNYLVGGVPCA
INLDGIGAASAPVNMERLSFVKARIDEIIEFNKNVYVPDVLAIGTLYKQAGWLYGGGLAA
TNVLDYGEYPNVAYNKSTDQLPGGAILNGNWDEVFPVDPRDSQQVQEFVSHSWYKYADES
VGLHPWDGVTEPNYVLGANTKGTRTRIEQIDESAKYSWIKSPRWRGHAMEVGPLSRYILA
YAHARSGNKYAERPKEQLEYSAQMINSAIPKALGLPETQYTLKQLLPSTIGRTLARALES
QYCGEMMHSDWHDLVANIRAGDTATANVDKWDPATWPLQAKGVGTVAAPRGALGHWIRIK
DGRIENYQCVVPTTWNGSPRDYKGQIGAFEASLMNTPMVNPEQPVEILRTLHSFDPCLAC
STHVMSAEGQELTTVKVR
SEQ ID NO: 6 - Ralstonia eutropha membrane-bound hydrogenase
moiety (HoxK).
MVETFYEVMRRQGISRRSFLKYCSLTATSLGLGPSFLPQIAHAMETKPRTPVLWLHGLEC
TCCSESFIRSAHPLAKDVVLSMISLDYDDTLMAAAGHQAEAILEEIMTKYKGNYILAVEG
NPPLNQDGMSCIIGGRPFIEQLKYVAKDAKAIISWGSCASWGCVQAAKPNPTQATPVHKV
ITDKPIIKVPGCPPIAEVMTGVITYMLTFDRIPELDRQGRPKMFYSQRIHDKCYRRPHFD
AGQFVEEWDDESARKGFCLYKMGCKGPTTYNACSTTRWNEGTSFPIQSGHGCIGCSEDGF
WDKGSFYDRLTGISQFGVEANADKIGGTASVVVGAAVTAHAAASAIKRASKKNETSGSEH
SEQ ID NO: 7 - Ralstonia eutropha membrane-bound hydrogenase
moiety (HoxZ).
MSTKMQADRIADATGTDEGAVASGKSIKATYVYEAPVRLWHWVNALAIVVLAVTGFFIGS
PPATRPGEASANFLMGYIRFAHFVAAYIFAIGMLGRIYWATAGNHHSRELFSVPVFTRAY
WQEVISMLRWYAFLSARPSRYVGHNPLARFAMFFIFFLSSVFMILTGFAMYGEGAQMGSW
QERMFGWVIPLLGQSQDVHTWHHLGMWFIVVFVIVHVYAAIREDIMGRQSVVSTMVSGYR
TFKD
SEQ ID NO: 8 - Ralstonia eutropha regulatory hydrogenase moiety
(HoxB).
MNAPVCTGLASAKPGVLNVLWIQSGGCGGCSMSLLCADTTDFTGMLKSAGIHMLWHPSLS
LESGVEQLQILEDCLQGRVALHALCVEGAMLRGPHGTGRFHLLAGTGVPMIEWVSRLAAV
ADYTLAVGTCAAYGGITAGGGNPTDACGLQYEGDQPGGLLGLNYRSRAGLPVINVAGCPT
HPGWVTDALALLSARLLTASDLDTLGRPRFYADQLVHHGCTRNEYYEFKASAEKPSDLGC
MMENMGCKGTQAHADCNTRLWNGEGSCTRGGYACISCTEPGFEEPGHPFHQTPKVAGIPI
GLPTDMPKAWFVALASLSKSATPKRVKLNATADHPLIAPAIRKTRLK
SEQ ID NO: 9 - Ralstonia eutropha regulatory hydrogenase moiety
(HoxC).
MERLVVGPFNRVEGDLEVNLEVASGRVCSARVNATMYRGLEQILLHRHPLDALVYAPRVC
GICSVSQSVAASRALADLAGVTVPANGMLAMNLMLATENLADHLTHFYLFFMPDFTREIY
AGRPWHTDATARFSPTHGKHHRLAIAARQRWFTLMGTLGGKWPHTESVQPGGSSRAIDAA
ERVRLLGRVREFRCFLEQTLYAAPLEDVVALDSEVALWRWHAQAPQAGDLRCFLTIAQDA
ALDQMGPGPGTYLSYGAYPQPEGGFCFAQGVWRSAQGRLDALDLAAISEDATSAWLVDQG
GARHPANGLTAPAPDKVGAYTWNKAPRLAGAVLETGAIARQLAGAQPLVRDAVARCGATV
YTRVLARLVELARVVPLMEDWLQSLEIGAPYWASAHLPDQGAGVGLTEAARGSLGHWVSV
RDGRIDNYQIVAPTSWNFSPRDIAGQPGAVEKALEGAPVLQGETTPVAVQHIVRSFDPCM
VCTVH
SEQ ID NO: 10 - Aquifex aeolicus hydrogenase 1 (large subunit).
MKRVVVDPVTRIEGHLRIEIMVDEETGQVKDALSAGTMWRGIELIVRNRDPRDVWAFTQR
ICGVCTSIHALASLRAVEDALEITIPKNANYIRNIMYGSLQVHDHVVHFYHLHALDWVSP
VEALKADPVATAALANKILEKYGVLNEFMPDFLGHRAYPKKFPKATPGYFREFQKKIKKL
VESGQLGIFAAHWWDHPDYQMLPPEVHLIGIAHYLNMLDVQRELFIPQVVFGGKNPHPHY
IVGGVNCSISMDDMNAPVNAERLAVVEDAIYTQVESTDFFYIPDILAIADIYLNQHNWFY
GGGLSKKRVIGYGDYPDEPYTGIKNGDYHKKILWHSNGVVEDFYKGVEKAKFYNLEGKDF
TDPEQIQEFVTHSWYKYPDETKGLHPWDGITEPNYTGPKEGTKTHWKYLDENGKYSWIKA
PRWRGKACEVGPLARYIIVYTKVKQGHIKPTWVDELIVNQIDTVSKILNLPPEKWLPTTV
GRTIARALEAQMSAHTNLYWMKKLYDNIKAGDTSVANMEKWDPSTWPKEAKGVGLTEAPR
GALGHWVIIKDGKVANYQCVVPTTWNGSPKDPKGQHGAFEESMIDTKVKVPEKPLEVLRG
IHSFDPCLACSTHLYNEKGEEIASVRVQGVVHV
SEQ ID NO: 11 - Aquifex aeolicus hydrogenase 1 (small subunit).
METFWEVFKRHGVSRRDFLKFATTITGLMGLAPSMVPEVVRAMETKPRVPVLWIHGLECT
CCSESEIRSATPLASDVVLSMISLEYDDTLSAAAGEAVEKHRERIIKEYWGNYILAVEGN
PPLGEDGMYCIIGGRPFVEILKESAEGAKAVIAWGSCASWGCVQAAKPNPTTAVPIDKVI
KDKPIIKVPGCPPIAEVMTGVIMYMVLFDRIPPLDSQGRPKMFYGNRIHDTCYRRSFFNA
GQFVEQFDDEGAKKGWCLYKVGCRGPTTYNSCGNMRWYNGLSYPIQSGHGCIGCAENNFW
DNGPFYERIGGIPVPGIESKADKVGAIAAAAAAGGAIIHGIASKIRKSGEKEE
SEQ ID NO: 12 - Hydrogenovibrio marinus hydrogenase (large
subunit).
MSVLNTPNHYKMDNSGRRVVIDPVTRIEGHMRCEVNVDENNVIQNAVSTGTMWRGLEVIL
RGRDPRDAWAFVERICGVCTGCHALASVRAVEDALDIKIPHNATLIREIMAKTLQIHDHI
VHFYHLHALDWVNPVNALKADPQATSELQKLVSPHHPMSSPGYFKDIQIRIQKFVDSGQL
GIFKNGYWSNPAYKLSPEADLMAVTHYLEALDFQKEIVKIHAIFGGKNPHPNYMVGGVPC
AINIDGDMAAGAPINMERLNFVKSLIEQGRTFNTNVYVPDVIAIAAFYRDWLYGGGLSAT
NVMDYGAYPKTPYDKSTDQLPGGAIINGDWGKIHPVDPRDPEQVQEFVTHSWYKYPDETK
GLHPWDGITEPNYELGSKTKGSRTNIIEIDESAKYSWIKSPRWRGHAVEVGPLARYILAY
AQGVEYVKTQVHTSLNRFNAVCRLLDPNHKDITDLKAFLGSTIGRTLARALESEYCGDMM
LDDFNQLISNIKNGDSSTANTDKWDPSSWPEHAKGVGTVAAPRGALAHWIVIEKGKIKNY
QCVVPTTWNGSPRDPKGNIGAFEASLMGTPMERPDEPVEVLRTLHSFDPCLACSTHVMSE
EGEEMATVKVR
SEQ ID NO: 13 - Hydrogenovibrio marinus hydrogenase (small
subunit).
MSSQVETFYEVMRRQGITRRSFLKYCSLTAAALGLSPAYANKIAHAMETKPRTPVIWLHG
LECTCCSESFIRSAHPLAKDVVLSMISLDYDDTLMAASGHAAEAILDEIKEKYKGNYILA
VEGNPPLNQDGMSCIIGGRPFSEQLKRMADDAKAIISWGSCASWGCVQAAKPNPTQATPV
HKFLGGGYDKPIIKVPGCPPIAEVMTGVITYMLTFDRIPELDRQGRPKMFYSQRIHDKCY
RRPHFDAGQFVEEWDDEGARKGYCLYKVGCKGPTTYNACSTVRWNGGTSFPIQSGHGCIG
CSEDGFWDKGSFYSRDTEMNAFGIEATADDIGKTAIGVVGAAVVAHAAISAVKAAQKKGD
K
SEQ ID NO: 14 - Thiocapsa roseopersicina hydrogenase HupL.
MSVTTANGFELDTAGRRLVVDPVTRIEGHLRCEVNLDENNVIRNAVSTGTMWRGLEVILR
GRDPRDAWAETERICGVCTGTHALTSVRAVEDALGIPIPENANSIRNIMHVTLQAHDHLV
HFYHLHALDWVDVVSALGADPKATSALAQSISDWPKSSPGYFRDVQNRLKRFVESGQLGP
FMNGYWGSPAYKLPPEANLMAVTHYLEALDFQKEIVKIHTVYGGKNPHPNWLVGGMPCAI
NVDGTGAVGAINMERLNLVSSIIDQTIAFIDKVYIPDLIAIASFYKDWTYGGGLSSQAVM
SYGDIPDHANDMSSKNLLLPRGAIINGNLNEIHEIDLRNPEEIQEFVDHSWFSYKDETRG
LHPWDGVTEPNFVLGPNAVGSRTRIEALDEQAKYSWIKAPRWRGHAMEVGPLARYVIGYA
KGIPEFKEPVDKVLTDLGQPLEAIFSTLGRTAARGLEASWAAHKMRYFQDKLVANIRAGD
TATANVDNWDPKTWPKEARGVGTTEAPRGALGHWIVIKDGKIDNYQAVVPTTWNGSPRDP
AGNIGAFEASLLNTPLAKADEPLEILRTLHSFDPCLACATHIMGPDGEELTRIKVR
SEQ ID NO: 15 - Thiocapsa roseopersicina hydrogenase HupS.
MPTTETYYEVMRRQGITRRSFLKFCSLTATALGLSPTFAGKIAHAMETKPRIPVVWLHGL
ECTCCSESFIRSAHPLVSDVILSMISLDYTILIMAAAGHQAEAILEEVRHKHAGNYILAV
EGNPPLNQDGMSCIIGGRPFLEQLLEMADSCKAVISWGSCASWGCVQAARPNPTRATPVH
EVIRDKPVIKVPGCPPIAEVMTGVLTYILTFDRLPELDRQGRPLMFYGQRIHDKCYRRPH
FDAGQFVESWDDEGARRGYCLYKVGCKGPTTYNACSTIRWNGGVSFPIQSGHGCIGCSED
GFWDKGSFYQHVTDTHAFGIEANADRTGIAVATRRGAAHRAHAAVSVVKRVQQKKEEDQS
SEQ ID NO: 16 - Alteromonas macleodii hydrogenase small subunit
MALPTLNKQLQASGISRRTFLKFCATTASLLALPQSAVADLATALGNARRPSVIWLPFQE
CTGCTEAILRSHAPTLESLIFDHISLDYQHTIMAAAGEQAEDARRAAMNAHKGQYLLLVD
GSVPVGNPGYSTISGMSNVDMLRESAKDAAGIIAIGTCASFGGIPKANPNPTGAVAVSDI
ITDKPIVNISGCPPLPIAITAVLVHYLTFKRFPDLDELQRPLAFFGESIHDRCYRRPFFE
QRKFAKSFDDEGAKNGWCLFELGCKGPETFNACATVKWNQGTSFPIESGHPCLGCSEPDF
WDKSSFYQALGPWEWYKSKPGKGAQKHAGKNS
SEQ ID NO: 17 - Alteromonas macleodii hydrogenase large subunit.
MENTASNNRLVVDPITRIEGHLRIEAEMDGNTIKQAFSSGTSVRGIELILQGRDPRDAWA
FAQRICGVCTLVHGMASVRAVEDAIRKAWRSNAKLGVAIGKPSMTSMPKGPMQHGKKGHR
QSRTSIGVLSEAEMAIPQNAQLIRNIMIATQYVHDHVMHFYHLHALDWVDVVSALDADPT
RTATLAGQLSDYPRSSPGYFKDVKQKVKTLVESGQLGIFSNAYWGHPGYKLPPEVNLMAL
AHYLDALTWQREVVKVHTIFGGKNPHPNFVVGGVPSPINLNASTGINTSRLVQLQDAITQ
MKSFVDQVYYPDIVAIAGYYKEWGTRGEGLGNFLTYGDLPMTSMDDPDSFLFPRGAILGR
DLSKVHDLDLDDPSEIQEFVSSSWYRYSGGNASGLHPFNGQTTLEYTGPKPPYKHLNVGA
EYSWLKSPRWKGHAMEVGPLARVLMMYAKKDAAAQDIVNRSLSILDLETSALFSTLGRTL
ARAVETKIVVNQLQSWYDQLLDNIAKGDTDTFNPLYFDPTNWPIKGQGVGVMEAPRGALG
HWLVMQNGKIENYQCVVPTTWNAGPRDPNSQAGAYEAALQDKHTLHDPDQPLEILRTLHS
FDPCLACAVHVMDETGEERLRLKVR
SEQ ID NO: 18 - Allochromatium vinosum Membrane Bound Hydrogenase
large subunit.
MSERIVVDPVTRIEGHLRIEAQMDGENIAQAYSSGTSVRGLETILKGRDPRDAWAFAQRI
CGVCTLVHGIASVRSVEDALKIELPPNAQLIRNLMISSQFVHDHVMHFYHLHALDWVDVV
SALSADPKATSDLAQSISSWPKSSPGYFADTQKRIKTFVESGQLGIFANGYWGHPAYKLP
PEANLMAVAHYLEALAWQRDVARLHAIFGGKNPHPNFVVGGVPSPIDIDSDSAINAKRLA
EVQQILQSMQTFVDQVYVPDTLAIASFYKDWGERGEGLGNFMSYGDLPATGTMDPAQFLF
PRGVILNRDLSTIHEIDLHDAGQIQEYVAHSWYEYSGGNDQGLHPYDGETNLEYDARGGV
KPPYTQLDVNDGYSWMKAPRWKGHAMEVGPLARVLLLYASGHEQTKELVEMTLTTLDLPV
RALYSTLGRTAARTLETKILTDTAQDWYNQLIANIKAGDSRTFNETLWEPSSWPAEARGA
GYMEAPRGALGHWIVIKDRKIANYQAVVPSTWNAGPRDPSDQPGAYEAALQDNHQLVDVK
QPIEILRTIHSFDPCIACAVHLTDPETGEQMEIKIT
SEQ ID NO: 19 - Allochromatium vinosum Membrane Bound Hydrogenase
small subunit.
PSVVWLSFQECTGCTESLTRAHAPTLEDLILDFISLDYHHTLQAASGEAAEAARLQAMDE
NRGQYLVIVDGSIPGPDANPGFSTVAGHSNYSILMETVEHAAAVIAVGTCAAFGGLPQAR
PNPTGAMSVMDLVRDKPVINVPGCPPIPMVITGVIAHYLVFGRLPEVDGYGRPLAFYGQS
IHDRCYRRPFYDKGLFAESFDDEGAKQGWCLYRLGCKGPTTYNACATMKWNDGTSWPVEA
GHPCLGCSEPQFWDAGGFYEPVSVPLTLGPATLLGAGAAGAVVGGGLAALSRKKGRDAAA
TRQPVTVDELEQKL
SEQ ID NO: 20 - Salmonella enterica serovar Typhimurium LT2
nickel-iron hydrogenase 5 Large subunit.
MAYPYQTQGFTLDNSGRRIVVDPVTRIEGHMRCEVNIDSNNVITNAVSTGTMWRGLEVIL
KGRDPRDAWAFVERICGVCTGTHALTSIRAVENALGIAIPDNANCIRNMMQATLHVHDHL
VHFYHLHALDWVDVVAALKADPHQTSAIAQSLSAWPLSSPGYFRDLQNRLKRFIESGQLG
PFRNGYWGHPAMKLPPEANLLAVAHYLEALDFQKEIVKIHTVFGGKNPHPNWLVGGVPCA
INLDETGAVGAVNMERLNLVSSIIQKARQFCEQVYLPDVLLIASYYKDWAKIGGGLSSMN
LLAYGEFPDNPNDYSASNLLLPRGAIINGRFDEIHPVDLTAPDEIQEFVTHSWYTYGNGN
NDKGLHPWDGLTEPQLVMGEHYKGTKTFIEQVDESAKYSWIKSPRWKGHAMEVGPLARYL
IGYHQNKPEFKEPVDQLLSVLKLPKEALFSTLGRTAARALESVWAGNTLQYFFDRLMRNL
KSGDTATANVTLWEPDTWPTSAKGVGFSEAPRGALGHWIKIANQKIDSYQCVVPTTWNAG
PRDDKGQIGAYEAALMGTKLAVPDQPLEILRTLHSFDPCLACSTH
SEQ ID NO: 21 - Salmonella enterica serovar Typhimurium LT2
nickel-iron hydrogenase 5 Small subunit.
LENKPRTPVIWLHGLECTCCTESFIRSAHPLAKDAILSLISLDYDDTIMAAAGQQAEQAL
ADVMREYKGNYIVAVEGNAPLNEDGMFCILAGEPFLEKLKRVSADAKAIIAWGSCASWGC
VQAARPNPTKATPVHKLITDKPIIKVPGCPPIPEVMSAVITYMLAFDRIPPLDRLGRPKM
FYGQRIHDKCYRRAHFDAGQFVEAWDDEGARKGYCLYKMGCKGPTTYNACSTVRWNDGVS
FPIQSGHGCLGCSEDGFWDYGSFYSRATGSRSHHHHHHH
SEQ ID NO: 22 Escherichia coli cytochrome HyaC.
MQQKSDNVVSHYVFEAPVRIWHWLTVLCMAVLMVTGYFIGKPLPSVSGEATYLFYMGYIRLIHFSA
GMVFTVVLLMRIYWAFVGNRYSRELFIVPVWRKSWWQGVWYEIRWYLFLAKRPSADIGHNPIAQAA
MFGYFLMSVFMIITGFALYSEHSQYAIFAPFRYVVEFFYWTGGNSMDIHSWHRLGMWLIGAFVIGH
VYMALREDIMSDDTVISTMVNGYRSHKFGKISNKERS
SEQ ID NO: 23 - Desulfovibrio vulgaris Miyazaki F hydrogenase
(large subunit).
SSYSGPIVVDPVTRIEGHLRIEVEVENGKVKNAYSSSTLFRGLEIILKGRDPRDAQHFTQRTCGVC
TYTHALASTRCVDNAVGVHIPKNATYIRNLVLGAQYLHDHIVHFYHLHALDFVDVTAALKADPAKA
AKVASSISPRKTTAADLKAVQDKLKTFVETGQLGPFTNAYFLGGHPAYYLDPETNLIATAHYLEAL
RLQVKAARAMAVFGAKNPHTQFTVVGGVTCYDALTPQRIAEFEALWKETKAFVDEVYIPDLLVVAA
AYKDWTQYGGTDNFITFGEFPKDEYDLNSRFFKPGVVFKRDFKNIKPFDKMQIEEHVRHSWYEGAE
ARHPWKGQTQPKYTDLHGDDRYSWMKAPRYMGEPMETGPLAQVLIAYSQGHPKVKAVTDAVLAKLG
VGPEALFSTLGRTAARGIETAVIAEYVGVMLQEYKDNIAKGDNVICAPWEMPKQAEGVGFVNAPRG
GLSHWIRIEDGKIGNFQLVVPSTWTLGPRCDKNKLSPVEASLIGTPVADAKRPVEILRTVHSFDPC
IACGVH
SEQ ID NO: 24 - Desulfovibrio vulgaris Miyazaki F hydrogenase
(small subunit).
LMGPRRPSVVYLHNAECTGCSESVLRAFEPYIDTLILDTLSLDYHETIMAAAGDAAEAALEQAVNS
PHGFIAVVEGGIPTAANGIYGKVANHTMLDICSRILPKAQAVIAYGTCATFGGVQAAKPNPTGAKG
VNDALKHLGVKAINIAGCPPNPYNLVGTIVYYLKNKAAPELDSLNRPTMFFGQTVHEQCPRLPHFD
AGEFAPSFESEEARKGWCLYELGCKGPVTMNNCPKIKFNQTNWPVDAGHPCIGCSEPDFWDAMTPF
YQN
SEQ ID NO: 31 - Chromate Reductase, ‘TsOYE’ from Thermus
scotoductus.
MALLFTPLELGGLRLKNRLAMSPMCQYSATLEGEVTDWHLLHYPTRALGGVGLILVEATA
VEPLGRISPYDLGIWSEDHLPGLKELARRIREAGAVPGIQLAHAGRKAGTARPWEGGKPL
GWRVVGPSPIPFDEGYPVPEPLDEAGMERILQAFVEGARRALRAGFQVIELHMAHGYLLS
SFLSPLSNQRTDAYGGSLENRMRFPLQVAQAVREVVPRELPLFVRVSATDWGEGGWSLED
TLAFARRLKELGVDLLDCSSGGVVLRVRIPLAPGFQVPFADAVRKRVGLRTGAVGLITTP
EQAETLLQAGSADLVLLGRVLLRDPYFPLRAAECALGVAPEVPPQYQRGF
SEQ ID NO: 32 - NADPH Dehydrogenase 1, ‘OYE-1’, Saccharomyces
pastorianus.
MSFVKDFKPQALGDTNLFKPIKIGNNELLHRAVIPPLTRMRALHPGNIPNRDWAVEYYTQ
RAQRPGTMIITEGAFISPQAGGYDNAPGVWSEEQMVEWTKIFNAIHEKKSFVWVQLWVLG
WAAFPDNLARDGLRYDSASDNVFMDAEQEAKAKKANNPQHSLTKDEIKQYIKEYVQAAKN
SIAAGADGVEIHSANGYLLNQFLDPHSNTRTDEYGGSIENRARFTLEVVDALVEAIGHEK
VGLRLSPYGVFNSMSGGAETGIVAQYAYVAGELEKRAKAGKRLAFVHLVEPRVTNPFLTE
GEGEYEGGSNDFVYSIWKGPVIRAGNFALHPEVVREEVKDKRTLIGYGRFFISNPDLVDR
LEKGLPLNKYDRDTFYQMSAHGYIDYPTYEEALKLGWDKK
SEQ ID NO: 33: NADPH Dehydrogenase 2, ‘OYE-2’, Saccharomyces
cerevisiae strain ATCC 204508/S288c.
MPFVKDFKPQALGDTNLFKPIKIGNNELLHRAVIPPLTRMRAQHPGNIPNRDWAVEYYAQ
RAQRPGTLIITEGTFPSPQSGGYDNAPGIWSEEQIKEWTKIFKAIHENKSFAWVQLWVLG
WAAFPDTLARDGLRYDSASDNVYMNAEQEEKAKKANNPQHSITKDEIKQYVKEYVQAAKN
SIAAGADGVEIHSANGYLLNQFLDPHSNNRTDEYGGSIENRARFTLEVVDAVVDAIGPEK
VGLRLSPYGVFNSMSGGAETGIVAQYAYVLGELERRAKAGKRLAFVHLVEPRVTNPFLTE
GEGEYNGGSNKFAYSIWKGPIIRAGNFALHPEVVREEVKDPRTLIGYGRFFISNPDLVDR
LEKGLPLNKYDRDTFYKMSAEGYIDYPTYEEALKLGWDKN
SEQ ID NO: 34: NADPH Dehydrogenase, ‘YqjM’, Bacillus subtilis.
MARKLFTPITIKDMTLKNRIVMSPMCMYSSHEKDGKLTPFHMAHYISRAIGQVGLIIVEASAVNPQ
GRITDQDLGIWSDEHIEGFAKLTEQVKEQGSKIGIQLAHAGRKAELEGDIFAPSAIAFDEQSATPV
EMSAEKVKETVQEFKQAAARAKEAGFDVIEIHAAHGYLIHEFLSPLSNHRTDEYGGSPENRYRFLR
EIIDEVKQVWDGPLFVRVSASDYTDKGLDIADHIGFAKWMKEQGVDLIDCSSGALVHADINVFPGY
QVSFAEKIREQADMATGAVGMITDGSMAEEILQNGRADLIFIGRELLRDPFFARTAAKQLNTEIPA
PVQYERGW
SEQ ID NO: 35: Xenobiotic Reductase A, ‘XenA’, Pseudomonas putida.
MSALFEPYTLKDVTLRNRIAIPPMCQYMAEDGMINDWHHVHLAGLARGGAGLLVVEATAVAPEGRI
TPGCAGIWSDAHAQAFVPVVQAIKAAGSVPGIQIAHAGRKASANRPWEGDDHIAADDTRGWETIAP
SAIAFGAHLPKVPREMTLDDIARVKQDFVDAARRARDAGFEWIELHFAHGYLGQSFFSEHSNKRTD
AYGGSFDNRSRFLLETLAAVREVWPENLPLTARFGVLEYDGRDEQTLEESIELARRFKAGGLDLLS
VSVGFTIPDTNIPWGPAFMGPIAERVRREAKLPVTSAWGFGTPQLAEAALQANQLDLVSVGRAHLA
DPHWAYFAAKELGVEKASWTLPAPYAHWLERYR
SEQ ID NO: 36: NADPH dehydrogenase, ‘FOYE-1’, ‘Ferrovum’ strain
JA12.
MSLLFSPYQLGSLSLANRLVIAPMCQYSAVDGIAQDWHLMHLGRLAISGAGLVIVEATGV
NPEGRITPFCLGLYNDEQEAALGRIVAFAREFGQAKMAIQLAHAGRKASTRRPWDPGSPY
SPEEGGWQTWAPSAIKFYEESLTPHPMSIEDLETVKQDFVNSAIRAERAGFKAIELHGAH
GYLIHQFLSPLSNQRQDQYGGSLENRMRYPLEILSAVKHALSAEMVVGMRISAVDWAPGG
LTIEESITFSQECEKRGAGFIHVSTGGLVAHQQIPVGPGYQVEHAQAIKQNVNIPTMAVG
LITHSAQAETILKSEQADMIAIARAALKNPHWPWTAALELGDKPFAPPQYQRAR
SEQ ID NO: 37: Oxidored_FMN domain-containing protein, ‘MgER’,
Meyerozyma guilliermondii.
MSVNINPLGETQVFQPIKLGKNTLSHRVFFPPTTRTRSLEDHTPSNLAYKYYDERSKFPG
TLIISEGTFPSAQAGLYEGVPGIWTERQTKTWKHIIDKIHENKSFASIQLWNLGRTGDPA
LLKKAGKPFLAPSAIYFDEESKKAAEKAGNPLRAMTEEEIKDMIYEQYTIAAKNALEAGF
DYIELHSAHGYLLHEFLEESSNKRTDKYGGSIENRARFVLELVDHMISIVGAERLGIRIS
PWATFQGMKSVHGEVHPLTTYSYLVNELEKRAQAGNRLAYISLVEPRVDGINSVEKKDQT
GNNDFVKDLWKGTILKAGNYTYDAPKFGQLLDDVSDGRTLVGFSRYFISNPDLISRLEKG
HQLAPYERETFYGRSDFGYNDYPKYGEKREDAEVAKKRVPEELVV
SEQ ID NO: 38: Oxidored_FMN domain-containing protein, ‘ClER’,
Clavispora (Candida) lusitaniae.
MVAVKPLKDTEIFKPTKVGNHELSNKIVYAPTTRMRAIADHTPSDLAYKYYDDRTKYPGS
LVITEATLMSPKTGLYDRVPGIYTDEHVAGWKKITDKIHANGSKVSMQLWPLGRVADPVA
TKKAGYPLVAPSLIYPSEEAKKAAEEAGNPIHVLTTEEVEDLVNDFVHAAKKAVAAGVDY
VEVHGAHGYLVDTFFQVSTNKRTDKYGGSIENRARFALEILDRLIEEIGAERVAIRISPW
AKFQGILAEEGEVNPVAQFGYFLSELENRARAGKRIAYVSIVEPRVSGVIDVAGEDIQGD
NSFVRSVWKGIVIKAGNYTYDAPEFKTLLQDVSDGKTLVAFARYFTSNPDLVQRLHDGAD
LTPYKRELFYAPSNWGYNTFTNAGETKTFSEEEESKRLPAPIDTKA
SEQ ID NO: 39: Tryptophan 2-Halogenase, ‘CmdE’, Chondromyces
crocatus.
MQLPSSTKILVVGGGPAGSTAATLLAREGFEVTLVEKAIFPRYHIGESLLISVQPIIDLL
GAREAVEAHGFQRKKGVLWEWGGERWLFDWKKLRYDYTFHVKREEFDEILLRNAQKNGVK
VFEGIDISRLEFDGERPVAAKWSKSSTGESGTIQFEFLLDASGRAGLMATQYLRSRMFMK
AFQNVATWGYWKGATIPEVEVEGPITVGSIPYGWIWGIPLRDQTMSVGLVIHQELFKEKR
ATQSVEEIYHEGLKASPLFQDVVLKGATLEPQIRTETDYSYISRTLAGPGFFLVGDSGAE
IDPLLSSGVHLAMHSALLAAASVKSIIAGEVDMASATEFYQRCYQGHFLRWALIVASFYE
VNARKETYFWTAQQLAHEELGVFNMSQADMKDVFATMVSGVVDLGDAQNAGRLQKGAERV
HQYLDDDGREEDVTALLQKSKQRIFEYLDRVKNRDSRAAMQRYKAGGTETFSMGLDADGA
VGGLYVTTEPRLGLLRKVVEERAEAATEAPAPAAPPPAVAEV
SEQ ID NO: 40: Tryptophan 5-Halogenase, ‘PyrH’, Streptomyces
rugosporus.
MIRSVVIVGGGTAGWMTASYLKAAFDDRIDVTLVESGNVRRIGVGEATFSTVRHFFDYLG
LDEREWLPRCAGGYKLGIRFENWSEPGEYFYHPFERLRVVDGFNMAEWWLAVGDRRTSFS
EACYLTHRLCEAKRAPRMLDGSLFASQVDESLGRSTLAEQRAQFPYAYHFDADEVARYLS
EYAIARGVRHVVDDVQHVGQDERGWISGVHTKQHGEISGDLFVDCTGFRGLLINQTLGGR
FQSFSDVLPNNRAVALRVPRENDEDMRPYTTATAMSAGWMWTIPLFKRDGNGYVYSDEFI
SPEEAERELRSTVAPGRDDLEANHIQMRIGRNERTWINNCVAVGLSAAFVEPLESTGIFF
IQHAIEQLVKHFPGERWDPVLISAYNERMAHMVDGVKEFLVLHYKGAQREDTPYWKAAKT
RAMPDGLARKLELSASHLLDEQTIYPYYHGFETYSWITMNLGLGIVPERPRPALLHMDPA
PALAEFERLRREGDELIAALPSCYEYLASIQ
SEQ ID NO: 41: Flavin-Dependent Tryptophan Halogenase, ‘RebH’,
Lentzea aerocolonigenes (Lechevalierla aerocolonigenes)
(Saccharothrix aerocolonigenes).
MSGKIDKILIVGGGTAGWMAASYLGKALQGTADITLLQAPDIPTLGVGEATIPNLQTAFF
DFLGIPEDEWMRECNASYKVAIKFINWRTAGEGTSEARELDGGPDHFYHSFGLLKYHEQI
PLSHYWFDRSYRGKTVEPFDYACYKEPVILDANRSPRRLDGSKVTNYAWHFDAHLVADFL
RRFATEKLGVRHVEDRVEHVQRDANGNIESVRTATGRVFDADLFVDCSGFRGLLINKAME
EPFLDMSDHLLNDSAVATQVPHDDDANGVEPFTSAIAMKSGWTWKIPMLGRFGTGYVYSS
RFATEDEAVREFCEMWHLDPETQPLNRIRFRVGRNRRAWVGNCVSIGTSSCFVEPLESTG
IYFVYAALYQLVKHFPDKSLNPVLTARFNREIETMFDDTRDFIQAHFYFSPRTDTPFWRA
NKELRLADGMQEKIDMYRAGMAINAPASDDAQLYYGNFEEEFRNFWNNSNYYCVLAGLGL
VPDAPSPRLAHMPQATESVDEVFGAVKDRQRNLLETLPSLHEFLRQQHGR
SEQ ID NO: 42: Flavin-Dependent Tryptophan Halogenase, ‘PrnA’,
Pseudomonas fluorescens.
MNKPIKNIVIVGGGTAGWMAASYLVRALQQQANITLIESAAIPRIGVGEATIPSLQKVFF
DFLGIPEREWMPQVNGAFKAAIKFVNWRKSPDPSRDDHFYHLFGNVPNCDGVPLTHYWLR
KREQGFQQPMEYACYPQPGALDGKLAPCLSDGTRQMSHAWHFDAHLVADFLKRWAVERGV
NRVVDEVVDVRLNNRGYISNLLTKEGRTLEADLFIDCSGMRGLLINQALKEPFIDMSDYL
LCDSAVASAVPNDDARDGVEPYTSSIAMNSGWTWKIPMLGRFGSGYVFSSHFTSRDQATA
DFLKLWGLSDNQPLNQIKFRVGRNKRAWVNNCVSIGLSSCFLEPLESTGIYFIYAALYQL
VKHFPDTSFDPRLSDAFNAEIVHMFDDCRDFVQAHYFTTSRDDTPFWLANRHDLRLSDAI
KEKVQRYKAGLPLTTTSFDDSTYYETFDYEFKNFWLNGNYYCIFAGLGMLPDRSLPLLQH
RPESIEKAEAMFASIRREAERLRTSLPTNYDYLRSLRDGDAGLSRGQRGPKLAAQESL
SEQ ID NO: 43: Thermophilic Tryptophan Halogenase, ‘Th-Hal’,
Streptomyces violaceusnige.
LNNVVIVGGGTAGWMTASYLKAAFGDRIDITLVESGHIGAVGVGEATFSDIRHFFEFLGLKEKDWM
PACNATYKLAVRFENWREKGHYFYHPFEQMRSVNGFPLTDWWLKQGPTDRFDKDCFVMASVIDAGL
SPRHQDGTLIDQPFDEGADEMQGLTMSEHQGKTQFPYAYQFEAALLAKYLTKYSVERGVKHIVDDV
REVSLDDRGWITGVRTGEHGDLTGDLFIDCTGFRGLLLNOALEEPFISYODTLPNDSAVALQVPMD
MERRGILPCTTATAQDAGWIWTIPLTGRVGTGYVYAKDYLSPEEAERTLREFVGPAAADVEANHIR
MRIGRSRNSWVKNCVAIGLSSGFVEPLESTGIFFTHHAIEQLVKNFPAADWNSMHRDLYNSAVSHV
MDGVREFLVLHYVAAKRNDTQYWRDTKTRKIPDSLAERIEKWKVQLPDSETVYPYYHGLPPYSYMC
ILLGMGGIELKPSPALALADGGAAQREFEQIRNKTQRLTEVLPKAYDYFTQ
SEQ ID NO: 44: Tryptophan 6-Halogenase, ‘SttH’, Streptomyces
toxytricini.
MNTRNPDKVVIVGGGTAGWMTASYLKKAFGERVSVTLVESGTIGTVGVGEATFSDIRHFF
EFLDLREEEWMPACNATYKLAVRFQDWQRPGHHFYHPFEQMRSVDGFPLTDWWLQNGPTD
RFDRDCFVMASLCDAGRSPRYLNGSLLQQEFDERAEEPAGLTMSEHQGKTQFPYAYHFEA
ALLAEFLSGYSKDRGVKHVVDEVLEVKLDDRGWISHVVTKEHGDIGGDLFVDCTGFRGVL
LNQALGVPFVSYQDTLPNDSAVALQVPLDMEARGIPPYTRATAKEAGWIWTIPLIGRIGT
GYVYAKDYCSPEEAERTLREFVGPEAADVEANHIRMRIGRSEQSWKNNCVAIGLSSGFVE
PLESTGIFFIHHAIEQLVKHFPAGDWHPQLRAGYNSAVANVMDGVREFLVLHYLGAARND
TRYWKDTKTRAVPDALAERIERWKVQLPDSENVFPYYHGLPPYSYMAILLGTGAIGLRPS
PALALADPAAAEKEFTAIRDRARFLVDTLPSQYEYFAAMGQRV
SEQ ID NO: 45: KtzQ, ‘KtzQ’, Kutzneria sp. 744.
MDDNRIRSILVLGGGTAGWMSACYLSKALGPGVEVTVLEAPSISRIRVGEATIPNLHKVF
FDFLGIAEDEWMRECNASYKAAVRFVNWRTPGDGQATPRRRPDGRPDHFDHLFGQLPEHE
NLPLSQYWAHRRLNGLTDEPFDRSCYVQPELLDRKLSPRLMDGTKLASYAWHFDADLVAD
FLCRFAVQKLNVTHVQDVFTHADLDQRGHITAVNTESGRTLAADLFIDCSGFRSVLMGKV
MQEPFLDMSKHLLNDRAVALMLPHDDEKVGIEPYTSSLAMRSGWSWKIPLLGRFGSGYVY
SSQFTSQDEAAEELCRMWDVDPAEQTFNNVRFRVGRSRRAWVRNCVAIGVSAMFVEPLES
TGLYFSYASLYQLVKHFPDKRFRPILADRFNREVATMYDDTRDFLQAHFSLSPRDDSEFW
RACKELPFADGFAEKVEMYRAGLPVELPVTIDDGHYYGNFEAEFRNFWTNSNYYCIFAGL
GFLPEHPLPVLEFRPEAVDRAEPVFAAVRRRTEELVATAPTMQAYLRRLHQGT
SEQ ID NO: 46: Monodechloroaminopyrrolnitrin halogenase, ‘PrnC’,
Pseudomonas fluorescens.
MTQKSPANEHDSNHFDVIILGSGMSGTQMGAILAKQQFRVLIIEESSHPRFTIGESSIPE
TSLMNRIIADRYGIPELDHITSFYSTQRYVASSTGIKRNFGFVFHKPGQEHDPKEFTQCV
IPELPWGPESHYYRQDVDAYLLQAAIKYGCKVHQKTTVTEYHADKDGVAVTTAQGERFTG
RYMIDCGGPRAPLATKFKLREEPCRFKTHSRSLYTHMLGVKPFDDIFKVKGQRWRWHEGT
LHHMFEGGWLWVIPFNNHPRSTNNLVSVGLQLDPRVYPKTDISAQQEFDEFLARFPSIGA
QFRDAVPVRDWVKTDRLQFSSNACVGDRYCLMLHANGFIDPLFSRGLENTAVTIHALAAR
LIKALRDDDFSPERFEYIERLQQKLLDHNDDFVSCCYTAFSDFRLWDAFHRLWAVGTILG
QFRLVQAHARFRASRNEGDLDHLDNDPPYLGYLCADMEEYYQLFNDAKAEVEAVSAGRKP
ADEAAARIHALIDERDFAKPMFGFGYCITGDKPQLNNSKYSLLPAMRLMYWTQTRAPAEV
KKYFDYNPMFALLKAYITTRIGLALKK
SEQ ID NO: 47: FADH2-dependent halogenase, ‘PltA’, Pseudomonas
protegens Pf-5.
GPHMSDHDYDVVIIGGGPAGSTMASYLAKAGVKCAVFEKELFEREHVGESLVPATTPVLLEIGVME
KIEKANFPKKFGAAWTSADSGPEDKMGFQGLDHDFRSAEILFNERKQEGVDRDFTFHVDRGKFDRI
LLEHAGSLGAKVFQGVEIADVEFLSPGNVIVNAKLGKRSVEIKAKMVVDASGRNVLLGRRLGLREK
DPVFNQFAIHSWFDNFDRKSATQSPDKVDYIFIHFLPMTNTWVWQIPITETITSVGVVTQKQNYTN
SDLTYEEFFWEAVKTRENLHDALKASEQVRPFKKEADYSYGMKEVCGDSFVLIGDAARFVDPIFSS
GVSVALNSARIASGDIIEAVKNNDFSKSSFTHYEGMIRNGIKNWYEFITLYYRLNILFTAFVQDPR
YRLDILQLLQGDVYSGKRLEVLDKMREIIAAVESDPEHLWHKYLGDMQVPTAKPAFEND
SEQ ID NO: 48: Halogenase, ‘PltM’, Pseudomonas fluorescens (strain
ATCC BAA-477/NRRL B-23932/Pf-5).
MNQYDVIIIGSGIAGALTGAVLAKSGLNVLILDSAQHPRFSVGEAATPESGFLLRLLSKR
FDIPEIAYLSHPDKIIQHVGSSACGIKLGFSFAWHQENAPSSPDHLVAPPLKVPEAHLFR
QDIDYFALMIALKHGAESRQNIKIESISLNDDGVEVALSNAAPVKAAFIIDAAAQGSPLS
RQLGLRTTEGLATDTCSFFTHMLNVKSYEDALAPLSRTRSPIELFKSTLHHIFEEGWLWV
IPFNNHPQGTNQLCSIGFQFNNAKYRPTEAPEIEFRKLLKKYPAIGEHFKDAVNAREWIY
APRINYRSVQNVGDRFCLLPQATGFIDPLFSRGLITTFESILRLAPKVLDAARSNRWQRE
QFIEVERHCLNAVATNDQLVSCSYEAFSDFHLNNVWHRVWLSGSNLGSAFLQKLLHDLEH
SGDARQFDAALEAVRFPGCLSLDSPAYESLFRQSCQVMQQAREQARPVAETANALHELIK
EHEAELLPLGYSRISNRFILKV
SEQ ID NO: 49: Flavin-Dependent Halogenase, ‘Clz5’, Streptomyces
sp. CNH-287.
MSEVDFDIGIIGGGPAGSAIASYLAKAGLNCVIFEGDIFPREHVGESLIPATTPVLDDIG
FLPQMETAGFPKKYGAAWTSASDQNIPTMGFEGMSHGFRAAEIEFHEREQLGVTQDYTYH
VDRAKFDLMLLQHAASLGAKVYQGVRVRRVDFSETNPRIIFPIGKTETSVRVRMVVDASG
RNTFLGRQLKLKVSDPVFNQYAVHTWFDNLDRTALSVNKEQADYIFIHFLPVTDTWVWQI
PITDTITSIGVVTQKEQFKASKEDLDGFFWECVGSRPELKEALEKSDQVRPFKTEGDYSY
AMKQIVGDQWATIGDAARFVDPIFSSGVSVALNSARLISGEIIAAAEQGDFRKEMFSNYE
GKIRRAVSNWYEFISIYYRLNILFTAFVQDPRYRLDVLKMLQGDVYDDEEPKALAAMREI
TKQVEENPDHLWHKHLGSLRAPSAAPMF
SEQ ID NO: 50: Pyrrole Halogenase, ‘Bmp2’, Pseudoalteromonas
piscicida.
MNGFTHYDVVIIGSGPAGSLCGIECRKKGMSVLCIEKEQFPRFHIGESLTGNAGQIIRDL
GLADAMDAAGFPDKPGVNVIGSLSKNEFFIPILAPTWQVRRSDFDNMIKQKAVEHGVEYQ
LGMVTDVIREGDKVVGAVYKADGIEHQVRSKVLVDASGQNTFLSRKGIAGKRQIEFFSQQ
IASFAHYKGVERDLPPFSTNTTILYSKQYHWSWIIPISPDTDSLGVVIPKDLYYKECKNP
DDAIEWGMEHISPELKRRFKNAERQGESQSMADFSYRIEPFVGDGWLCIGDAHRFLDPIF
SYGVSFAMKEGIRAAEAIEQVVNGQDWKAPFYAYRDWSNGGQQIAADLIRYFWIYPIFFG
YQMQNPDLRDEVIRLLGGCCFDCKDWKAPAIFRNAIEEYDRKQMAS
SEQ ID NO: 51: Non-Heme Halogenase, ‘Rdc2’, Metacordyceps
chlamydosporia (Pochonia chlamydosporia).
MSVPKSCTILVAGGGPAGSYAAAALAREGNDVVLLEADQHPRYHIGESMLPSLRPLLRFI
DLEDKFDAHGFQKKLGAAFKLTSKREGSHGPRGYSWNVVRSESDEILFNHARESGAQAFQ
GIKINAIEFEPYEEEYPNGEKVANPGKPTSAKWSSKDGSSGDIAFKYLVDATGRIGIMST
KYLKNRHYNEGLKNLAIWGYYKNNIPWAQGTPRENQPFFEGMRDGAGWCWTIPLHNGTVS
VGAVMRKDLFFEKKKSLGENATNTQIMAECMKLCPTIGELLAPAELVSDIKQATDYSYSA
TAYAGPNFRIVGDAGCFIDPFFSSGHHLAVAGALAAAVSINASIKGDCTEYEASRWHAKK
VDEGYTLFLLVVMAALKQIRMQENPVLSDVDEDGFDRAFQFLRPEAVKKFTKEDVAQTID
FAVHALNNMAELDMDIPEHMINGDKEGENGVTNGNNGAAKTAGLASNMEKLTNDEEKVLN
GLRILGKAAPGGTLADFEGTAIDGLMPRLEHGKLGLNKV
SEQ ID NO: 52: Tryptophan 6-Halogenase, ‘BorH’, uncultured
bacteria.
MDNRINRIVILGGGTAGWMTASYLAKALGDTVTITLLEAPAIGRIGVGEATVPNLQRVFF
DFLGLREEEWMPECNAAFKTAVKFINWRTPGPGEAKARTIDGRPDHFYHPFGLLPEHGQV
PLSHYWAYNRAAGTTDEPFDYACFAETAAMDAVRAPKWLDGRPATRYAWHFDAHLVAEFL
RRHATERLNVEHVQGEMQQVLRDERGFITALRTVEGRDLEGDLFIDCSGFRGLLINKAME
EPFIDMNDQLLCNRAVATAIKHDDDAHGVEPYTSAIAMRSGWSWKIPMLGRFGTGYVYSS
RFAEKDEATLDFCRMWGLDPENTPLNQVAFRVGRNRRAWVKNCVSIGLASCFLEPLESTG
IYFITAAIYQLTQHFPDRTFALALSDAFNHEIEAMFDDTRDFIQAHFYVSPRTDTPFWKA
NKDLHLPEQMREKIAMYKAGLPINAPVTDESTYYGRFEAEFRNFWTNGSYYCIFAGLGLR
PDNPLPMLRHRPEQVREAQALFAGVKDKQRELVETLPSNLEFLRSLHGK
SEQ ID NO: 53: Styrene Monooxygenase,‘StyA’, Pseudomonas sp.
MKKRIGIVGAGTAGLHLGLELRQHDVDVTVYTDRKPDEYSGLRLLNTVAHNAVTVQREVA
LDVNEWPSEEFGYFGHYYYVGGPQPMRFYGDLKAPSRAVDYRLYQPMLMRALEARGGKFC
YDAVSAEDLEGLSEQYDLLVVCTGKYALGKVFEKQSENSPFEKPQRALCVGLFKGIKEAP
IRAVTMSFSPGHGELIEIPTLSFNGMSTALVLENHIGSDLEVLAHTKYDDDPRAFLDLML
EKLGKHHPSVAERIDPAEFDLANSSLDILQGGVVPAFRDGHATLNNGKTIIGLGDIQATV
DPVLGQGANMASYAAWILGEEILAHSVYDLRFSEHLERRRQDRVLCATRWTNFTLSALSA
LPPEFLAFLQILSQSREMADEFTDNFNYPERQWDRFSSPERIGQWCSQFAPTIAA
SEQ ID NO: 54: 4-Nitrophenol 2-Monooxygenase Oxygenase Component,
‘PheA1’, Rhodococcus erythropolis (Arthrobacter picolinophilus).
MTTTEIPPTGVDPADSGAPQVNPAADSEANRTKNFATRPMTGDEYISSLQDGREIWLHGD
RVKDVTTHPAFRNPIRMTARLYDALHTGEHVDALTVPTDTGNGGVTMPFFRTPTSSADLL
KERDAIATWARMTYGWMGRSPDYKASFLGTLHANKELYAPFQDNAERWYRESQEKVLYWN
HAIINPPVDRQLPPDEVGDVFMKVEKETDAGLIVSGAKVVATGSAITNYNFIAHYGLPIK
KKQFALICTVPMDAPGVKLICRTSYTEHAAVMGSPFDYPLSSRMDENDTIFVFDKVLVPW
ENVFMYGDVDRINAFFPQSGFLPRFTFQGCTRLAVKLDFIAGLLMKALEATGAGGFRGVQ
TRVGEVIGWRNLFWSLTESMARDPEQWVGDSVIPKLEYGLTYRMFMIQGYPRIKEIIEQD
VASGLIYLPSSSLDFKSPDVRPYLDKYVRGSDGITAVDRVKVMKALWDSIGTEFGGRHEL
YERNYSGNHENVKAELLFAAQNRGHASSMKGLAEQCLSEYDLDGWTVPDLIGNDDVSFFK
NR
SEQ ID NO: 55: 4-Hydroxyphenylacetate 3-Monooxygenase Oxygenase
Component, ‘HpaB’, Klebsiella oxytoca.
MKPENFRADTKRPLTGEEYLKSLQDGREIYIYGERVKDVTTHPAFRNAAASVAQLYDALH
NPELQNTLCWGTDTGSGGYTHKFFRVAKSADDLRQQRDAIAEWSRLSYGWMGRTPDYKAA
FGGGLGANPGFYGQFEQNARDWYTRIQETGLYFNHAIVNPPIDRHKPADEVKDVYIKLEK
ETDAGIIVSGAKVVATNSALTHYNMIGFGSAQVMGENPDFALMFVAPMDAEGDKLISRAS
YELVAGATGSPYDYPLSSRFDENDAILVMDNVLIPWENVLIYRDFDRCRRWTMEGGFARM
YPLQACVRLAVKLDFITALLKRSLECTGTLEFRGVQAELGEVVAWRNMFWALSDSMCAEA
TPWVNGAYLPDHAALQTYRVMAPMPYAKIKNIIERSVTSGLIYLPSSARDLNNPQINDTL
AKYVRGSNGMDHVERIKILKLMWDAIGSEFGGCHELYEINYSGSQDEIRLQCLRQAQSSG
NMDKMMAMVDRCLSEYDQNGWTVPHLHNNTDINMLDKLLK
SEQ ID NO: 56: Chlorophenol Monooxygenase, ‘HadA’, Ralstonia
pickettii (Burkholderia pickettii).
MIRTGTQYLESLNDGRNVWVGNEKIDNVATHPKTRDYAQRHADFYDLHHRPDLQDVMTYI
DEGGQRRAMQWFGHRDKEQLRRKRKYHETVMREMAGASFPRTPDVNNYVLTTYIDDPAPW
ETQSIGDDGHIKAGKIVDFIRYAREHDLNCAPQFVDPQMDRSNPDAQERSPGLRVVEKNE
KGIVVNGVKAIGTGVAFADWIHIGVFFRPGIPGDQVIFAATPVNTPGVTIVCRESLVKDD
KVEHPLAAQGDELDGMTVFENVFIPWSHVFHIGNPNHAKLYPQRVFDWLHYHALIRQMVR
AELVAGLAVLITEHIGTNKIPAVQTRVAKLIGFHQAMLAHLIASEELGFHTPGGHYKPNI
LIYDFGRALYLENFSQMIYELVDLSGRSALIFASEDQWNDDKLNGWFERMNNGPVGRPHD
RVKIGRVIRDLFLTDWGSRLFVFENFNGTPLQGIRMLTMQRAEFSGSGPYGKLARQVCGI
DSAVTDDTEYRKTADYAKALDAARHQEEVALAGAMAI
SEQ ID NO: 57: Tetrachlorobenzoquinone Reductase, ‘PcpD’,
Sphingobium chlorophenolicum.
MTNPVSTIDMTVTQITRVAKDINSYELRPEPGVILPEFTAGAHIGVSLPNGIQRSYSLVN
PQGERDRYVITVNLDRNSRGGSRYLHEQLRVGQRLSIVPPANNFALVETAPHSVLFAGGI
GITPIWSMIQRLRELGSTWELHYACRGKDFVAYRQELEQAAAEAGARFHLHLDEEADGKF
LDLAGPVAQAGQDSIFYCCGPEAMLQAYKAATADLPSERVRFEHFGAALTGEPADDVFTV
VLARRSGQEFTVEPGMTILETLLQNGISRNYSCTQGVCGTCETKVLEGEPDHRDWVLSDE
KKASNSTMLICCSLSKSPRLVLDI
SEQ ID NO: 58: 2-Methyl-6-ethyl-4-monooxygenase Oxygenase
Component,‘MeaX’, Sphingobium baderi.
MTQAIDAGERVVSLDLETHGPEALIKRAAELVPLLRENAVRCHNERRIPSENLSAMRQAG
LLRMSRPRRYGGHEAMVATKTAVFGELARGCGSTAWVTTLYEDAAFLISLFPDEVQDEVF
ADQDTLITATLIPAGRAQPQGEGFLVNGRWPFNTGCLDATYVVEPAVVELSRGAPEVCLF
LMPYSELVIEDDWSPVGLRGTGSNSVRAKDVYVRPERMLRLADVMRGIYGTKLNKGPLYR
VPPIPYIVSSAGGTFPGLAQSAFELVTERLVGRPITYTLYTDRAQAPVTHLQLGEAALRI
KAANHLMTEVADRLDRRALNNEALTPDEGPMIWGIIGYTSRIYAEVIESLRQMSGASALS
ETSAIQAVARNAQALATHAMLIPTTGIEHYGRAICGLPPNTPFLSS
SEQ ID NO: 59: Alkanesulfonate Monooxygenase, ‘SsuD’, Escherichia
coli (strain K12).
MSLNMFWFLPTHGDGHYLGTEEGSRPVDHGYLQQIAQAADRLGYTGVLIPTGRSCEDAWL
VAASMIPVTQRLKFLVALRPSVTSPTVAARQAATLDRLSNGRALFNLVTGSDPQELAGDG
VFLDHSERYEASAEFTQVWRRLLQRETVDFNGKHIHVRGAKLLFPAIQQPYPPLYFGGSS
DVAQELAAEQVDLYLTWGEPPELVKEKIEQVRAKAAAHGRKIRFGIRLHVIVRETNDEAW
QAAERLISHLDDETIAKAQAAFARTDSVGQQRMAALHNGKRDNLEISPNLWAGVGLVRGG
AGTALVGDGPTVAARINEYAALGIDSFVLSGYPHLEEAYRVGELLFPLLDVAIPEIPQPQ
PLNPQGEAVANDFIPRKVAQS
SEQ ID NO: 60: p-Hydroxyphenylacetate 3-Hydroxylase, Oxygenase
Component, ‘C2-HpaH’, Acinetobacter baumannii.
MENTVLNLDSDVIHACEAIFQPIRLVYTHAQTPDVSGVSMLEKIQQILPQIAKNAESAEQ
LRRVPDENIKLLKEIGLHRAFQPKVYGGLEMSLPDFANCIVTLAGACAGTAWAFSLLCTH
SHQIAMFSKQLQDEIWLKDPDATASSSIAPFGKVEEVEGGIILNGDYGWSSGCDHAEYAI
VGFNRFDADGNKIYSFGVIPRSDYEIVDNWYAQAIKSSGSKMLKLVNVFIPEYRISKAKD
MMEGKSAGFGLYPDSKIFYTPYRPYFASGFSAVSLGIAERMIEAFKEKQRNRVRAYTGAN
VGLATPALMRIAESTHQVAAARALLEKTWEDHRIHGLNHQYPNKETLAFWRTNQAYAVKM
CIEAVDRLMAAAGATSFMDNSELQRLFRDAHMTGAHAYTDYDVCAQILGRELMGMEPDPT
MV
SEQ ID NO: 61: FADH(2)-Dependent Monooxygenase, ‘TftD’,
Burkholderia cepacia (Pseudomonas cepacia).
MRTGKQYLESLNDGRVVWVGNEKIDNVATHPLTRDYAERVAQFYDLHHRPDLQDVLTFVD
ADGVRRSRQWQDPKDAAGLRVKRKYHETILREIAAGSYGRLPDAHNYTFTTYADDPEVWE
KQSIGAEGRNLTQNIHNFLKLLREKDLNCPLNFVDPQTDRSSDAAQARSPNLRIVEKTDD
GIIVNGVKAVGTGIAFGDYMHIGCLYRPGIPGEQVIFAAIPTNTPGVTVFCRESTVKNDP
AEHPLASQGDELDSTTVFDNVFIPWEQVFHIGNPEHAKLYPQRIFDWVHYHILIRQVLRA
ELIVGLAILITEHIGTSKLPTVSARVAKLVAFHLAMQAHLIASEETGFHTKGGRYKPNPL
IYDFGRAHFLQNQMSVMYELLDLAGRSSLMIPSEGQWDDSQSGQWFVKLNNGPKGNPRER
VQIGRVIRDLYLTDWGGRQFMFENFNGTPLFAVFAATMTRDDMSAAGTYGKFASQVCGIE
FGGAEPTAYAATADYARALDRGLAPEPAAAESATS
SEQ ID NO: 62: 4-Nitrophenol 2-Monooxygenase, Oxygenase Component,
‘NphA1’, Rhodococcus sp.
MTTSAFVDDRVGVPNDVRPMTGDEYLESLRDGREVYFRGERVDDVTTHPAFRNSARSVAR
MYDALHQPEQEGVLAVPTDTGNGGFTHPFFRTARSADDLVLSRDAIVAWQREVYGWLGRS
PDYRASFLGTLGANADFYGPYRDNALRWYRHAQERMLYLNHAIVNPPIDRDRPADETADV
CVHVVEETDAGLIVSGAKVVATGSAITNANFIAHYGLLRKKEYGLIFTVPMDSPGLKLFC
RTSYEMNAAVMGTPFDYPLSSRFDENDAIMVFDRVLVPWENVFAYDTDTANGFVMRSGFL
SRFMFHGCARLAVKLDFIAGCVMKGVEMTGSAGFRGVQMQIGEILNWRDMFWGLSDAMAK
SPEQWVNGAVQPNLNYGLAYRTFMGVGYPRVKEIIQQVLGSGLIYLNSHASDWANPAMRP
YLDQYVRGSNGVAAIDRVQLLKLLWDAVGTEFGGRHELYERNYGGDHEAVRFQTLFAYQA
TGQDLALKGFAEQCMSEYDVDGWTRPDLIGNDDLRIVRG
SEQ ID NO: 63: Putative dehydrogenase/oxygenase subunit,
‘VpStyA1’, Variovorax paradoxus (strain EPS).
MKRIAIVGAGQSGLQLGLGLLAAGYEVTMFSNRTGEDIRRGKVMSSQCMFDTSLQIERDL
GLDHWASDCPTVDGIGLAVPHPEQKGAKVIDWAARLNASAQSVDQRLKIPAWMDEFQKKG
GELVFKDAGIDELEACTQSHDLTLVASGKGEISKLFERDAHKSPYDKPQRALALTYVKGM
APREPFSAVCFNLIPGVGEYFVFPALTTTGPCEIMVFEGVPGGPMDCWADVKTPEEHLAR
SKWILDTFTPWEAERCKDIELTDDNGILAGRFAPTVRKPVATLPSGRKVLGLADVVVLND
PITGQGSNNAAKCADTYLKSILARGDGAADAAWMQQTFDRYWFGYAQWVTQWTNMLLAPP
PPHVLNLLGSAGAVPPLASAFANGFDDPRTFFPWFADAAESERYIATCAAVA
SEQ ID NO: 64: Oxygenase, ‘RoIndA1’ {from styA1 gene}, Rhodococcus
opacus (Nocardia opaca).
MRKITIVGAGQAGLQLAIGLVDAGYDVTVVSNRTPEQIREGKVMSSQCMFGGALARERAV
GLDLWSDECPSVEGISFTVADNGNQAFSWASRLDLPAQSVDQRVKMPAWLKEFEARGGTL
VLQDAGIGDLEGFAVDSDLVILAAGKGEIAKMFERDATRSVFDRPQRALALTYVNGMVPR
EEHSAVAFNMIPGVGEYFAFPALTTSGPCEIMVMEGIPGGPMDCWADVTTPEEHLAKSKW
VVETFVPWEAERCADITLTDDNGILAGRFPPTVRKPIGVLPSGANILGLADVVVLNDPLT
GQGSNNASKCAASYLASILEHGGRPFDAEFMTDTFERYWDYAQYVASWTNALLSPPPEHV
LKLLGAAATEPRIARRFANGFDDPRDFYHWFMTPEAAAQYLTDVAN
SEQ ID NO: 65: Smoa_sbd domain-containing protein, ‘AbIndA’,
Acinetobacter baylyi (strain ATCC 33305/BD413/ADP1).
MMRRIAIVGAGQSGLQLGLSLLDTGYDVTIVTNRTADQIRQGKVMSSQCMFHTALQTERD
VGLNFWEEQCPAVEGIGFTLVSPETGKPAFSWSARLERYAQSVDQRVKMPYWIEEFERRG
GKLIIQDVGIDELEQLTTEYELVLLAAGKGEVVKQFVRDDERSTFDKPQRALALTYVTGM
KPMSPYSRVTFNVIPGVGEYFCFPALTVTGPCEIMVFEGIPGGPMDCWQDAKTPEQHLQM
SKDILNTYLPWEAERCENIEITDAGGYLAGRFPPSVRKPILTLPSGRQVFGMADALVVND
PITGQGSNNAAKCSKIYFDAILAHDTQSFTPEWMQQTFERYWSYAEKVVAWTNSLLVPPQ
PQMIDVLAAASQNQAIASTIANNFDDPRNFSPWWFDAEQAQHFIESKSCQKVA
SEQ ID NO: 66: 2,5-Diketocamphane 1,2-Monooxygenase 1, ‘CamP’,
Pseudomonas putida (Arthrobacter siderocapsulatus).
MKCGFFHTPYNLPTRTARQMFDWSLKLAQVCDEAGFADFMIGEHSTLAWENIPCPEIIIG
AAAPLTKNIRFAPMAHLLPYHNPATLAIQIGWLSQILEGRYFLGVAPGGHHTDAILHGFE
GIGPLQEQMFESLELMEKIWAREPFMEKGKFFQAGFPGPDTMPEYDVEIADNSPWGGRES
MEVAVTGLTKNSSSLKWAGERNYSPISFFGGHEVMRSHYDTWAAAMQSKGFTPERSRFRV
TRDIFIADTDAEAKKRAKASGLGKSWEHYLFPIYKKFNLFPGIIADAGLDIDPSQVDMDF
LAEHVWLCGSPETVKGKIERMMERSGGCGQIVVCSHDNIDNPEPYFESLQRLASEVLPKV
RMG
SEQ ID NO: 67: 3,6-Diketocamphane 1,6-Monooxygenase, ‘CamE36’,
Pseudomonas putida (Arthrobacter siderocapsulatus).
MAMETGLIFHPYMRPGRSARQTFDWGIKSAVQADSVGIDSMMISEHASQIWENIPNPELL
IAAAALQTKNIKFAPMAHLLPHQHPAKLATMIGWLSQILEGRYFLGIGAGAYPQASYMHG
IRNAGQSNTATGGEETKNLNDMVRESLFIMEKIWKREPFFHEGKYWDAGYPEELEGEEGD
EQHKLADFSPWGGKAPEIAVTGFSYNSPSMRLAGERNFKPVSIFSGLDALKRHWEVYSEA
AIEAGHTPDRSRHAVSHTVFCADTDKEAKRLVMEGPIGYCFERYLIPIWRRFGMMDGYAK
DAGIDPVDADLEFLVDNVFLVGSPDTVTEKINALFEATGGWGTLQVEAHDYYDDPAPWFQ
SLELISKEVAPKILLPKR
SEQ ID NO: 68: Alkanal monooxygenase, alpha chain, ‘LuxA’, Vibrio
harveyi (Beneckea harveyi).
MKFGNFLLTYQPPELSQTEVMKRLVNLGKASEGCGFDTVWLLEHHFTEFGLLGNPYVAAA
HLLGATETLNVGTAAIVLPTAHPVRQAEDVNLLDQMSKGRERFGICRGLYDKDFRVFGTD
MDNSRALMDCWYDLMKEGFNEGYIAADNEHIKFPKIQLNPSAYTQGGAPVYVVAESASTT
EWAAERGLPMILSWIINTHEKKAQLDLYNEVATEHGYDVTKIDHCLSYITSVDHDSNRAK
DICRNFLGHWYDSYVNATKIFDDSDQTKGYDFNKGQWRDFVLKGHKDTNRRIDYSYEINP
VGTPEECIAIIQQDIDATGIDNICCGFEANGSEEEIIASMKLFQSDVMPYLKEKQ
SEQ ID NO: 69: Alkanal monooxygenase, beta chain, ‘LuxB’, Vibrio
harveyi (Beneckea harveyi).
MKFGLFFLNFMNSKRSSDQVIEEMLDTAHYVDQLKFDTLAVYENHFSNNGVVGAPLTVAG
FLLGMTKNAKVASLNHVITTHHPVRVAEEACLLDQMSEGRFAFGFSDCEKSADMRFFNRP
TDSQFQLFSECHKIINDAFTTGYCHPNNDFYSFPKISVNPHAFTEGGPAQFVNATSKEVV
EWAAKLGLPLVFRWDDSNAQRKEYAGLYHEVAQAHGVDVSQVRHKLTLLVNQNVDGEAAR
AEARVYLEEFVRESYSNTDFEQKMGELLSENAIGTYEESTQAARVAIECCGAADLLMSFE
SMEDKAQQRAVIDVVNANIVKYHS
SEQ ID NO: 70: Alkanal monooxygenase, alpha chain,‘LuxA’,
Photorhabdus luminescens (Xenorhabdus luminescens).
MKFGNFLLTYQPPELSQTEVMKRLVNLGKASEGCGFDTVWLLEHHFTEFGLLGNPYVAAA
HLLGATETLNVGTAAIVLPTAHPVRQAEDVNLLDQMSKGRFRFGICRGLYDKDFRVFGTD
MDNSRALMDCWYDLMKEGFNEGYIAADNEHIKFPKIQLNPSAYTQGGAPVYVVAESASTT
EWAAERGLPMILSWIINTHEKKAQLDLYNEVATEHGYDVTKIDHCLSYITSVDHDSNRAK
DICRNFLGHWYDSYVNATKIFDDSDQTKGYDFNKGQWRDFVLKGHKDTNRRIDYSYEINP
VGTPEECIAIIQQDIDATGIDNICCGFEANGSEEEIIASMKLFQSDVMPYLKEKQ
SEQ ID NO: 71: Alkanal monooxygenase, beta chain, ‘LuxB’,
Photorhabdus luminescens (Xenorhabdus luminescens).
MKFGLFFLNFINSTTVQEQSIVRMQEITEYVDKLNFEQILVYENHFSDNGVVGAPLTVSG
FLLGLTEKIKIGSLNHIITTHHPVAIAEEACLLDQLSEGRFILGFSDCEKKDEMHFFNRP
VEYQQQLFEECYEIINDALTTGYCNPDNDFYSFPKISVNPHAYTPGGPRKYVTATSHHIV
EWAAKKGIPLIFKWDDSNDVRYEYAERYKAVADKYDVDLSEIDHQLMILVNYNEDSNKAK
QETRAFISDYVLEMHPNENFENKLEEIIAENAVGNYTECITAAKLAIEKCGAKSVLLSFE
PMNDLMSQKNVINIVDDNIKKYHMEYT
SEQ ID NO: 72: Alkane Monooxygenase, ‘LadA’, Geobacillus
thermodenitrificans.
MTKKIHINAFEMNCVGHIAHGLWRHPENQRHRYTDLNYWTELAQLLEKGKFDALFLADVVGIYDVY
RQSRDTAVREAVQIPVNDPLMLISAMAYVTKHLAFAVTFSTTYEHPYGHARRMSTLDHLTKGRIAW
NVVTSHLPSADKNFGIKKILEHDERYDLADEYLEVCYKLWEGSWEDNAVIRDIENNIYTDPSKVHE
INHSGKYFEVPGPHLCEPSPQRTPVIYQAGMSERGREFAAKHAECVFLGGKDVETLKFFVDDIRKR
AKKYGRNPDHIKMFAGICVIVGKTHDEAMEKLNSFQKYWSLEGHLAHYGGGTGYDLSKYSSNDYIG
SISVGEIINNMSKLDGKWFKLSVGTPKKVADEMQYLVEEAGIDGFNLVQYVSPGTFVDFIELVVPE
LQKRGLYRVDYEEGTYREKLFGKGNYRLPDDHIAARYRNISSNV
SEQ ID NO: 73: EDTA Monooxygenase, ‘EmoA’, Chelativorans
multitrophicus.
PAVAANYGAAIATHDNRYERAEEFLEVVHGLWNSWKFPWDEAIGPNPNPFGEVMPINHEG
KYFKVAGPLNVPLPPYGPPVVVQAGGSDQGKRLASRFGEIIYAFLGSKPAGRRFVAEARA
AARAQGRPEGSTLVLPSFVPLIGSTEAEVKRLVAEYEAGLDPAEQRIEALSKQLGIDLER
INVDQVLQEKDFNLPKESATPIGILKSMVDVALDEKLSLRQLA
SEQ ID NO: 74: Isobutylamine N-hydroxylase, ‘IBAH’, Streptomyces
viridifaciens.
MRSLDAARDTCERLHPGLIKALEELPLLEREAEGSPVLDIFRAHGGAGLLVPSAYGGHGA
DALDAVRVTRALGACSPSLAAAATMHNFTAAMLFALTDRVIPPTDEQKKLLARVAPEGML
LASGWAEGRTQQDILNPSVKATPVDDGFILNGSKKPCSLSRSMDILTASVILPDETGQQS
LAVPLIMADSPGISVHPFWESPVLAGSQSNEVRLKDVHVPEKLIIRGTPDDPGRLDDLQT
ATFVWFELLITSAYVGAASALTELVMERDRGSVTDRAALGIQLESAVGLTEGVARAVRDG
VFGEEAVAAALTARFAVQKTLAAISDQAIELLGGIAFIKSPELAYLSSALHPLAFHPPGR
TSSSPHLVEYFSGGPLEI
SEQ ID NO: 75: ActVA 6 Protein, ‘ActVA-Orf6’, Streptomyces
coelicolor.
MAEVNDPRVGFVAVVTFPVDGPATQHKLVELATGGVQEWIREVPGFLSATYHASTDGTAV
VNYAQWESEQAYRVNFGADPRSAELREALSSLPGLMGPPKAVFMTPRGAILPS
SEQ ID NO: 76: Pyrimidine Monooxygenase, ‘RutA’, Escherichia coli
(strain K12).
MQDAAPRLTFTLRDEERLMMKIGVFVPIGNNGWLISTHAPQYMPTFELNKAIVQKAEHYH
FDFALSMIKLRGFGGKTEFWDHNLESFTLMAGLAAVTSRIQIYATAATLTLPPAIVARMA
ATIDSISGGRFGVNLVTGWQKPEYEQMGIWPGDDYFSRRYDYLTEYVQVLRDLWGTGKSD
FKGDFFTMNDCRVSPQPSVPMKVICAGQSDAGMAFSARYADFNFCFGKGVNTPTAFAPTA
ARMKQAAEQTGRDVGSYVLFMVIADETDDAARAKWEHYKAGADEEALSWLTEQSQKDTRS
GTDTNVRQMADPTSAVNINMGTLVGSYASVARMLDEVASVPGAEGVLLTFDDFLSGIETF
GERIQPLMQCRAHLPALTQEVA
SEQ ID NO: 77: p-Hydroxyphenylacetate 2-Hydroxylase Reductase
Component, ‘C1-HpaH’, Acinetobacter baumannii.
MNQLNTAIVEKEVIDPMAFRRALGNFATGVTIMTAQTSSGERVGVTANSFNSVSLDPALV
LWSIDKKSSSYRIFEEATHFGVNILSAAQIELSNRFARRSEDKFANIEFDLGVGNIPLFK
NCSAAFECERYNIVEGGDHWIIIGRVVKFHDHGRSPLLYHQGAYSAVLPHPSLNMKSETA
EGVFPGRLYDNMYYLLTQAVRAYQNDYQPKQLASGFRTSEARLLLVLESKTASSKCDLQR
EVAMPIREIEEATKILSEKGLLIDNGQHYELTEQGNACAHMLYKIAESHQEEVFAKYTVD
ERKLFKNMLKDLIGI
SEQ ID NO: 78: FMN_red Domain-Containing Protein, ‘YdhA’, Bacillus
subtilis subsp. natto (strain BEST195).
MLVINGTPRKHGRTRIAASYIAALYHTDLIDLSEFVLPVFNGEADQSELLKVQELKKRVT
KADAIVLLSPEYHSGMSGALKNALDFLSSEQFKYKPVALLAVAGGGKGGINALNNMRTVM
RGVYANVIPKQLVLDPVHIDVENATVAENIKESIKELVEELSMFAKTGNPGV
SEQ ID NO: 79: NAD(P)H-Flavin Reductase, ‘Fre’, Escherichia coli
(strain K12).
MTTLSCKVTSVEAITDTVYRVRIVPDAAFSFRAGQYLMVVMDERDKRPFSMASTPDEKGF
IELHIGASEINLYAKAVMDRILKDHQIVVDIPHGEAWLRDDEERPMILIAGGTGFSYARS
ILLTALARNPNRDITIYWGGREEQHLYDLCELEALSLKHPGLQVVPVVEQPEAGWRGRTG
TVLTAVLQDHGTLAEHDIYIAGRFEMAKIARDLFCSERNAREDRLFGDAFAFI
SEQ ID NO: 80: 4-hydroxyphenylacetate 3-monooxygenase reductase
component, ‘HpaC’, Escherichia coli.
MQLDEQRLRFRDAMASLSAAVNIITTEGDAGQCGITATAVCSVTDTPPSLMVCINANSAM
NPVFQGNGKLCVNVLNHEQELMARHFAGMTGMAMEERFSLSCWQKGPLAQPVLKGSLASL
EGEIRDVQAIGTHLVYLVEIKNIILSAEGHGLIYFKRRFHPVMLEMEAAI
SEQ ID NO: 81: nitroreductase ‘NfsB’, Escherichia coli (strain
K12).
MDIISVALKRHSTKAFDASKKLTPEQAEQIKTLLQYSPSSTNSQPWHFIVASTEEGKARV
AKSAAGNYVFNERKMLDASHVVVFCAKTAMDDVWLKLVVDQEDADGRFATPEAKAANDKG
RKFFADMHRKDLHDDAEWMAKQVYLNVGNFLLGVAALGLDAVPIEGFDAAILDAEFGLKE
KGYTSLVVVPVGHHSVEDFNATLPKSRLPQNITLTEV
SEQ ID NO: 82: vanadium chloroperoxidase ‘CPO’ or ‘CiVHPO’,
Curvularia inaequalis.
MGSVTPIPLPKIDEPEEYNTNYILFWNHVGLELNRVTHTVGGPLTGPPLSARALGMLHLAIHDAYF
SICPPTDFTTFLSPDTENAAYRLPSPNGANDARQAVAGAALKMLSSLYMKPVEQPNPNPGANISDN
AYAQLGLVLDRSVLEAPGGVDRESASFMFGEDVADVFFALLNDPRGASQEGYHPTPGRYKFDDEPT
HPVVLIPVDPNNPNGPKMPFRQYHAPFYGKTTKRFATQSEHFLADPPGLRSNADETAEYDDAVRVA
IAMGGAQALNSTKRSPWQTAQGLYWAYDGSNLIGTPPRFYNQIVRRIAVTYKKEEDLANSEVNNAD
FARLFALVDVACTDAGIFSWKEKWEFEFWRPLSGVRDDGRPDHGDPFWLTLGAPATNTNDIPFKPP
FPAYPSGHATFGGAVFQMVRRYYNGRVGTWKDDEPDNIAIDMMISEELNGVNRDLRQPYDPTAPIE
DQPGIVRTRIVRHFDSAWELMFENAISRIFLGVHWRFDAAAARDILIPTTTKDVYAVDNNGATVFQ
NVEDIRYTTRGTREDPEGLFPIGGVPLGIEIADEIFNNGLKPTPPEIQPMPQETPVQKPVGQQPVK
GMWEEEQAPVVKEAP
SEQ ID NO: 83: aromatic unspecified peroxygenase ‘APO1’ or
‘AaeUPO’, Agrocybe aegerita (Black poplar mushroom) (Agaricus
aegerita).
MKYFPLFPTLVFAARVVAFPAYASLAGLSQQELDAIIPTLEAREPGLPPGPLENSSAKLVNDEAHP
WKPLRPGDIRGPCPGLNTLASHGYLPRNGVATPVQIINAVQEGLNFDNQAAVFATYAAHLVDGNLI
TDLLSIGRKTRLTGPDPPPPASVGGLNEHGTFEGDASMTRGDAFFGNNHDFNETLFEQLVDYSNRF
GGGKYNLTVAGELRFKRIQDSIATNPNFSFVDFRFFTAYGETTFPANLFVDGRRDDGQLDMDAARS
FFQFSRMPDDFFRAPSPRSGTGVEVVIQAHPMQPGRNVGKINSYTVDPTSSDFSTPCLMYEKFVNI
TVKSLYPNPTVQLRKALNTNLDFFFQGVAAGCTQVFPYGRD

1. A method of producing a reaction product, comprising:
i) contacting an oxidised flavin cofactor and molecular hydrogen (¹H₂) or an isotope thereof with a first polypeptide which is a hydrogenase enzyme or a functional fragment or derivative thereof under conditions such that the oxidised flavin cofactor is reduced to form a reduced flavin cofactor; and
ii) contacting the reduced flavin cofactor and a reactant with a second polypeptide which is an oxidoreductase or a functional fragment or derivative thereof under conditions such that (a) the oxidised flavin cofactor is regenerated; and (b) the second polypeptide catalyses the formation of the reaction product from the reactant.

2. A method according to claim 1, comprising
i) contacting an oxidised flavin cofactor and molecular hydrogen (¹H₂) or an isotope thereof with a first polypeptide which is a hydrogenase enzyme or a functional fragment or derivative thereof under conditions such that the first polypeptide oxidises the hydrogen to produce protons and electrons, and transfers the electrons to the oxidised flavin cofactor, thereby reducing the oxidised flavin cofactor to form a reduced flavin cofactor; and
ii) contacting the reduced flavin cofactor and a reactant with a second polypeptide which is an oxidoreductase or a functional fragment or derivative thereof under conditions such that (a) electrons are transferred from the reduced flavin cofactor to an electron acceptor and/or hydride ions are transferred from the reduced flavin cofactor to a hydride ion acceptor; (b) the oxidised flavin cofactor is regenerated; and (c) the second polypeptide catalyses the formation of the reaction product from the reactant.

3. A method according to claim 1 or claim 2, which comprises repeating steps (i) and (ii) of claim 1 or claim 2 multiple times thereby recycling the cofactor.

4. A method according to any one of the preceding claims, wherein the oxidised cofactor is selected from flavin mononucleotide (FMN), flavin adenine dinucleotide (FAD), riboflavin, or a derivative thereof.

5. A method according to any one of the preceding claims, wherein the first polypeptide transfers the electrons to the oxidised flavin cofactor via an intramolecular electronically-conducting pathway.

6. A method according to claim 5, wherein said intramolecular electronically-conducting pathway comprises a series of [FeS] clusters.

7. A method according to any one of the preceding claims, wherein the reduction of the oxidised flavin cofactor takes place at an [FeS] cluster within the first polypeptide.

8. A method according to any one of the preceding claims, wherein said first polypeptide does not comprise a native flavin active site for NAD(P)⁺ reduction.

9. A method according to any one of the preceding claims, wherein the first polypeptide is an uptake hydrogenase or a hydrogen-sensing hydrogenase.

10. A method according to any one of the preceding claims, wherein the first polypeptide is a hydrogenase of class 1 or 2b.

11. A method according to any one of the preceding claims, wherein the first polypeptide is selected from or comprises:
i) the amino acid sequence of Escherichia coli hydrogenase 1 (SEQ ID NOs:1 and/or 2) or an amino acid sequence having at least 60% homology therewith;
ii) the amino acid sequence of Escherichia coli hydrogenase 2 (SEQ ID NOs:3 and/or 4) or an amino acid sequence having at least 60% homology therewith;
iii) the amino acid sequence of Ralstonia eutropha membrane-bound hydrogenase moiety (SEQ ID NOs: 5 and/or 6 and/or 7) or an amino acid sequence having at least 60% homology therewith;
iv) the amino acid sequence of Ralstonia eutropha regulatory hydrogenase moiety (SEQ ID NOs: 8 and/or 9) or an amino acid sequence having at least 60% homology therewith;
v) the amino acid sequence of Aquifex aeolicus hydrogenase 1 (SEQ ID NO:10 and/or 11) or an amino acid sequence having at least 60% homology therewith;
vi) the amino acid sequence of Hydrogenovibrio marinus hydrogenase (SEQ ID NOs: 12 and/or 13) or an amino acid sequence having at least 60% homology therewith;
vii) the amino acid sequence of Thiocapsa roseopersicina hydrogenase (SEQ ID NOs: 14 and 15) or an amino acid sequence having at least 60% homology therewith;
viii) the amino acid sequence of Alteromonas macleodii hydrogenase (SEQ ID NOs: 16 and/or 17) or an amino acid sequence having at least 60% homology therewith;
ix) the amino acid sequence of Allochromatium vinosum membrane bound hydrogenase (SEQ ID NOs: 18 and/or 19) or an amino acid sequence having at least 60% homology therewith;
x) the amino acid sequence of Salmonella enterica serovar Typhimurium LT2 nickel-iron hydrogenase 5 (SEQ ID NO: 20 and/or 21) or an amino acid sequence having at least 60% homology therewith;
xi) the amino acid sequence of Desulfovibrio vulgaris Miyazaki F hydrogenase (SEQ ID NO: 23 and/or 24) or an amino acid sequence having at least 60% homology therewith;
or a functional fragment, derivative or variant thereof.

12. A method according to any one of the preceding claims, wherein the second polypeptide comprises the electron acceptor and/or hydride ion acceptor.

13. A method according to any one of the preceding claims, wherein the second polypeptide comprises a prosthetic group for oxidising the reduced flavin cofactor.

14. A method according to any one of claims 1 to 11, wherein the electron acceptor and/or hydride ion acceptor comprises a molecular substrate.

15. A method according to claim 14, wherein the molecular substrate comprises O₂.

16. A method according to any one of the preceding claims, wherein the second polypeptide is a flavin-accepting oxidoreductase, or a functional fragment, derivative or variant thereof.

17. A method according to any one of the preceding claims, wherein the second polypeptide is a flavin-dependent oxidoreductase, or a functional fragment, derivative or variant thereof.

18. A method according to claim 16 or claim 17, wherein the second polypeptide is a monooxygenase, halogenase, ene-reductase, nitro reductase, peroxidase, or haloperoxidase, or a functional fragment, derivative or variant thereof.

19. A method according to any one of claims 16 to 16, wherein the second enzyme is selected from Enzyme Commission (EC) classes 1.1.98.; 1.5.1.; 1.6.99.; 1.7.1.; 1.7.99.; 1.11.1.; 1.11.2.; 1.14.14.; 1.14.99.; 1.3.1; or a functional fragment, derivative or variant thereof.

20. A method according to any one of the preceding claims, wherein the first polypeptide and/or the second polypeptide are in solution.

21. A method according to any one of claims 1 to 19, wherein the first polypeptide and/or the second polypeptide is immobilised on a solid support.

22. A method according to any one of the preceding claims wherein the first polypeptide and the second polypeptide are attached together.

23. A method according to any one of claims 1 to 19 wherein the first polypeptide and/or the second polypeptide are comprised in a biological cell.

24. A method according to any one of the preceding claims, wherein said method is carried out under aerobic conditions.

25. A method according to any one of the preceding claims, wherein said method is carried out at a temperature of from about 20° C. to about 80° C.

26. A method of reducing an oxidised flavin cofactor, comprising:
contacting the oxidised flavin cofactor and molecular hydrogen (¹H₂) or an isotope thereof with a first polypeptide which is a hydrogenase enzyme or a functional fragment or derivative thereof under conditions such that the oxidised flavin cofactor is reduced to form a reduced flavin cofactor;
wherein the first polypeptide does not comprise a native flavin active site for NAD(P)⁺ reduction.

27. A method according to claim 26, further comprising the re-oxidation of the reduced flavin cofactor to regenerate the oxidised flavin cofactor.

28. A method according to claim 27, wherein the method steps of claim 26 and claim 27 are repeated multiple times thereby recycling the cofactor.

29. A method according to any one of claims 26 to 28, wherein:
the oxidised flavin is as defined in claim 4; and/or
the first polypeptide is as defined in any one of claims 9 to 11; and/or
said method is as defined in any one of claims 5 to 7; and/or
the first polypeptide is in solution; is immobilised on a solid support or is comprised in a biological cell; and/or
said method is carried out as defined in claim 24 or claim 25.

30. A system for reducing an oxidised flavin cofactor, comprising:
a first polypeptide which is a hydrogenase enzyme or a functional fragment or derivative thereof,
the oxidised flavin cofactor; and
molecular hydrogen (¹H₂) or an isotope thereof,
wherein the first polypeptide does not comprise a native flavin active site for NAD(P)⁺ reduction.

31. A system for producing a reaction product, comprising:
a first polypeptide which is a hydrogenase enzyme or a functional fragment or derivative thereof,
a flavin cofactor;
a second polypeptide which is an oxidoreductase or a functional fragment or derivative thereof,
molecular hydrogen (¹H₂) or an isotope thereof; and
a reactant for conversion to said reaction product.

32. A system according to claim 30 or claim 31 wherein:
the flavin cofactor is as defined in claim 4; and/or
the first polypeptide is as defined in any one of claims 9 to 11; and/or
the second polypeptide if present is as defined in any one of claims 12 to 19.


SEQUENCE LISTING (83 sequences)

SEQ ID NO: 1 - protein, 597 aa, Escherichia coli
MSTQYETQGYTINNAGRRLVVDPITRIEGHMRCEVNINDQNVITNAVSCGTMFRGLEIILQGRDPRDAWAFVERICGVCTGVHALASVYAIEDAIGIKVPDNANIIRNIMLATLWCHDHLVHFYQLAGMDWIDVLDALKADPRKTSELAQSLSSWPKSSPGYFFDVQNRLKKFVEGGQLGIFRNGYWGHPQYKLPPEANLMGFAHYLEALDFQREIVKIHAVFGGKNPHPNWIVGGMPCAINIDESGAVGAVNMERLNLVQSIITRTADFINNVMIPDALAIGQFNKPWSEIGTGLSDKCVLSYGAFPDIANDFGEKSLLMPGGAVINGDFNNVLPVDLVDPQQVQEFVDHAWYRYPNDQVGRHPFDGITDPWYNPGDVKGSDTNIQQLNEQERYSWIKAPRWRGNAMEVGPLARTLIAYHKGDAATVESVDRMMSALNLPLSGIQSTLGRILCRAHEAQWAAGKLQYFFNKLMTNLKNGNLATASTEKWEPTTWPTECRGVGFTEAPRGALGHWAAIRDGKIDLYQCVVPTTWNASPRDPKGQIGAYEAALMNTKMAIPEQPLEILRTLHSFDPCLACSTHVLGDDGSELISVQVR

SEQ ID NO: 2 - protein, 372 aa, Escherichia coli
MNNEETFYQAMRRQGVTRRSFLKYCSLAATSLGLGAGMAPKIAWALENKPRIPVVWIHGLECTCCTESFIRSAHPLAKDVILSLISLDYDDTLMAAAGTQAEEVFEDIITQYNGKYILAVEGNPPLGEQGMFCISSGRPFIEKLKRAAAGASAIIAWGTCASWGCVQAARPNPTQATSIDKVITDKPIIKVPGCPPIPDVMSAIITYMVTFDRLPDVDRMGRPLMFYGQRIHDKCYRRAHFDAGEFVQSWDDDAARKGYCLYKMGCKGPTTYNACSSTRWNDGVSFPIQSGHGCLGCAENGFWDRGSFYSRVVDIPQMGTHSTADTVGLTALGVVAAAVGVHAVASAVDQRRRHNQQPTETEHQPGNEDKQA

SEQ ID NO: 3 - protein, 552 aa, Escherichia coli
MSQRITIDPVTRIEGHLRIDCEIENGVVSKAWASGTMWRGMEEIVKNRDPRDAWMIVQRICGVCTTTHALSSVRAAESALNIDVPVNAQYIRNIILAAHTTHDHIVHFYQLSALDWVDITSALQADPTKASEMLKGVSTWHLNSPEEFTKVQNKIKDLVASGQLGIFANGYWGHPAMKLPPEVNLIAVAHYLQALECQRDANRVVALLGGKTPHIQNLAVGGVANPINLDGLGVLNLERLMYIKSFIDKLSDFVEQVYKVDTAVIAAFYPEWLTRGKGAVNYLSVPEFPTDSKNGSFLFPGGYIENADLSSYRPITSHSDEYLIKGIQESAKHSWYKDEAPQAPWEGTTIPAYDGWSDDGKYSWVKSPTFYGKTVEVGPLANMLVKLAAGRESTQNKLNEIVAIYQKLTGNTLEVAQLHSTLGRIIGRTVHCCELQDILQNQYSALITNIGKGDHTTFVKPNIPATGEFKGVGFLEAPRGMLSHWMVIKDGIISNYQAVVPSTWNSGPRNFNDDVGPYEQSLVGTPVADPNKPLEVVRTIHSFDPCMACAVH

SEQ ID NO: 4 - protein, 300 aa, Escherichia coli
MEMAESVTNPQRPPVIWIGAQECTGCTESLLRATHPTVENLVLETISLEYHEVLSAAFGHQVEENKHNALEKYKGQYVLVVDGSIPLKDNGIYCMVAGEPIVDHIRKAAEGAAAIIAIGSCSAWGGVAAAGVNPTGAVSLQEVLPGKTVINIPGCPPNPHNFLATVAHIITYGKPPKLDDKNRPTFAYGRLIHEHCERRPHFDAGRFAKEFGDEGHREGWCLYHLGCKGPETYGNCSTLQFCDVGGVWPVAIGHPCYGCNEEGIGFHKGIHQLANVENQTPRSQKPDVNAKEGGHHHHHH

SEQ ID NO: 5 - protein, 618 aa, Ralstonia eutropha
MSAYATQGFNLDDRGRRIVVDPVTRIEGHMRCEVNVDANNVIRNAVSTGTMWRGLEVILKGRDPRDAWAFVERICGVCTGCHALASVRAVENALDIRIPKNAHLIREIMAKTLQVHDHAVHFYHLHALDWVDVMSALKADPKRTSELQQLVSPAHPLSSAGYFRDIQNRLKRFVESGQLGPFMNGYWGSKAYVLPPEANLMAVTHYLEALDLQKEWVKIHTIFGGKNPHPNYLVGGVPCAINLDGIGAASAPVNMERLSFVKARIDEIIEFNKNVYVPDVLAIGTLYKQAGWLYGGGLAATNVLDYGEYPNVAYNKSTDQLPGGAILNGNWDEVFPVDPRDSQQVQEFVSHSWYKYADESVGLHPWDGVTEPNYVLGANTKGTRTRIEQIDESAKYSWIKSPRWRGHAMEVGPLSRYILAYAHARSGNKYAERPKEQLEYSAQMINSAIPKALGLPETQYTLKQLLPSTIGRTLARALESQYCGEMMHSDWHDLVANIRAGDTATANVDKWDPATWPLQAKGVGTVAAPRGALGHWIRIKDGRIENYQCVVPTTWNGSPRDYKGQIGAFEASLMNTPMVNPEQPVEILRTLHSFDPCLACSTHVMSAEGQELTTVKVR

SEQ ID NO: 6 - protein, 360 aa, Ralstonia eutropha
MVETFYEVMRRQGISRRSFLKYCSLTATSLGLGPSFLPQIAHAMETKPRTPVLWLHGLECTCCSESFIRSAHPLAKDVVLSMISLDYDDTLMAAAGHQAEAILEEIMTKYKGNYILAVEGNPPLNQDGMSCIIGGRPFIEQLKYVAKDAKAIISWGSCASWGCVQAAKPNPTQATPVHKVITDKPIIKVPGCPPIAEVMTGVITYMLTFDRIPELDRQGRPKMFYSQRIHDKCYRRPHFDAGQFVEEWDDESARKGFCLYKMGCKGPTTYNACSTTRWNEGTSFPIQSGHGCIGCSEDGFWDKGSFYDRLTGISQFGVEANADKIGGTASVVVGAAVTAHAAASAIKRASKKNETSGSEH

SEQ ID NO: 7 - protein, 244 aa, Ralstonia eutropha
MSTKMQADRIADATGTDEGAVASGKSIKATYVYEAPVRLWHWVNALAIVVLAVTGFFIGSPPATRPGEASANFLMGYIRFAHFVAAYIFAIGMLGRIYWATAGNHHSRELFSVPVFTRAYWQEVISMLRWYAFLSARPSRYVGHNPLARFAMFFIFFLSSVFMILTGFAMYGEGAQMGSWQERMFGWVIPLLGQSQDVHTWHHLGMWFIVVFVIVHVYAAIREDIMGRQSVVSTMVSGYRTFKD

SEQ ID NO: 8 - protein, 347 aa, Ralstonia eutropha
MNAPVCTGLASAKPGVLNVLWIQSGGCGGCSMSLLCADTTDFTGMLKSAGIHMLWHPSLSLESGVEQLQILEDCLQGRVALHALCVEGAMLRGPHGTGRFHLLAGTGVPMIEWVSRLAAVADYTLAVGTCAAYGGITAGGGNPTDACGLQYEGDQPGGLLGLNYRSRAGLPVINVAGCPTHPGWVTDALALLSARLLTASDLDTLGRPRFYADQLVHHGCTRNEYYEFKASAEKPSDLGCMMENMGCKGTQAHADCNTRLWNGEGSCTRGGYACISCTEPGFEEPGHPFHQTPKVAGIPIGLPTDMPKAWFVALASLSKSATPKRVKLNATADHPLIAPAIRKTRLK

SEQ ID NO: 9 - protein, 485 aa, Ralstonia eutropha
MERLVVGPFNRVEGDLEVNLEVASGRVCSARVNATMYRGLEQILLHRHPLDALVYAPRVCGICSVSQSVAASRALADLAGVTVPANGMLAMNLMLATENLADHLTHFYLFFMPDFTREIYAGRPWHTDATARFSPTHGKHHRLAIAARQRWFTLMGTLGGKWPHTESVQPGGSSRAIDAAERVRLLGRVREFRCFLEQTLYAAPLEDVVALDSEVALWRWHAQAPQAGDLRCFLTIAQDAALDQMGPGPGTYLSYGAYPQPEGGFCFAQGVWRSAQGRLDALDLAAISEDATSAWLVDQGGARHPANGLTAPAPDKVGAYTWNKAPRLAGAVLETGAIARQLAGAQPLVRDAVARCGATVYTRVLARLVELARVVPLMEDWLQSLEIGAPYWASAHLPDQGAGVGLTEAARGSLGHWVSVRDGRIDNYQIVAPTSWNFSPRDIAGQPGAVEKALEGAPVLQGETTPVAVQHIVRSFDPCMVCTVH

SEQ ID NO: 10 - protein, 633 aa, Aquifex aeolicus
MKRVVVDPVTRIEGHLRIEIMVDEETGQVKDALSAGTMWRGIELIVRNRDPRDVWAFTQRICGVCTSIHALASLRAVEDALEITIPKNANYIRNIMYGSLQVHDHVVHFYHLHALDWVSPVEALKADPVATAALANKILEKYGVLNEFMPDFLGHRAYPKKFPKATPGYFREFQKKIKKLVESGQLGIFAAHWWDHPDYQMLPPEVHLIGIAHYLNMLDVQRELFIPQVVFGGKNPHPHYIVGGVNCSISMDDMNAPVNAERLAVVEDAIYTQVESTDFFYIPDILAIADIYLNQHNWFYGGGLSKKRVIGYGDYPDEPYTGIKNGDYHKKILWHSNGVVEDFYKGVEKAKFYNLEGKDFTDPEQIQEFVTHSWYKYPDETKGLHPWDGITEPNYTGPKEGTKTHWKYLDENGKYSWIKAPRWRGKACEVGPLARYIIVYTKVKQGHIKPTWVDELIVNQIDTVSKILNLPPEKWLPTTVGRTIARALEAQMSAHTNLYWMKKLYDNIKAGDTSVANMEKWDPSTWPKEAKGVGLTEAPRGALGHWVIIKDGKVANYQCVVPTTWNGSPKDPKGQHGAFEESMIDTKVKVPEKPLEVLRGIHSFDPCLACSTHLYNEKGEEIASVRVQGVVHV

SEQ ID NO: 11 - protein, 353 aa, Aquifex aeolicus
METFWEVFKRHGVSRRDFLKFATTITGLMGLAPSMVPEVVRAMETKPRVPVLWIHGLECTCCSESFIRSATPLASDVVLSMISLEYDDTLSAAAGEAVEKHRERIIKEYWGNYILAVEGNPPLGEDGMYCIIGGRPFVEILKESAEGAKAVIAWGSCASWGCVQAAKPNPTTAVPIDKVIKDKPIIKVPGCPPIAEVMTGVIMYMVLFDRIPPLDSQGRPKMFYGNRIHDTCYRRSFFNAGQFVEQFDDEGAKKGWCLYKVGCRGPTTYNSCGNMRWYNGLSYPIQSGHGCIGCAENNFWDNGPFYERIGGIPVPGIESKADKVGAIAAAAAAGGAIIHGIASKIRKSGEKEE

SEQ ID NO: 12 - protein, 611 aa, Hydrogenovibrio marinus
MSVLNTPNHYKMDNSGRRVVIDPVTRIEGHMRCEVNVDENNVIQNAVSTGTMWRGLEVILRGRDPRDAWAFVERICGVCTGCHALASVRAVEDALDIKIPHNATLIREIMAKTLQIHDHIVHFYHLHALDWVNPVNALKADPQATSELQKLVSPHHPMSSPGYFKDIQIRIQKFVDSGQLGIFKNGYWSNPAYKLSPEADLMAVTHYLEALDFQKEIVKIHAIFGGKNPHPNYMVGGVPCAINIDGDMAAGAPINMERLNFVKSLIEQGRTFNTNVYVPDVIAIAAFYRDWLYGGGLSATNVMDYGAYPKTPYDKSTDQLPGGAIINGDWGKIHPVDPRDPEQVQEFVTHSWYKYPDETKGLHPWDGITEPNYELGSKTKGSRTNIIEIDESAKYSWIKSPRWRGHAVEVGPLARYILAYAQGVEYVKTQVHTSLNRFNAVCRLLDPNHKDITDLKAFLGSTIGRTLARALESEYCGDMMLDDFNQLISNIKNGDSSTANTDKWDPSSWPEHAKGVGTVAAPRGALAHWIVIEKGKIKNYQCVVPTTWNGSPRDPKGNIGAFEASLMGTPMERPDEPVEVLRTLHSFDPCLACSTHVMSEEGEEMATVKVR

SEQ ID NO: 13 - protein, 361 aa, Hydrogenovibrio marinus
MSSQVETFYEVMRRQGITRRSFLKYCSLTAAALGLSPAYANKIAHAMETKPRTPVIWLHGLECTCCSESFIRSAHPLAKDVVLSMISLDYDDTLMAASGHAAEAILDEIKEKYKGNYILAVEGNPPLNQDGMSCIIGGRPFSEQLKRMADDAKAIISWGSCASWGCVQAAKPNPTQATPVHKFLGGGYDKPIIKVPGCPPIAEVMTGVITYMLTFDRIPELDRQGRPKMFYSQRIHDKCYRRPHFDAGQFVEEWDDEGARKGYCLYKVGCKGPTTYNACSTVRWNGGTSFPIQSGHGCIGCSEDGFWDKGSFYSRDTEMNAFGIEATADDIGKTAIGVVGAAVVAHAAISAVKAAQKKGDK

SEQ ID NO: 14 - protein, 596 aa, Thiocapsa roseopersicina
MSVTTANGFELDTAGRRLVVDPVTRIEGHLRCEVNLDENNVIRNAVSTGTMWRGLEVILRGRDPRDAWAFTERICGVCTGTHALTSVRAVEDALGIPIPENANSIRNIMHVTLQAHDHLVHFYHLHALDWVDVVSALGADPKATSALAQSISDWPKSSPGYFRDVQNRLKRFVESGQLGPFMNGYWGSPAYKLPPEANLMAVTHYLEALDFQKEIVKIHTVYGGKNPHPNWLVGGMPCAINVDGTGAVGAINMERLNLVSSIIDQTIAFIDKVYIPDLIAIASFYKDWTYGGGLSSQAVMSYGDIPDHANDMSSKNLLLPRGAIINGNLNEIHEIDLRNPEEIQEFVDHSWFSYKDETRGLHPWDGVTEPNFVLGPNAVGSRTRIEALDEQAKYSWIKAPRWRGHAMEVGPLARYVIGYAKGIPEFKEPVDKVLTDLGQPLEAIFSTLGRTAARGLEASWAAHKMRYFQDKLVANIRAGDTATANVDNWDPKTWPKEARGVGTTEAPRGALGHWIVIKDGKIDNYQAVVPTTWNGSPRDPAGNIGAFEASLLNTPLAKADEPLEILRTLHSFDPCLACATHIMGPDGEELTRIKVR

SEQ ID NO: 15 - protein, 360 aa, Thiocapsa roseopersicina
MPTTETYYEVMRRQGITRRSFLKFCSLTATALGLSPTFAGKIAHAMETKPRIPVVWLHGLECTCCSESFIRSAHPLVSDVILSMISLDYTILIMAAAGHQAEAILEEVRHKHAGNYILAVEGNPPLNQDGMSCIIGGRPFLEQLLEMADSCKAVISWGSCASWGCVQAARPNPTRATPVHEVIRDKPVIKVPGCPPIAEVMTGVLTYILTFDRLPELDRQGRPLMFYGQRIHDKCYRRPHFDAGQFVESWDDEGARRGYCLYKVGCKGPTTYNACSTIRWNGGVSFPIQSGHGCIGCSEDGFWDKGSFYQHVTDTHAFGIEANADRTGIAVATRRGAAHRAHAAVSVVKRVQQKKEEDQS

SEQ ID NO: 16 - protein, 332 aa, Alteromonas macleodii
MALPTLNKQLQASGISRRTFLKFCATTASLLALPQSAVADLATALGNARRPSVIWLPFQECTGCTEAILRSHAPTLESLIFDHISLDYQHTIMAAAGEQAEDARRAAMNAHKGQYLLLVDGSVPVGNPGYSTISGMSNVDMLRESAKDAAGIIAIGTCASFGGIPKANPNPTGAVAVSDIITDKPIVNISGCPPLPIAITAVLVHYLTFKRFPDLDELQRPLAFFGESIHDRCYRRPFFEQRKFAKSFDDEGAKNGWCLFELGCKGPETFNACATVKWNQGTSFPIESGHPCLGCSEPDFWDKSSFYQALGPWEWYKSKPGKGAQKHAGKNS

SEQ ID NO: 17 - protein, 625 aa, Alteromonas macleodii
MENTASNNRLVVDPITRIEGHLRIEAEMDGNTIKQAFSSGTSVRGIELILQGRDPRDAWAFAQRICGVCTLVHGMASVRAVEDAIRKAWRSNAKLGVAIGKPSMTSMPKGPMQHGKKGHRQSRTSIGVLSEAEMAIPQNAQLIRNIMIATQYVHDHVMHFYHLHALDWVDVVSALDADPTRTATLAGQLSDYPRSSPGYFKDVKQKVKTLVESGQLGIFSNAYWGHPGYKLPPEVNLMALAHYLDALTWQREVVKVHTIFGGKNPHPNFVVGGVPSPINLNASTGINTSRLVQLQDAITQMKSFVDQVYYPDIVAIAGYYKEWGTRGEGLGNFLTYGDLPMTSMDDPDSFLFPRGAILGRDLSKVHDLDLDDPSEIQEFVSSSWYRYSGGNASGLHPFNGQTTLEYTGPKPPYKHLNVGAEYSWLKSPRWKGHAMEVGPLARVLMMYAKKDAAAQDIVNRSLSILDLETSALFSTLGRTLARAVETKIVVNQLQSWYDQLLDNIAKGDTDTFNPLYFDPTNWPIKGQGVGVMEAPRGALGHWLVMQNGKIENYQCVVPTTWNAGPRDPNSQAGAYEAALQDKHTLHDPDQPLEILRTLHSFDPCLACAVHVMDETGEERLRLKVR

SEQ ID NO: 18 - protein, 576 aa, Allochromatium vinosum
MSERIVVDPVTRIEGHLRIEAQMDGENIAQAYSSGTSVRGLETILKGRDPRDAWAFAQRICGVCTLVHGIASVRSVEDALKIELPPNAQLIRNLMISSQFVHDHVMHFYHLHALDWVDVVSALSADPKATSDLAQSISSWPKSSPGYFADTQKRIKTFVESGQLGIFANGYWGHPAYKLPPEANLMAVAHYLEALAWQRDVARLHAIFGGKNPHPNFVVGGVPSPIDIDSDSAINAKRLAEVQQILQSMQTFVDQVYVPDTLAIASFYKDWGERGEGLGNFMSYGDLPATGTMDPAQFLFPRGVILNRDLSTIHEIDLHDAGQIQEYVAHSWYEYSGGNDQGLHPYDGETNLEYDARGGVKPPYTQLDVNDGYSWMKAPRWKGHAMEVGPLARVLLLYASGHEQTKELVEMTLTTLDLPVRALYSTLGRTAARTLETKILTDTAQDWYNQLIANIKAGDSRTFNETLWEPSSWPAEARGAGYMEAPRGALGHWIVIKDRKIANYQAVVPSTWNAGPRDPSDQPGAYEAALQDNHQLVDVKQPIEILRTIHSFDPCIACAVHLTDPETGEQMEIKIT

SEQ ID NO: 19 - protein, 314 aa, Allochromatium vinosum
PSVVWLSFQECTGCTESLTRAHAPTLEDLILDFISLDYHHTLQAASGEAAEAARLQAMDENRGQYLVIVDGSIPGPDANPGFSTVAGHSNYSILMETVEHAAAVIAVGTCAAFGGLPQARPNPTGAMSVMDLVRDKPVINVPGCPPIPMVITGVIAHYLVFGRLPEVDGYGRPLAFYGQSIHDRCYRRPFYDKGLFAESFDDEGAKQGWCLYRLGCKGPTTYNACATMKWNDGTSWPVEAGHPCLGCSEPQFWDAGGFYEPVSVPLTLGPATLLGAGAAGAVVGGGLAALSRKKGRDAAATRQPVTVDELEQKL

SEQ ID NO: 20 - protein, 585 aa, Salmonella enterica
MAYPYQTQGFTLDNSGRRIVVDPVTRIEGHMRCEVNIDSNNVITNAVSTGTMWRGLEVILKGRDPRDAWAFVERICGVCTGTHALTSIRAVENALGIAIPDNANCIRNMMQATLHVHDHLVHFYHLHALDWVDVVAALKADPHQTSAIAQSLSAWPLSSPGYFRDLQNRLKRFIESGQLGPFRNGYWGHPAMKLPPEANLLAVAHYLEALDFQKEIVKIHTVFGGKNPHPNWLVGGVPCAINLDETGAVGAVNMERLNLVSSIIQKARQFCEQVYLPDVLLIASYYKDWAKIGGGLSSMNLLAYGEFPDNPNDYSASNLLLPRGAIINGRFDEIHPVDLTAPDEIQEFVTHSWYTYGNGNNDKGLHPWDGLTEPQLVMGEHYKGTKTFIEQVDESAKYSWIKSPRWKGHAMEVGPLARYLIGYHQNKPEFKEPVDQLLSVLKLPKEALFSTLGRTAARALESVWAGNTLQYFFDRLMRNLKSGDTATANVTLWEPDTWPTSAKGVGFSEAPRGALGHWIKIANQKIDSYQCVVPTTWNAGPRDDKGQIGAYEAALMGTKLAVPDQPLEILRTLHSFDPCLACSTH

SEQ ID NO: 21 - protein, 279 aa, Salmonella enterica
LENKPRTPVIWLHGLECTCCTESFIRSAHPLAKDAILSLISLDYDDTIMAAAGQQAEQALADVMREYKGNYIVAVEGNAPLNEDGMFCILAGEPFLEKLKRVSADAKAIIAWGSCASWGCVQAARPNPTKATPVHKLITDKPIIKVPGCPPIPEVMSAVITYMLAFDRIPPLDRLGRPKMFYGQRIHDKCYRRAHFDAGQFVEAWDDEGARKGYCLYKMGCKGPTTYNACSTVRWNDGVSFPIQSGHGCLGCSEDGFWDYGSFYSRATGSRSHHHHHHH

SEQ ID NO: 22 - protein, 235 aa, Escherichia coli
MQQKSDNVVSHYVFEAPVRIWHWLTVLCMAVLMVTGYFIGKPLPSVSGEATYLFYMGYIRLIHFSAGMVFTVVLLMRIYWAFVGNRYSRELFIVPVWRKSWWQGVWYEIRWYLFLAKRPSADIGHNPIAQAAMFGYFLMSVFMIITGFALYSEHSQYAIFAPFRYVVEFFYWTGGNSMDIHSWHRLGMWLIGAFVIGHVYMALREDIMSDDTVISTMVNGYRSHKFGKISNKERS

SEQ ID NO: 23 - protein, 534 aa, Desulfovibrio vulgaris
SSYSGPIVVDPVTRIEGHLRIEVEVENGKVKNAYSSSTLFRGLEIILKGRDPRDAQHFTQRTCGVCTYTHALASTRCVDNAVGVHIPKNATYIRNLVLGAQYLHDHIVHFYHLHALDFVDVTAALKADPAKAAKVASSISPRKTTAADLKAVQDKLKTFVETGQLGPFTNAYFLGGHPAYYLDPETNLIATAHYLEALRLQVKAARAMAVFGAKNPHTQFTVVGGVTCYDALTPQRIAEFEALWKETKAFVDEVYIPDLLVVAAAYKDWTQYGGTDNFITFGEFPKDEYDLNSRFFKPGVVFKRDFKNIKPFDKMQIEEHVRHSWYEGAEARHPWKGQTQPKYTDLHGDDRYSWMKAPRYMGEPMETGPLAQVLIAYSQGHPKVKAVTDAVLAKLGVGPEALFSTLGRTAARGIETAVIAEYVGVMLQEYKDNIAKGDNVICAPWEMPKQAEGVGFVNAPRGGLSHWIRIEDGKIGNFQLVVPSTWTLGPRCDKNKLSPVEASLIGTPVADAKRPVEILRTVHSFDPCIACGVH

SEQ ID NO: 24 - protein, 267 aa, Desulfovibrio vulgaris
LMGPRRPSVVYLHNAECTGCSESVLRAFEPYIDTLILDTLSLDYHETIMAAAGDAAEAALEQAVNSPHGFIAVVEGGIPTAANGIYGKVANHTMLDICSRILPKAQAVIAYGTCATFGGVQAAKPNPTGAKGVNDALKHLGVKAINIAGCPPNPYNLVGTIVYYLKNKAAPELDSLNRPTMFFGQTVHEQCPRLPHFDAGEFAPSFESEEARKGWCLYELGCKGPVTMNNCPKIKFNQTNWPVDAGHPCIGCSEPDFWDAMTPFYQN

SEQ ID NO: 25-30 - skipped (6 entries)

SEQ ID NO: 31 - protein, 349 aa, Thermus scotoductus [sequence omitted]

SEQ ID NO: 32 - protein, 400 aa, Saccharomyces pastorianus
MSFVKDFKPQALGDTNLFKPIKIGNNELLHRAVIPPLTRMRALHPGNIPNRDWAVEYYTQRAQRPGTMIITEGAFISPQAGGYDNAPGVWSEEQMVEWTKIFNAIHEKKSFVWVQLWVLGWAAFPDNLARDGLRYDSASDNVFMDAEQEAKAKKANNPQHSLTKDEIKQYIKEYVQAAKNSIAAGADGVEIHSANGYLLNQFLDPHSNTRTDEYGGSIENRARFTLEVVDALVEAIGHEKVGLRLSPYGVFNSMSGGAETGIVAQYAYVAGELEKRAKAGKRLAFVHLVEPRVTNPFLTEGEGEYEGGSNDFVYSIWKGPVIRAGNFALHPEVVREEVKDKRTLIGYGRFFISNPDLVDRLEKGLPLNKYDRDTFYQMSAHGYIDYPTYEEALKLGWDKK

SEQ ID NO: 33 - protein, 400 aa, Saccharomyces cerevisiae strain ATCC 204508 / S288c
MPFVKDFKPQALGDTNLFKPIKIGNNELLHRAVIPPLTRMRAQHPGNIPNRDWAVEYYAQRAQRPGTLIITEGTFPSPQSGGYDNAPGIWSEEQIKEWTKIFKAIHENKSFAWVQLWVLGWAAFPDTLARDGLRYDSASDNVYMNAEQEEKAKKANNPQHSITKDEIKQYVKEYVQAAKNSIAAGADGVEIHSANGYLLNQFLDPHSNNRTDEYGGSIENRARFTLEVVDAVVDAIGPEKVGLRLSPYGVFNSMSGGAETGIVAQYAYVLGELERRAKAGKRLAFVHLVEPRVTNPFLTEGEGEYNGGSNKFAYSIWKGPIIRAGNFALHPEVVREEVKDPRTLIGYGRFFISNPDLVDRLEKGLPLNKYDRDTFYKMSAEGYIDYPTYEEALKLGWDKN

SEQ ID NO: 34 - protein, 338 aa, Bacillus subtilis
MARKLFTPITIKDMTLKNRIVMSPMCMYSSHEKDGKLTPFHMAHYISRAIGQVGLIIVEASAVNPQGRITDQDLGIWSDEHIEGFAKLTEQVKEQGSKIGIQLAHAGRKAELEGDIFAPSAIAFDEQSATPVEMSAEKVKETVQEFKQAAARAKEAGFDVIEIHAAHGYLIHEFLSPLSNHRTDEYGGSPENRYRFLREIIDEVKQVWDGPLFVRVSASDYTDKGLDIADHIGFAKWMKEQGVDLIDCSSGALVHADINVFPGYQVSFAEKIREQADMATGAVGMITDGSMAEEILQNGRADLIFIGRELLRDPFFARTAAKQLNTEIPAPVQYERGW

SEQ ID NO: 35 - protein, 363 aa, Pseudomonas putida
MSALFEPYTLKDVTLRNRIAIPPMCQYMAEDGMINDWHHVHLAGLARGGAGLLVVEATAVAPEGRITPGCAGIWSDAHAQAFVPVVQAIKAAGSVPGIQIAHAGRKASANRPWEGDDHIAADDTRGWETIAPSAIAFGAHLPKVPREMTLDDIARVKQDFVDAARRARDAGFEWIELHFAHGYLGQSFFSEHSNKRTDAYGGSFDNRSRFLLETLAAVREVWPENLPLTARFGVLEYDGRDEQTLEESIELARRFKAGGLDLLSVSVGFTIPDTNIPWGPAFMGPIAERVRREAKLPVTSAWGFGTPQLAEAALQANQLDLVSVGRAHLADPHWAYFAAKELGVEKASWTLPAPYAHWLERYR

SEQ ID NO: 36 - protein, 354 aa, ‘Ferrovum’ strain JA12
MSLLFSPYQLGSLSLANRLVIAPMCQYSAVDGIAQDWHLMHLGRLAISGAGLVIVEATGVNPEGRITPFCLGLYNDEQEAALGRIVAFAREFGQAKMAIQLAHAGRKASTRRPWDPGSPYSPEEGGWQTWAPSAIKFYEESLTPHPMSIEDLETVKQDFVNSAIRAERAGFKAIELHGAHGYLIHQFLSPLSNQRQDQYGGSLENRMRYPLEILSAVKHALSAEMVVGMRISAVDWAPGGLTIEESITFSQECEKRGAGFIHVSTGGLVAHQQIPVGPGYQVEHAQAIKQNVNIPTMAVGLITHSAQAETILKSEQADMIAIARAALKNPHWPWTAALELGDKPFAPPQYQRAR

SEQ ID NO: 37 - protein, 405 aa, Meyerozyma guilliermondii
MSVNINPLGETQVFQPIKLGKNTLSHRVFFPPTTRTRSLEDHTPSNLAYKYYDERSKFPGTLIISEGTFPSAQAGLYEGVPGIWTERQTKTWKHIIDKIHENKSFASIQLWNLGRTGDPALLKKAGKPFLAPSAIYFDEESKKAAEKAGNPLRAMTEEEIKDMIYEQYTIAAKNALEAGFDYIELHSAHGYLLHEFLEESSNKRTDKYGGSIENRARFVLELVDHMISIVGAERLGIRISPWATFQGMKSVHGEVHPLTTYSYLVNELEKRAQAGNRLAYISLVEPRVDGINSVEKKDQTGNNDFVKDLWKGTILKAGNYTYDAPKFGQLLDDVSDGRTLVGFSRYFISNPDLISRLEKGHQLAPYERETFYGRSDFGYNDYPKYGEKREDAEVAKKRVPEELVV

SEQ ID NO: 38 - protein, 406 aa, Clavispora lusitaniae
MVAVKPLKDTEIFKPTKVGNHELSNKIVYAPTTRMRAIADHTPSDLAYKYYDDRTKYPGSLVITEATLMSPKTGLYDRVPGIYTDEHVAGWKKITDKIHANGSKVSMQLWPLGRVADPVATKKAGYPLVAPSLIYPSEEAKKAAEEAGNPIHVLTTEEVEDLVNDFVHAAKKAVAAGVDYVEVHGAHGYLVDTFFQVSTNKRTDKYGGSIENRARFALEILDRLIEEIGAERVAIRISPWAKFQGILAEEGEVNPVAQFGYFLSELENRARAGKRIAYVSIVEPRVSGVIDVAGEDIQGDNSFVRSVWKGIVIKAGNYTYDAPEFKTLLQDVSDGKTLVAFARYFTSNPDLVQRLHDGADLTPYKRELFYAPSNWGYNTFTNAGETKTFSEEEESKRLPAPIDTKA

SEQ ID NO: 39 - protein, 522 aa, Chondromyces crocatus
MQLPSSTKILVVGGGPAGSTAATLLAREGFEVTLVEKAIFPRYHIGESLLISVQPIIDLLGAREAVEAHGFQRKKGVLWEWGGERWLFDWKKLRYDYTFHVKREEFDEILLRNAQKNGVKVFEGIDISRLEFDGERPVAAKWSKSSTGESGTIQFEFLLDASGRAGLMATQYLRSRMFMKAFQNVATWGYWKGATIPEVEVEGPITVGSIPYGWIWGIPLRDQTMSVGLVIHQELFKEKRATQSVEEIYHEGLKASPLFQDVVLKGATLEPQIRTETDYSYISRTLAGPGFFLVGDSGAFIDPLLSSGVHLAMHSALLAAASVKSIIAGEVDMASATEFYQRCYQGHFLRWALIVASFYEVNARKETYFWTAQQLAHEELGVFNMSQADMKDVFATMVSGVVDLGDAQNAGRLQKGAERVHQYLDDDGREEDVTALLQKSKQRIFEYLDRVKNRDSRAAMQRYKAGGTETFSMGLDADGAVGGLYVTTEPRLGLLRKVVEERAEAATEAPAPAAPPPAVAEV

SEQ ID NO: 40 - protein, 511 aa, Streptomyces rugosporus
MIRSVVIVGGGTAGWMTASYLKAAFDDRIDVTLVESGNVRRIGVGEATFSTVRHFFDYLGLDEREWLPRCAGGYKLGIRFENWSEPGEYFYHPFERLRVVDGFNMAEWWLAVGDRRTSFSEACYLTHRLCEAKRAPRMLDGSLFASQVDESLGRSTLAEQRAQFPYAYHFDADEVARYLSEYAIARGVRHVVDDVQHVGQDERGWISGVHTKQHGEISGDLFVDCTGFRGLLINQTLGGRFQSFSDVLPNNRAVALRVPRENDEDMRPYTTATAMSAGWMWTIPLFKRDGNGYVYSDEFISPEEAERELRSTVAPGRDDLEANHIQMRIGRNERTWINNCVAVGLSAAFVEPLESTGIFFIQHAIEQLVKHFPGERWDPVLISAYNERMAHMVDGVKEFLVLHYKGAQREDTPYWKAAKTRAMPDGLARKLELSASHLLDEQTIYPYYHGFETYSWITMNLGLGIVPERPRPALLHMDPAPALAEFERLRREGDELIAALPSCYEYLASIQ

SEQ ID NO: 41 - protein, 530 aa, Lechevalieria aerocolonigenes
MSGKIDKILIVGGGTAGWMAASYLGKALQGTADITLLQAPDIPTLGVGEATIPNLQTAFFDFLGIPEDEWMRECNASYKVAIKFINWRTAGEGTSEARELDGGPDHFYHSFGLLKYHEQIPLSHYWFDRSYRGKTVEPFDYACYKEPVILDANRSPRRLDGSKVTNYAWHFDAHLVADFLRRFATEKLGVRHVEDRVEHVQRDANGNIESVRTATGRVFDADLFVDCSGFRGLLINKAMEEPFLDMSDHLLNDSAVATQVPHDDDANGVEPFTSAIAMKSGWTWKIPMLGRFGTGYVYSSRFATEDEAVREFCEMWHLDPETQPLNRIRFRVGRNRRAWVGNCVSIGTSSCFVEPLESTGIYFVYAALYQLVKHFPDKSLNPVLTARFNREIETMFDDTRDFIQAHFYFSPRTDTPFWRANKELRLADGMQEKIDMYRAGMAINAPASDDAQLYYGNFEEEFRNFWNNSNYYCVLAGLGLVPDAPSPRLAHMPQATESVDEVFGAVKDRQRNLLETLPSLHEFLRQQHGR

SEQ ID NO: 42 - protein, 538 aa, Pseudomonas fluorescens
MNKPIKNIVIVGGGTAGWMAASYLVRALQQQANITLIESAAIPRIGVGEATIPSLQKVFFDFLGIPEREWMPQVNGAFKAAIKFVNWRKSPDPSRDDHFYHLFGNVPNCDGVPLTHYWLRKREQGFQQPMEYACYPQPGALDGKLAPCLSDGTRQMSHAWHFDAHLVADFLKRWAVERGVNRVVDEVVDVRLNNRGYISNLLTKEGRTLEADLFIDCSGMRGLLINQALKEPFIDMSDYLLCDSAVASAVPNDDARDGVEPYTSSIAMNSGWTWKIPMLGRFGSGYVFSSHFTSRDQATADFLKLWGLSDNQPLNQIKFRVGRNKRAWVNNCVSIGLSSCFLEPLESTGIYFIYAALYQLVKHFPDTSFDPRLSDAFNAEIVHMFDDCRDFVQAHYFTTSRDDTPFWLANRHDLRLSDAIKEKVQRYKAGLPLTTTSFDDSTYYETFDYEFKNFWLNGNYYCIFAGLGMLPDRSLPLLQHRPESIEKAEAMFASIRREAERLRTSLPTNYDYLRSLRDGDAGLSRGQRGPKLAAQESL

SEQ ID NO: 43 - protein, 513 aa, Streptomyces violaceusnige
LNNVVIVGGGTAGWMTASYLKAAFGDRIDITLVESGHIGAVGVGEATFSDIRHFFEFLGLKEKDWMPACNATYKLAVRFENWREKGHYFYHPFEQMRSVNGFPLTDWWLKQGPTDRFDKDCFVMASVIDAGLSPRHQDGTLIDQPFDEGADEMQGLTMSEHQGKTQFPYAYQFEAALLAKYLTKYSVERGVKHIVDDVREVSLDDRGWITGVRTGEHGDLTGDLFIDCTGFRGLLLNQALEEPFISYQDTLPNDSAVALQVPMDMERRGILPCTTATAQDAGWIWTIPLTGRVGTGYVYAKDYLSPEEAERTLREFVGPAAADVEANHIRMRIGRSRNSWVKNCVAIGLSSGFVEPLESTGIFFIHHAIEQLVKNFPAADWNSMHRDLYNSAVSHVMDGVREFLVLHYVAAKRNDTQYWRDTKTRKIPDSLAERIEKWKVQLPDSETVYPYYHGLPPYSYMCILLGMGGIELKPSPALALADGGAAQREFEQIRNKTQRLTEVLPKAYDYFTQ

SEQ ID NO: 44 - protein, 523 aa, Streptomyces toxytricini
MNTRNPDKVVIVGGGTAGWMTASYLKKAFGERVSVTLVESGTIGTVGVGEATFSDIRHFFEFLDLREEEWMPACNATYKLAVRFQDWQRPGHHFYHPFEQMRSVDGFPLTDWWLQNGPTDRFDRDCFVMASLCDAGRSPRYLNGSLLQQEFDERAEEPAGLTMSEHQGKTQFPYAYHFEAALLAEFLSGYSKDRGVKHVVDEVLEVKLDDRGWISHVVTKEHGDIGGDLFVDCTGFRGVLLNQALGVPFVSYQDTLPNDSAVALQVPLDMEARGIPPYTRATAKEAGWIWTIPLIGRIGTGYVYAKDYCSPEEAERTLREFVGPEAADVEANHIRMRIGRSEQSWKNNCVAIGLSSGFVEPLESTGIFFIHHAIEQLVKHFPAGDWHPQLRAGYNSAVANVMDGVREFLVLHYLGAARNDTRYWKDTKTRAVPDALAERIERWKVQLPDSENVFPYYHGLPPYSYMAILLGTGAIGLRPSPALALADPAAAEKEFTAIRDRARFLVDTLPSQYEYFAAMGQRV

SEQ ID NO: 45 - protein, 533 aa, Kutzneria sp. 744
MDDNRIRSILVLGGGTAGWMSACYLSKALGPGVEVTVLEAPSISRIRVGEATIPNLHKVFFDFLGIAEDEWMRECNASYKAAVRFVNWRTPGDGQATPRRRPDGRPDHFDHLFGQLPEHENLPLSQYWAHRRLNGLTDEPFDRSCYVQPELLDRKLSPRLMDGTKLASYAWHFDADLVADFLCRFAVQKLNVTHVQDVFTHADLDQRGHITAVNTESGRTLAADLFIDCSGFRSVLMGKVMQEPFLDMSKHLLNDRAVALMLPHDDEKVGIEPYTSSLAMRSGWSWKIPLLGRFGSGYVYSSQFTSQDEAAEELCRMWDVDPAEQTFNNVRFRVGRSRRAWVRNCVAIGVSAMFVEPLESTGLYFSYASLYQLVKHFPDKRFRPILADRFNREVATMYDDTRDFLQAHFSLSPRDDSEFWRACKELPFADGFAEKVEMYRAGLPVELPVTIDDGHYYGNFEAEFRNFWTNSNYYCIFAGLGFLPEHPLPVLEFRPEAVDRAEPVFAAVRRRTEELVATAPTMQAYLRRLHQGT

SEQ ID NO: 46 - protein, 567 aa, Pseudomonas fluorescens
MTQKSPANEHDSNHFDVIILGSGMSGTQMGAILAKQQFRVLIIEESSHPRFTIGESSIPETSLMNRIIADRYGIPELDHITSFYSTQRYVASSTGIKRNFGFVFHKPGQEHDPKEFTQCVIPELPWGPESHYYRQDVDAYLLQAAIKYGCKVHQKTTVTEYHADKDGVAVTTAQGERFTGRYMIDCGGPRAPLATKFKLREEPCRFKTHSRSLYTHMLGVKPFDDIFKVKGQRWRWHEGTLHHMFEGGWLWVIPFNNHPRSTNNLVSVGLQLDPRVYPKTDISAQQEFDEFLARFPSIGAQFRDAVPVRDWVKTDRLQFSSNACVGDRYCLMLHANGFIDPLFSRGLENTAVTIHALAARLIKALRDDDFSPERFEYIERLQQKLLDHNDDFVSCCYTAFSDFRLWDAFHRLWAVGTILGQFRLVQAHARFRASRNEGDLDHLDNDPPYLGYLCADMEEYYQLFNDAKAEVEAVSAGRKPADEAAARIHALIDERDFAKPMFGFGYCITGDKPQLNNSKYSLLPAMRLMYWTQTRAPAEVKKYFDYNPMFALLKAYITTRIGLALKK

SEQ ID NO: 47 - protein, 455 aa, Pseudomonas protegens Pf-5
GPHMSDHDYDVVIIGGGPAGSTMASYLAKAGVKCAVFEKELFEREHVGESLVPATTPVLLEIGVMEKIEKANFPKKFGAAWTSADSGPEDKMGFQGLDHDFRSAEILFNERKQEGVDRDFTFHVDRGKFDRILLEHAGSLGAKVFQGVEIADVEFLSPGNVIVNAKLGKRSVEIKAKMVVDASGRNVLLGRRLGLREKDPVFNQFAIHSWFDNFDRKSATQSPDKVDYIFIHFLPMTNTWVWQIPITETITSVGVVTQKQNYTNSDLTYEEFFWEAVKTRENLHDALKASEQVRPFKKEADYSYGMKEVCGDSFVLIGDAARFVDPIFSSGVSVALNSARIASGDIIEAVKNNDFSKSSFTHYEGMIRNGIKNWYEFITLYYRLNILFTAFVQDPRYRLDILQLLQGDVYSGKRLEVLDKMREIIAAVESDPEHLWHKYLGDMQVPTAKPAFEND

SEQ ID NO: 48 - protein, 502 aa, Pseudomonas fluorescens (strain ATCC BAA-477 / NRRL B-23932 / Pf-5)
MNQYDVIIIGSGIAGALTGAVLAKSGLNVLILDSAQHPRFSVGEAATPESGFLLRLLSKRFDIPEIAYLSHPDKIIQHVGSSACGIKLGFSFAWHQENAPSSPDHLVAPPLKVPEAHLFRQDIDYFALMIALKHGAESRQNIKIESISLNDDGVEVALSNAAPVKAAFIIDAAAQGSPLSRQLGLRTTEGLATDTCSFFTHMLNVKSYEDALAPLSRTRSPIELFKSTLHHIFEEGWLWVIPFNNHPQGTNQLCSIGFQFNNAKYRPTEAPEIEFRKLLKKYPAIGEHFKDAVNAREWIYAPRINYRSVQNVGDRFCLLPQATGFIDPLFSRGLITTFESILRLAPKVLDAARSNRWQREQFIEVERHCLNAVATNDQLVSCSYEAFSDFHLWNVWHRVWLSGSNLGSAFLQKLLHDLEHSGDARQFDAALEAVRFPGCLSLDSPAYESLFRQSCQVMQQAREQARPVAETANALHELIKEHEAELLPLGYSRISNRFILKV

SEQ ID NO: 49 - protein, 448 aa, Streptomyces sp. CNH-287
MSEVDFDIGIIGGGPAGSAIASYLAKAGLNCVIFEGDIFPREHVGESLIPATTPVLDDIGFLPQMETAGFPKKYGAAWTSASDQNIPTMGFEGMSHGFRAAEIEFHEREQLGVTQDYTYHVDRAKFDLMLLQHAASLGAKVYQGVRVRRVDFSETNPRIIFPIGKTETSVRVRMVVDASGRNTFLGRQLKLKVSDPVFNQYAVHTWFDNLDRTALSVNKEQADYIFIHFLPVTDTWVWQIPITDTITSIGVVTQKEQFKASKEDLDGFFWECVGSRPELKEALEKSDQVRPFKTEGDYSYAMKQIVGDQWATIGDAARFVDPIFSSGVSVALNSARLISGEIIAAAEQGDFRKEMFSNYEGKIRRAVSNWYEFISIYYRLNILFTAFVQDPRYRLDVLKMLQGDVYDDEEPKALAAMREITKQVEENPDHLWHKHLGSLRAPSAAPMF

SEQ ID NO: 50 - protein, 406 aa, Pseudoalteromonas piscicida
MNGFTHYDVVIIGSGPAGSLCGIECRKKGMSVLCIEKEQFPRFHIGESLTGNAGQIIRDLGLADAMDAAGFPDKPGVNVIGSLSKNEFFIPILAPTWQVRRSDFDNMIKQKAVEHGVEYQLGMVTDVIREGDKVVGAVYKADGIEHQVRSKVLVDASGQNTFLSRKGIAGKRQIEFFSQQIASFAHYKGVERDLPPFSTNTTILYSKQYHWSWIIPISPDTDSLGVVIPKDLYYKECKNPDDAIEWGMEHISPELKRRFKNAERQGESQSMADFSYRIEPFVGDGWLCIGDAHRFLDPIFSYGVSFAMKEGIRAAEAIEQVVNGQDWKAPFYAYRDWSNGGQQIAADLIRYFWIYPIFFGYQMQNPDLRDEVIRLLGGCCFDCKDWKAPAIFRNAIEEYDRKQMAS

SEQ ID NO: 51 - protein, 519 aa, Metacordyceps chlamydosporia
MSVPKSCTILVAGGGPAGSYAAAALAREGNDVVLLEADQHPRYHIGESMLPSLRPLLRFIDLEDKFDAHGFQKKLGAAFKLTSKREGSHGPRGYSWNVVRSESDEILFNHARESGAQAFQGIKINAIEFEPYEEEYPNGEKVANPGKPTSAKWSSKDGSSGDIAFKYLVDATGRIGIMSTKYLKNRHYNEGLKNLAIWGYYKNNIPWAQGTPRENQPFFEGMRDGAGWCWTIPLHNGTVSVGAVMRKDLFFEKKKSLGENATNTQIMAECMKLCPTIGELLAPAELVSDIKQATDYSYSATAYAGPNFRIVGDAGCFIDPFFSSGHHLAVAGALAAAVSINASIKGDCTEYEASRWHAKKVDEGYTLFLLVVMAALKQIRMQENPVLSDVDEDGFDRAFQFLRPEAVKKFTKEDVAQTIDFAVHALNNMAELDMDIPEHMINGDKEGENGVTNGNNGAAKTAGLASNMEKLTNDEEKVLNGLRILGKAAPGGTLADFEGTAIDGLMPRLEHGKLGLNKV

SEQ ID NO: 52 - protein, 529 aa, Artificial Sequence
  the amino acid sequence of Tryptophan 6-Halogenase, ‘BorH’, uncultured bacteria
MDNRINRIVILGGGTAGWMTASYLAKALGDTVTITLLEAPAIGRIGVGEATVPNLQRVFFDFLGLREEEWMPECNAAFKTAVKFINWRTPGPGEAKARTIDGRPDHFYHPFGLLPEHGQVPLSHYWAYNRAAGTTDEPFDYACFAETAAMDAVRAPKWLDGRPATRYAWHFDAHLVAEFLRRHATERLNVEHVQGEMQQVLRDERGFITALRTVEGRDLEGDLFIDCSGFRGLLINKAMEEPFIDMNDQLLCNRAVATAIKHDDDAHGVEPYTSAIAMRSGWSWKIPMLGRFGTGYVYSSRFAEKDEATLDFCRMWGLDPENTPLNQVAFRVGRNRRAWVKNCVSIGLASCFLEPLESTGIYFITAAIYQLTQHFPDRTFALALSDAFNHEIEAMFDDTRDFIQAHFYVSPRTDTPFWKANKDLHLPEQMREKIAMYKAGLPINAPVTDESTYYGRFEAEFRNFWTNGSYYCIFAGLGLRPDNPLPMLRHRPEQVREAQALFAGVKDKQRELVETLPSNLEFLRSLHGK

SEQ ID NO: 53 - protein, 415 aa, Pseudomonas sp.
MKKRIGIVGAGTAGLHLGLFLRQHDVDVTVYTDRKPDEYSGLRLLNTVAHNAVTVQREVALDVNEWPSEEFGYFGHYYYVGGPQPMRFYGDLKAPSRAVDYRLYQPMLMRALEARGGKFCYDAVSAEDLEGLSEQYDLLVVCTGKYALGKVFEKQSENSPFEKPQRALCVGLFKGIKEAPIRAVTMSFSPGHGELIEIPTLSFNGMSTALVLENHIGSDLEVLAHTKYDDDPRAFLDLMLEKLGKHHPSVAERIDPAEFDLANSSLDILQGGVVPAFRDGHATLNNGKTIIGLGDIQATVDPVLGQGANMASYAAWILGEEILAHSVYDLRFSEHLERRRQDRVLCATRWTNFTLSALSALPPEFLAFLQILSQSREMADEFTDNFNYPERQWDRFSSPERIGQWCSQFAPTIAA

SEQ ID NO: 54 - protein, 542 aa, Rhodococcus erythropolis
MTTTEIPPTGVDPADSGAPQVNPAADSEANRTKNFATRPMTGDEYISSLQDGREIWLHGDRVKDVTTHPAFRNPIRMTARLYDALHTGEHVDALTVPTDTGNGGVTMPFFRTPTSSADLLKERDAIATWARMTYGWMGRSPDYKASFLGTLHANKELYAPFQDNAERWYRESQEKVLYWNHAIINPPVDRQLPPDEVGDVFMKVEKETDAGLIVSGAKVVATGSAITNYNFIAHYGLPIKKKQFALICTVPMDAPGVKLICRTSYTEHAAVMGSPFDYPLSSRMDENDTIFVFDKVLVPWENVFMYGDVDRINAFFPQSGFLPRFTFQGCTRLAVKLDFIAGLLMKALEATGAGGFRGVQTRVGEVIGWRNLFWSLTESMARDPEQWVGDSVIPKLEYGLTYRMFMIQGYPRIKEIIEQDVASGLIYLPSSSLDFKSPDVRPYLDKYVRGSDGITAVDRVKVMKALWDSIGTEFGGRHELYERNYSGNHENVKAELLFAAQNRGHASSMKGLAEQCLSEYDLDGWTVPDLIGNDDVSFFKNR

SEQ ID NO: 55 - protein, 520 aa, Klebsiella oxytoca
MKPENFRADTKRPLTGEEYLKSLQDGREIYIYGERVKDVTTHPAFRNAAASVAQLYDALHNPELQNTLCWGTDTGSGGYTHKFFRVAKSADDLRQQRDAIAEWSRLSYGWMGRTPDYKAAFGGGLGANPGFYGQFEQNARDWYTRIQETGLYFNHAIVNPPIDRHKPADEVKDVYIKLEKETDAGIIVSGAKVVATNSALTHYNMIGFGSAQVMGENPDFALMFVAPMDAEGDKLISRASYELVAGATGSPYDYPLSSRFDENDAILVMDNVLIPWENVLIYRDFDRCRRWTMEGGFARMYPLQACVRLAVKLDFITALLKRSLECTGTLEFRGVQAELGEVVAWRNMFWALSDSMCAEATPWVNGAYLPDHAALQTYRVMAPMPYAKIKNIIERSVTSGLIYLPSSARDLNNPQINDTLAKYVRGSNGMDHVERIKILKLMWDAIGSEFGGCHELYEINYSGSQDEIRLQCLRQAQSSGNMDKMMAMVDRCLSEYDQNGWTVPHLHNNTDINMLDKLLK

SEQ ID NO: 56 - protein, 517 aa, Ralstonia pickettii
MIRTGTQYLESLNDGRNVWVGNEKIDNVATHPKTRDYAQRHADFYDLHHRPDLQDVMTYIDEGGQRRAMQWFGHRDKEQLRRKRKYHETVMREMAGASFPRTPDVNNYVLTTYIDDPAPWETQSIGDDGHIKAGKIVDFIRYAREHDLNCAPQFVDPQMDRSNPDAQERSPGLRVVEKNEKGIVVNGVKAIGTGVAFADWIHIGVFFRPGIPGDQVIFAATPVNTPGVTIVCRESLVKDDKVEHPLAAQGDELDGMTVFENVFIPWSHVFHIGNPNHAKLYPQRVFDWLHYHALIRQMVRAELVAGLAVLITEHIGTNKIPAVQTRVAKLIGFHQAMLAHLIASEELGFHTPGGHYKPNILIYDFGRALYLENFSQMIYELVDLSGRSALIFASEDQWNDDKLNGWFERMNNGPVGRPHDRVKIGRVIRDLFLTDWGSRLFVFENFNGTPLQGIRMLTMQRAEFSGSGPYGKLARQVCGIDSAVTDDTEYRKTADYAKALDAARHQEEVALAGAMAI

SEQ ID NO: 57 - protein, 324 aa, Sphingobium chlorophenolicum
MTNPVSTIDMTVTQITRVAKDINSYELRPEPGVILPEFTAGAHIGVSLPNGIQRSYSLVNPQGERDRYVITVNLDRNSRGGSRYLHEQLRVGQRLSIVPPANNFALVETAPHSVLFAGGIGITPIWSMIQRLRELGSTWELHYACRGKDFVAYRQELEQAAAEAGARFHLHLDEEADGKFLDLAGPVAQAGQDSIFYCCGPEAMLQAYKAATADLPSERVRFEHFGAALTGEPADDVFTVVLARRSGQEFTVEPGMTILETLLQNGISRNYSCTQGVCGTCETKVLEGEPDHRDWVLSDEKKASNSTMLICCSLSKSPRLVLDI

SEQ ID NO: 58 - protein, 406 aa, Sphingobium baderi
MTQAIDAGERVVSLDLETHGPEALIKRAAELVPLLRENAVRCHNERRIPSENLSAMRQAGLLRMSRPRRYGGHEAMVATKTAVFGELARGCGSTAWVTTLYEDAAFLISLFPDEVQDEVFADQDTLITATLIPAGKAQPQGEGFLVNGKWPFNTGCLDATYVVEPAVVELSKGAPEVCLFLMPYSELVIEDDWSPVGLRGTGSNSVRAKDVYVRPERMLRLADVMRGIYGTKLNKGPLYRVPPIPYIVSSAGGTFPGLAQSAFELVTERLVGRPITYTLYTDRAQAPVTHLQLGEAALKIKAANHLMTEVADRLDRRALNNEALTPDEGPMIWGIIGYTSRIYAEVIESLRQMSGASALSETSAIQAVARNAQALATHAMLIPTTGIEHYGRAICGLPPNTPFLSS

SEQ ID NO: 59 - protein, 381 aa, Escherichia coli (strain K12)
MSLNMFWFLPTHGDGHYLGTEEGSRPVDHGYLQQIAQAADRLGYTGVLIPTGRSCEDAWLVAASMIPVTQRLKFLVALRPSVTSPTVAARQAATLDRLSNGRALFNLVTGSDPQELAGDGVFLDHSERYEASAEFTQVWRRLLQRETVDFNGKHIHVRGAKLLFPAIQQPYPPLYFGGSSDVAQELAAEQVDLYLTWGEPPELVKEKIEQVRAKAAAHGRKIRFGIRLHVIVRETNDEAWQAAERLISHLDDETIAKAQAAFARTDSVGQQRMAALHNGKRDNLEISPNLWAGVGLVRGGAGTALVGDGPTVAARINEYAALGIDSFVLSGYPHLEEAYRVGELLFPLLDVAIPEIPQPQPLNPQGEAVANDFIPRKVAQS

SEQ ID NO: 60 - protein, 422 aa, Acinetobacter baumannii
MENTVLNLDSDVIHACEAIFQPIRLVYTHAQTPDVSGVSMLEKIQQILPQIAKNAESAEQLRRVPDENIKLLKEIGLHRAFQPKVYGGLEMSLPDFANCIVTLAGACAGTAWAFSLLCTHSHQIAMFSKQLQDEIWLKDPDATASSSIAPFGKVEEVEGGIILNGDYGWSSGCDHAEYAIVGFNRFDADGNKIYSFGVIPRSDYEIVDNWYAQAIKSSGSKMLKLVNVFIPEYRISKAKDMMEGKSAGFGLYPDSKIFYTPYRPYFASGFSAVSLGIAERMIEAFKEKQRNRVRAYTGANVGLATPALMRIAESTHQVAAARALLEKTWEDHRIHGLNHQYPNKETLAFWRTNQAYAVKMCIEAVDRLMAAAGATSFMDNSELQRLFRDAHMTGAHAYTDYDVCAQILGRELMGMEPDPTMV

SEQ ID NO: 61 - protein, 515 aa, Burkholderia cepacia
MRTGKQYLESLNDGRVVWVGNEKIDNVATHPLTRDYAERVAQFYDLHHRPDLQDVLTFVDADGVRRSRQWQDPKDAAGLRVKRKYHETILREIAAGSYGRLPDAHNYTFTTYADDPEVWEKQSIGAEGRNLTQNIHNFLKLLREKDLNCPLNFVDPQTDRSSDAAQARSPNLRIVEKTDDGIIVNGVKAVGTGIAFGDYMHIGCLYRPGIPGEQVIFAAIPTNTPGVTVFCRESTVKNDPAEHPLASQGDELDSTTVFDNVFIPWEQVFHIGNPEHAKLYPQRIFDWVHYHILIRQVLRAELIVGLAILITEHIGTSKLPTVSARVAKLVAFHLAMQAHLIASEETGFHTKGGRYKPNPLIYDFGRAHFLQNQMSVMYELLDLAGRSSLMIPSEGQWDDSQSGQWFVKLNNGPKGNPRERVQIGRVIRDLYLTDWGGRQFMFENFNGTPLFAVFAATMTRDDMSAAGTYGKFASQVCGIEFGGAEPTAYAATADYAKALDKGLAPEPAAAESATS

SEQ ID NO: 62 - protein, 519 aa, Rhodococcus sp.
MTTSAFVDDRVGVPNDVRPMTGDEYLESLRDGREVYFRGERVDDVTTHPAFRNSARSVARMYDALHQPEQEGVLAVPTDTGNGGFTHPFFKTARSADDLVLSRDAIVAWQREVYGWLGRSPDYKASFLGTLGANADFYGPYRDNALRWYKHAQERMLYLNHAIVNPPIDRDKPADETADVCVHVVEETDAGLIVSGAKVVATGSAITNANFIAHYGLLRKKEYGLIFTVPMDSPGLKLFCRTSYEMNAAVMGTPFDYPLSSRFDENDAIMVFDRVLVPWENVFAYDTDTANGFVMKSGFLSRFMFHGCARLAVKLDFIAGCVMKGVEMTGSAGFRGVQMQIGEILNWRDMFWGLSDAMAKSPEQWVNGAVQPNLNYGLAYRTFMGVGYPRVKEIIQQVLGSGLIYLNSHASDWANPAMRPYLDQYVRGSNGVAAIDRVQLLKLLWDAVGTEFGGRHELYERNYGGDHEAVRFQTLFAYQATGQDLALKGFAEQCMSEYDVDGWTRPDLIGNDDLRIVRG

SEQ ID NO: 63 - protein, 412 aa, Variovorax paradoxus (strain EPS)
MKRIAIVGAGQSGLQLGLGLLAAGYEVTMFSNRTGEDIRRGKVMSSQCMFDTSLQIERDLGLDHWASDCPTVDGIGLAVPHPEQKGAKVIDWAARLNASAQSVDQRLKIPAWMDEFQKKGGELVFKDAGIDELEACTQSHDLTLVASGKGEISKLFERDAHKSPYDKPQRALALTYVKGMAPREPFSAVCFNLIPGVGEYFVFPALTTTGPCEIMVFEGVPGGPMDCWADVKTPEEHLARSKWILDTFTPWEAERCKDIELTDDNGILAGRFAPTVRKPVATLPSGRKVLGLADVVVLNDPITGQGSNNAAKCADTYLKSILARGDGAADAAWMQQTFDRYWFGYAQWVTQWTNMLLAPPPPHVLNLLGSAGAVPPLASAFANGFDDPRTFFPWFADAAESERYIATCAAVA

SEQ ID NO: 64 - protein, 406 aa, Rhodococcus opacus
MRKITIVGAGQAGLQLAIGLVDAGYDVTVVSNRTPEQIREGKVMSSQCMFGGALARERAVGLDLWSDECPSVEGISFTVADNGNQAFSWASRLDLPAQSVDQRVKMPAWLKEFEARGGTLVLQDAGIGDLEGFAVDSDLVILAAGKGEIAKMFERDATRSVFDRPQRALALTYVNGMVPREEHSAVAFNMIPGVGEYFAFPALTTSGPCEIMVMEGIPGGPMDCWADVTTPEEHLAKSKWVVETFVPWEAERCADITLTDDNGILAGRFPPTVRKPIGVLPSGANILGLADVVVLNDPLTGQGSNNASKCAASYLASILEHGGRPFDAEFMTDTFERYWDYAQYVASWTNALLSPPPEHVLKLLGAAATEPRIARRFANGFDDPRDFYHWFMTPEAAAQYLTDVAN

SEQ ID NO: 65 - protein, 413 aa, Acinetobacter baylyi (strain ATCC 33305 / BD413 / ADP1)
MMRRIAIVGAGQSGLQLGLSLLDTGYDVTIVTNRTADQIRQGKVMSSQCMFHTALQTERDVGLNFWEEQCPAVEGIGFTLVSPETGKPAFSWSARLERYAQSVDQRVKMPYWIEEFERRGGKLIIQDVGIDELEQLTTEYELVLLAAGKGEVVKQFVRDDERSTFDKPQRALALTYVTGMKPMSPYSRVTFNVIPGVGEYFCFPALTVTGPCEIMVFEGIPGGPMDCWQDAKTPEQHLQMSKDILNTYLPWEAERCENIEITDAGGYLAGRFPPSVRKPILTLPSGRQVFGMADALVVNDPITGQGSNNAAKCSKIYFDAILAHDTQSFTPEWMQQTFERYWSYAEKVVAWTNSLLVPPQPQMIDVLAAASQNQAIASTIANNFDDPRNFSPWWFDAEQAQHFIESKSCQKVA

SEQ ID NO: 66 - protein, 363 aa, Pseudomonas putida
MKCGFFHTPYNLPTRTARQMFDWSLKLAQVCDEAGFADFMIGEHSTLAWENIPCPEIIIGAAAPLTKNIRFAPMAHLLPYHNPATLAIQIGWLSQILEGRYFLGVAPGGHHTDAILHGFEGIGPLQEQMFESLELMEKIWAREPFMEKGKFFQAGFPGPDTMPEYDVEIADNSPWGGRESMEVAVTGLTKNSSSLKWAGERNYSPISFFGGHEVMRSHYDTWAAAMQSKGFTPERSRFRVTRDIFIADTDAEAKKRAKASGLGKSWEHYLFPIYKKFNLFPGIIADAGLDIDPSQVDMDFLAEHVWLCGSPETVKGKIERMMERSGGCGQIVVCSHDNIDNPEPYFESLQRLASEVLPKVRMG

SEQ ID NO: 67 - protein, 378 aa, Pseudomonas putida
MAMETGLIFHPYMRPGRSARQTFDWGIKSAVQADSVGIDSMMISEHASQIWENIPNPELLIAAAALQTKNIKFAPMAHLLPHQHPAKLATMIGWLSQILEGRYFLGIGAGAYPQASYMHGIRNAGQSNTATGGEETKNLNDMVRESLFIMEKIWKREPFFHEGKYWDAGYPEELEGEEGDEQHKLADFSPWGGKAPEIAVTGFSYNSPSMRLAGERNFKPVSIFSGLDALKRHWEVYSEAAIEAGHTPDRSRHAVSHTVFCADTDKEAKRLVMEGPIGYCFERYLIPIWRRFGMMDGYAKDAGIDPVDADLEFLVDNVFLVGSPDTVTEKINALFEATGGWGTLQVEAHDYYDDPAPWFQSLELISKEVAPKILLPKR

SEQ ID NO: 68 - protein, 355 aa, Vibrio harveyi
MKFGNFLLTYQPPELSQTEVMKRLVNLGKASEGCGFDTVWLLEHHFTEFGLLGNPYVAAAHLLGATETLNVGTAAIVLPTAHPVRQAEDVNLLDQMSKGRFRFGICRGLYDKDFRVFGTDMDNSRALMDCWYDLMKEGFNEGYIAADNEHIKFPKIQLNPSAYTQGGAPVYVVAESASTTEWAAERGLPMILSWIINTHEKKAQLDLYNEVATEHGYDVTKIDHCLSYITSVDHDSNRAKDICRNFLGHWYDSYVNATKIFDDSDQTKGYDFNKGQWRDFVLKGHKDTNRRIDYSYEINPVGTPEECIAIIQQDIDATGIDNICCGFEANGSEEEIIASMKLFQSDVMPYLKEKQ

SEQ ID NO: 69 - protein, 324 aa, Vibrio harveyi
MKFGLFFLNFMNSKRSSDQVIEEMLDTAHYVDQLKFDTLAVYENHFSNNGVVGAPLTVAGFLLGMTKNAKVASLNHVITTHHPVRVAEEACLLDQMSEGRFAFGFSDCEKSADMRFFNRPTDSQFQLFSECHKIINDAFTTGYCHPNNDFYSFPKISVNPHAFTEGGPAQFVNATSKEVVEWAAKLGLPLVFRWDDSNAQRKEYAGLYHEVAQAHGVDVSQVRHKLTLLVNQNVDGEAARAEARVYLEEFVRESYSNTDFEQKMGELLSENAIGTYEESTQAARVAIECCGAADLLMSFESMEDKAQQRAVIDVVNANIVKYHS

SEQ ID NO: 70 - protein, 355 aa, Photorhabdus luminescens; identical to SEQ ID NO: 68

SEQ ID NO: 71 - protein, 327 aa, Photorhabdus luminescens
MKFGLFFLNFINSTTVQEQSIVRMQEITEYVDKLNFEQILVYENHFSDNGVVGAPLTVSGFLLGLTEKIKIGSLNHIITTHHPVAIAEEACLLDQLSEGRFILGFSDCEKKDEMHFFNRPVEYQQQLFEECYEIINDALTTGYCNPDNDFYSFPKISVNPHAYTPGGPRKYVTATSHHIVEWAAKKGIPLIFKWDDSNDVRYEYAERYKAVADKYDVDLSEIDHQLMILVNYNEDSNKAKQETRAFISDYVLEMHPNENFENKLEEIIAENAVGNYTECITAAKLAIEKCGAKSVLLSFEPMNDLMSQKNVINIVDDNIKKYHMEYT

SEQ ID NO: 72 - protein, 440 aa, Geobacillus thermodenitrificans
MTKKIHINAFEMNCVGHIAHGLWRHPENQRHRYTDLNYWTELAQLLEKGKFDALFLADVVGIYDVYRQSRDTAVREAVQIPVNDPLMLISAMAYVTKHLAFAVTFSTTYEHPYGHARRMSTLDHLTKGRIAWNVVTSHLPSADKNFGIKKILEHDERYDLADEYLEVCYKLWEGSWEDNAVIRDIENNIYTDPSKVHEINHSGKYFEVPGPHLCEPSPQRTPVIYQAGMSERGREFAAKHAECVFLGGKDVETLKFFVDDIRKRAKKYGRNPDHIKMFAGICVIVGKTHDEAMEKLNSFQKYWSLEGHLAHYGGGTGYDLSKYSSNDYIGSISVGEIINNMSKLDGKWFKLSVGTPKKVADEMQYLVEEAGIDGFNLVQYVSPGTFVDFIELVVPELQKRGLYRVDYEEGTYREKLFGKGNYRLPDDHIAARYRNISSNV

SEQ ID NO: 73 - protein, 223 aa, Chelativorans multitrophicus
PAVAANYGAAIATHDNRYERAEEFLEVVHGLWNSWKFPWDEAIGPNPNPFGEVMPINHEGKYFKVAGPLNVPLPPYGPPVVVQAGGSDQGKRLASRFGEIIYAFLGSKPAGRRFVAEARAAARAQGRPEGSTLVLPSFVPLIGSTEAEVKRLVAEYEAGLDPAEQRIEALSKQLGIDLERINVDQVLQEKDFNLPKESATPIGILKSMVDVALDEKLSLRQLA

SEQ ID NO: 74 - protein, 378 aa, Streptomyces viridifaciens
MRSLDAARDTCERLHPGLIKALEELPLLEREAEGSPVLDIFRAHGGAGLLVPSAYGGHGADALDAVRVTRALGACSPSLAAAATMHNFTAAMLFALTDRVIPPTDEQKKLLARVAPEGMLLASGWAEGRTQQDILNPSVKATPVDDGFILNGSKKPCSLSRSMDILTASVILPDETGQQSLAVPLIMADSPGISVHPFWESPVLAGSQSNEVRLKDVHVPEKLIIRGTPDDPGRLDDLQTATFVWFELLITSAYVGAASALTELVMERDRGSVTDRAALGIQLESAVGLTEGVARAVRDGVFGEEAVAAALTARFAVQKTLAAISDQAIELLGGIAFIKSPELAYLSSALHPLAFHPPGRTSSSPHLVEYFSGGPLEI

SEQ ID NO: 75 - protein, 113 aa, Streptomyces coelicolor
MAEVNDPRVGFVAVVTFPVDGPATQHKLVELATGGVQEWIREVPGFLSATYHASTDGTAVVNYAQWESEQAYRVNFGADPRSAELREALSSLPGLMGPPKAVFMTPRGAILPS

SEQ ID NO: 76 - protein, 382 aa, Escherichia coli (strain K12)
MQDAAPRLTFTLRDEERLMMKIGVFVPIGNNGWLISTHAPQYMPTFELNKAIVQKAEHYHFDFALSMIKLRGFGGKTEFWDHNLESFTLMAGLAAVTSRIQIYATAATLTLPPAIVARMAATIDSISGGRFGVNLVTGWQKPEYEQMGIWPGDDYFSRRYDYLTEYVQVLRDLWGTGKSDFKGDFFTMNDCRVSPQPSVPMKVICAGQSDAGMAFSARYADFNFCFGKGVNTPTAFAPTAARMKQAAEQTGRDVGSYVLFMVIADETDDAARAKWEHYKAGADEEALSWLTEQSQKDTRSGTDTNVRQMADPTSAVNINMGTLVGSYASVARMLDEVASVPGAEGVLLTFDDFLSGIETFGERIQPLMQCRAHLPALTQEVA

SEQ ID NO: 77 - protein, 315 aa, Acinetobacter baumannii
MNQLNTAIVEKEVIDPMAFRRALGNFATGVTIMTAQTSSGERVGVTANSFNSVSLDPALVLWSIDKKSSSYRIFEEATHFGVNILSAAQIELSNRFARRSEDKFANIEFDLGVGNIPLFKNCSAAFECERYNIVEGGDHWIIIGRVVKFHDHGRSPLLYHQGAYSAVLPHPSLNMKSETAEGVFPGRLYDNMYYLLTQAVRAYQNDYQPKQLASGFRTSEARLLLVLESKTASSKCDLQREVAMPIREIEEATKILSEKGLLIDNGQHYELTEQGNACAHMLYKIAESHQEEVFAKYTVDERKLFKNMLKDLIGI

SEQ ID NO: 78 - protein, 172 aa, Bacillus subtilis subsp. natto (strain BEST195)
MLVINGTPRKHGRTRIAASYIAALYHTDLIDLSEFVLPVFNGEADQSELLKVQELKKRVTKADAIVLLSPEYHSGMSGALKNALDFLSSEQFKYKPVALLAVAGGGKGGINALNNMRTVMRGVYANVIPKQLVLDPVHIDVENATVAENIKESIKELVEELSMFAKTGNPGV

SEQ ID NO: 79 - protein, 233 aa, Escherichia coli (strain K12)
MTTLSCKVTSVEAITDTVYRVRIVPDAAFSFRAGQYLMVVMDERDKRPFSMASTPDEKGFIELHIGASEINLYAKAVMDRILKDHQIVVDIPHGEAWLRDDEERPMILIAGGTGFSYARSILLTALARNPNRDITIYWGGREEQHLYDLCELEALSLKHPGLQVVPVVEQPEAGWRGRTGTVLTAVLQDHGTLAEHDIYIAGRFEMAKIARDLFCSERNAREDRLFGDAFAFI

SEQ ID NO: 80 - protein, 170 aa, Escherichia coli
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 81 - protein, 217 aa, Escherichia coli (strain K12)
MDIISVALKRHSTKAFDASKKLTPEQAEQIKTLLQYSPSSTNSQPWHFIVASTEEGKARVAKSAAGNYVFNERKMLDASHVVVFCAKTAMDDVWLKLVVDQEDADGRFATPEAKAANDKGRKFFADMHRKDLHDDAEWMAKQVYLNVGNFLLGVAALGLDAVPIEGFDAAILDAEFGLKEKGYTSLVVVPVGHHSVEDFNATLPKSRLPQNITLTEV

SEQ ID NO: 82 - protein, 609 aa, Curvularia inaequalis
MGSVTPIPLPKIDEPEEYNTNYILFWNHVGLELNRVTHTVGGPLTGPPLSARALGMLHLAIHDAYFSICPPTDFTTFLSPDTENAAYRLPSPNGANDARQAVAGAALKMLSSLYMKPVEQPNPNPGANISDNAYAQLGLVLDRSVLEAPGGVDRESASFMFGEDVADVFFALLNDPRGASQEGYHPTPGRYKFDDEPTHPVVLIPVDPNNPNGPKMPFRQYHAPFYGKTTKRFATQSEHFLADPPGLRSNADETAEYDDAVRVAIAMGGAQALNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGVNRDLRQPYDPTAPIEDQPGIVRTRIVRHFDSAWELMFENAISRIFLGVHWRFDAAAARDILIPTTTKDVYAVDNNGATVFQNVEDIRYTTRGTREDPEGLFPIGGVPLGIEIADEIFNNGLKPTPPEIQPMPQETPVQKPVGQQPVKGMWEEEQAPVVKEAP

SEQ ID NO: 83 - protein, 371 aa, Agrocybe aegerita
MKYFPLFPTLVFAARVVAFPAYASLAGLSQQELDAIIPTLEAREPGLPPGPLENSSAKLVNDEAHPWKPLRPGDIRGPCPGLNTLASHGYLPRNGVATPVQIINAVQEGLNFDNQAAVFATYAAHLVDGNLITDLLSIGRKTRLTGPDPPPPASVGGLNEHGTFEGDASMTRGDAFFGNNHDFNETLFEQLVDYSNRFGGGKYNLTVAGELRFKRIQDSIATNPNFSFVDFRFFTAYGETTFPANLFVDGRRDDGQLDMDAARSFFQFSRMPDDFFRAPSPRSGTGVEVVIQAHPMQPGRNVGKINSYTVDPTSSDFSTPCLMYEKFVNITVKSLYPNPTVQLRKALNTNLDFFFQGVAAGCTQVFPYGRD